(19) United States
(12) Patent Application Publication
(10) Pub. No.: US 2012/0124684 A1
(43) Pub. Date: May 17, 2012

(54) COMPOSITIONS AND METHODS USING ALPHA BETA-CRYSTALLIN IN PROTECTING THE MYOCARDIUM FROM ISCHEMIA/REPERFUSION INJURY

(75) Inventors: Christopher C. Glembotski, San Diego, CA (US); Ross J. Whittaker, Encinitas, CA (US)

(73) Assignee: SAN DIEGO STATE UNIVERSITY (SDSU) FOUNDATION, San Diego, CA (US)

(21) Appl. No.: 13/132,249

(22) PCT Filed: Dec. 1, 2009
(86) PCT No.: PCT/US09/66158

§ 371 Date: Sep. 22, 2011

Related U.S. Application Data
(60) Provisional application No. 61/118,961, filed on Dec. 1, 2008.

(51) Int. Cl. A01K 67/027; C12N 5/02; C12N 5/10; A61K 38/16; C07K 14/00; C07H 21/04; C12N 5/00; C12N 15/63

(52) U.S. Cl. 800/13; 435/325; 435/375; 435/320.1; 514/21.2; 530/350; 536/23.1

(57) ABSTRACT

The invention provides a modified version of alpha B-Crystallin (αBC), and methods for delivering this modified version of alpha B-Crystallin (αBC) to tissues and cells; the invention provides a modified version of αBC in which the serine at position 59 has been changed to glutamate to mimic phosphorylation and the serine at positions 19 and 45 have been changed to alanine to prevent phosphorylation of those residues. The invention also provides compositions and methods for ameliorating a cell from ischemia/reperfusion injury or protecting a cell from ischemia/reperfusion injury.

### RELATED APPLICATIONS

This application incorporates by reference and claims the benefit of priority under 35 U.S.C. §119(e) of U.S. Provisional Application No. 61/118,961, filed Dec. 1, 2009. The aforementioned application is explicitly incorporated herein by reference in its entirety and for all purposes.

### STATEMENT AS TO RIGHTS TO INVENTIONS MADE UNDER FEDERALLY SPONSORED RESEARCH

This invention was made with government support under NIH grant no. HL085577 awarded by the National Institutes of Health (NIH), DHHS. The U.S. government has certain rights in the invention.

### TECHNICAL FIELD

This invention generally relates to cell and molecular biology, treatment or prevention of cardiac disease or injury, and medicine. In particular, the invention provides pseudo-phosphorylated alpha B-Crystallin (αBC) polypeptides, and methods for delivering this modified version of alpha B-Crystallin (αBC) to tissues and cells; and in an alternative embodiment the invention provides a modified version of αBC in which the serine at position 59 has been changed to glutamate to mimic phosphorylation and the serine at positions 19 and 45 have been changed to alanine to prevent phosphorylation of those residues.

### BACKGROUND

αBC has been shown to protect tissues and cells from a number of stresses. Phosphorylation of αBC occurs in response to various cellular stresses. It has been demonstrated that phosphorylation of the serine at position 59 is necessary and sufficient to produce the maximum protective effects of αBC.

Previous work has demonstrated that ischemia/reperfusion (I/R) results in the association of αBC with mitochondria. The presence of αBC at the mitochondria can protect against calcium-induced swelling and loss of membrane potential due to H₂O₂ treatment.

Cardiovascular disease (CVD) continues to be the leading cause of death among Americans. Of the various conditions which fall under the umbrella of cardiovascular disease, myocardial infarction (MI) is directly responsible for a significant number of the deaths attributed to CVD and is a contributing factor in mortality attributed to other forms of CVD [1]. With 1.2 million new or recurrent cases of MI reported every year, more than a third of which result in death, improving on current treatments and developing new, more effective treatments for MI are top priorities for cardiovascular researchers. One of the keys to creating new and better treatments is to understand the mechanisms employed by the heart to protect itself during stress, such as MI.

### SUMMARY

The invention provides isolated, recombinant or synthetic pseudo-phosphorylated alpha B-Crystallin (αBC) polypeptides. In alternative embodiments, the invention provides pseudo-phosphorylated alpha B-Crystallin (αBC) polypeptides comprising (a) an amino acid sequence in which the serine at position 59 has been changed to glutamate to mimic phosphorylation, and the serine at positions 19 and 45 have been changed to glycine, or an amino acid that cannot be phosphorylated in vivo, to prevent in vivo phosphorylation of those residues; or (b) the amino acid sequence of (a), wherein the serine at positions 19 and 45 have been changed to alanine to prevent phosphorylation of those residues. In alternative embodiments the invention provides an alpha B-Crystallin (αBC) amino acid sequence in which the serine at position 59, or equivalent, has been changed to glutamate to mimic phosphorylation, and the serine at positions 19 and 45 or equivalents, or the serine at position 19, or the serine at position 45, has/have been changed to glycine or to an amino acid that cannot be phosphorylated in vivo to prevent in vivo phosphorylation of those residues; or (ii)) an alpha B-Crystallin (αBC) amino acid sequence in which the serine at position 59, or equivalent, has been changed to glutamate to mimic phosphorylation, and the serine at positions 19 and 45, or the serine at position 19, or the serine at position 45, has/have been changed to alanine to prevent phosphorylation of those residues; or (iii) an alpha B-Crystallin (αBC) amino acid sequence wherein the serine at positions 19 and 45, or the serine at position 19, or the serine at position 45, has/have been changed to alanine or glycine to prevent phosphorylation of those residues.

In an alternative embodiment, the invention provides an isolated, recombinant or synthetic polypeptide comprising or consisting of

(SEQ ID NO: 1)
D IAIHHPWIRR PFFPFHAPSR LFDQFFGEHL LESDLFSTAT
SLAPFYLRPP SFLRAPEWID TGLSEMRMEK DRFSVNLDVK
HFSPEELKVK VLGDVIEVHG KHEERQDEHG FISREFHRKY
RIPADVDPLT ITSSLSSDGV LTVNGPRKQA SGPERTIPIT
REEKPAVTAA PKK;
or
(SEQ ID NO: 4)
D IAIHHPWIRR PFFPFHGPSR LFDQFFGEHL LESDLFSTAT
SLGPFYLRPP SFLRAPEWID TGLSEMRMEK DRFSVNLDVK
HFSPEELKVK VLGDVIEVHG KHEERQDEHG FISREFHRKY
RIPADVDPLT ITSSLSSDGV LTVNGPRKQA SGPERTIPIT
REEKPAVTAA PKK;
or
(SEQ ID NO: 4)
D IAIHHPWIRR PFFPFHAPSR LFDQFFGEHL LESDLFSTAT
SLGPFYLRPP SFLRAPEWID TGLSEMRMEK DRFSVNLDVK
HFSPEELKVK VLGDVIEVHG KHEERQDEHG FISREFHRKY
RIPADVDPLT ITSSLSSDGV LTVNGPRKQA SGPERTIPIT
REEKPAVTAA PKK;
or
(SEQ ID NO: 5)
D IAIHHPWIRR PFFPFHGPSR LFDQFFGEHL LESDLFSTAT
SLAPFYLRPP SFLRAPEWID TGLSEMRMEK DRFSVNLDVK
HFSPEELKVK VLGDVIEVHG KHEERQDEHG FISREFHRKY
RIPADVDPLT ITSSLSSDGV LTVNGPRKQA SGPERTIPIT
REEKPAVTAA PKK.

In alternative embodiments the alpha B-Crystallin (αBC) polypeptide is a mammalian or a human alpha B-Crystallin (αBC).

In alternative embodiments, the pseudo-phosphorylated alpha B-Crystallin (αBC) polypeptides of the invention further comprise (a) at least one protein transduction peptide domain to facilitate the delivery of the αBC protein across a cell membrane; (b) a peptide comprising a plurality of arginines, or equivalents; (c) a peptide comprising 5, 6, 7, 8, 9, or or more arginines or equivalent peptides; or (d) a peptide consisting of 5, 6, 7, 8, 9, or 10 or more arginines or equivalent peptides. The alpha B-Crystallin (αBC) polypeptide can be reversibly coupled to the peptide. The alpha B-Crystallin (αBC) polypeptide can further comprise at least one a cysteine residue. The alpha B-Crystallin (αBC) polypeptide can be coupled to the peptide via a disulfide bond between cysteine residues.

In alternative embodiments, the invention provides compositions comprising the alpha B-Crystallin (αBC) polypeptide of the invention. The composition of the invention can be formulated as a liquid, gel, solid or powder, or an enteral or parenteral formulation, or an implant.

In alternative embodiments, the invention provides pharmaceutical compositions comprising the alpha B-Crystallin (αBC) polypeptide of the invention, which can further comprise a pharmaceutically acceptable excipient.

In alternative embodiments, the invention provides uses of the alpha B-Crystallin (αBC) polypeptide of the invention to make a pharmaceutical composition to protect cells from ischemia/reperfusion injury. In one aspect, the alpha B-Crystallin (αBC) polypeptide is used make a pharmaceutical composition to protect heart or muscle cells from ischemia/reperfusion (I/R) injury.

In alternative embodiments, the invention provides methods for ameliorating a cell from ischemia/reperfusion injury or protecting a cell from ischemia/reperfusion injury, comprising: (a) providing the pharmaceutical composition of the invention, or the alpha B-Crystallin (αBC) polypeptide of the invention; and (b) administering (contacting) the pharmaceutical composition or alpha B-Crystallin (αBC) polypeptide of (a) to a cell, tissue, organ or individual in need thereof. The cell, tissue, organ can be a heart, heart tissue or heart muscle or cardiac muscle.

In alternative embodiments, the invention provides isolated, recombinant or synthetic nucleic acids encoding the alpha B-Crystallin (αBC) polypeptide of the invention.

In alternative embodiments, the invention provides expression vehicles and vectors comprising the nucleic acid of the invention.

In alternative embodiments, the invention provides host cells comprising: the alpha B-Crystallin (αBC) polypeptide of the invention, or the nucleic acid of the invention, or the expression vehicle or vector of the invention.

In alternative embodiments, the invention provides non-human transgenic animals comprising the nucleic acid of the invention, or the expression vehicle or vector of the invention.

The details of one or more embodiments of the invention are set forth in the accompanying drawings and the description below. Other features, objects, and advantages of the invention will be apparent from the description and drawings, and from the claims.

All publications, patents, patent applications, GenBank sequences and ATCC deposits, cited herein are hereby expressly incorporated by reference for all purposes.

### DESCRIPTION OF DRAWINGS

FIG. 1 illustrates a schematic of the events leading to I/R-induced cell death, as discussed in Example 1, below.

FIG. 2 illustrates a schematic of the major antioxidant systems of the heart, as discussed in Example 1, below.

FIG. 3 illustrates a schematic of the MAPK signaling pathways in I/R, as discussed in Example 1, below.

FIG. 4 illustrates a schematic of the αBC protein structure and phosphorylation by MAPK pathways, as discussed in Example 1, below.

FIG. 5 illustrates a schematic of an alternative embodiment—an alternative mechanism of action—of this invention (noting the invention is not limited by any particular mechanism of action), as discussed in Example 1, below.

FIG. 6A illustrates a schematic of the I/R protocol used in this study and FIG. 6B illustrates an electrophoretic analysis of the subcellular fractionations for organelle contamination, as discussed in Example 1, below.

FIG. 7A illustrates an electrophoretic analysis of the effect of ischemia or ischemia/reperfusion on αBC, phospho-αBC-559, p38 and Phospho-p38 in whole heart homogenates, and FIGS. 7B and 7C schematically summarize the results of that analysis, as discussed in Example 1, below.

FIG. 8 illustrates an electrophoretic analysis of the effect of ischemia and ischemia/reperfusion on αBC in cytosolic fractions, as discussed in Example 1, below.

FIG. 9A illustrates an electrophoretic analysis of the effect of ischemia and ischemia/reperfusion on αBC, phospho-αBC-559, p38, and phospho-p38 in mitochondrial fractions, and FIGS. 9B and 9C schematically summarize the results of that analysis, as discussed in Example 1, below.

FIGS. 10A and 10B illustrate an electrophoretic analysis of the effect of Trypsin on Mitochondrial αBC, and FIG. 10C schematically summarizes the results of that analysis, as discussed in Example 1, below.

FIGS. 11A and 11C illustrate an electrophoretic analysis of the effect of wild type αBC-AdV and αBC-AAE-AdV on H₂O₂-induced Cytochrome C release, and FIG. 11B schematically summarizes the results of that analysis, as discussed in Example 1, below.

FIGS. 12A and 12B illustrate an electrophoretic analysis of the co-immunoprecipitation of αBC and VDAC from neonatal rat cardiomyocyte mitochondria (NRVCM) and mouse heart mitochondria, as discussed in Example 1, below.

FIG. 13A illustrates an electrophoretic analysis of a proteomics analysis of interactions between αBC and components of the mitochondria, and FIG. 13B schematically summarizes the results of MudPIT (Multidimensional Protein Identification) analysis for αBC immunoprecipitations, as discussed in Example 1, below.

FIG. 14 illustrates a summary of findings regarding αBC translocation to the mitochondria, as discussed in Example 1, below.

FIGS. 15A, 15B and 15C schematically summarize and illustrate data of the measurement of total glutathione, oxidized glutathione, and reduced glutathione to oxidized glutathione ratios in hearts from αBC/HSPB2 knockout mice, as discussed in Example 1, below.

FIGS. 16A and 16B schematically summarize and illustrate data of the measurement of G6PD, GR activity and protein levels in αBC/HSPB2 knockout mouse hearts, and FIG. 16C illustrates an electrophoretic analysis of that study, as discussed in Example 1, below.

FIGS. 17A and 17B illustrate an electrophoretic analysis of the comparison of protein expression in αBC/HSPB2 KO hearts and HeLa cells, as discussed in Example 1, below.

FIGS. 18A, 18B and 18C schematically summarize and illustrate data of the measurement of total glutathione, oxidized glutathione and reduced glutathione to oxidized glutathione ratios in HeLa cells transfected with αBC plasmid, as discussed in Example 1, below.

FIGS. 19A, 159 and 19C schematically summarize and illustrate data of the measurement of total glutathione, oxidized glutathione and reduced glutathione to oxidized glutathione ratios in HeLa cells transfected with αBC plasmid following 90 min treatment with 200 μM H2O2, as discussed in Example 1, below.

FIGS. 20A and 20B schematically summarize and illustrate data of the measurement of G6PD, GR activity and protein levels in αBC/HSPB2 knockout mouse hearts, and FIG. 20C illustrates an electrophoretic analysis of that study, as discussed in Example 1, below.

FIG. 21 schematically summarizes and illustrates data of the measurement of protection against hydrogen peroxide-induced activation of apoptosis by αBC expression, as discussed in Example 1, below.

FIG. 22A illustrates an electrophoretic analysis of the co-immunoprecipitation of αBC and GR and FIG. 22B schematically summarizes and illustrates data of the measurement of enhancement of GR activity in vitro by purified αBC, as discussed in Example 1, below.

FIG. 23 schematically illustrates and summarizes findings regarding the effects of αBC expression on gluthathione recycling, as discussed in Example 1, below.

FIG. 24A schematically illustrates exemplary cloning constructs designs for expression of compositions (polypeptides) of this invention; FIG. 24B illustrates an electrophoretic analysis of the expression of these constructs in bacteria; and FIG. 24C illustrates an electrophoretic analysis of the purification of expressed tat-αBC proteins, as discussed in Example 1, below.

FIG. 25A schematically illustrates and summarizes findings regarding the uptake of tat-αBC protein by NRVCM's; and FIG. 25B schematically illustrates a confocal examination of Tat-αBC protein uptake, as discussed in Example 1, below.

FIG. 26A schematically illustrates and summarizes findings regarding heat shock-induced translocation of αBC and FIG. 26B schematically illustrates data showing an ADH aggregation assay, and the inappropriate partitioning and instability of the tat-αBC proteins, as discussed in Example 1, below.

FIG. 27A illustrates electrophoretic analysis of the in vitro linkage of the AAC and the R9-C delivery platform; and FIG. 27B illustrates electrophoretic analysis of delivery of the AAE-R9c into HeLa cells, as discussed in Example 1, below.

FIG. 28A schematically illustrates the amino acid sequence of domains in the exemplary protein of the invention comprising the AAE protein, including the αB crystalline sequence, the location of AAE modifications, a cysteine added to permit linkage to R9c, the nine amino acids that remain following enterokinase cleavage, and the sequence removed following enterokinase cleavage; FIG. 28B shows the R9 peptide, and FIG. 28C schematically illustrates this exemplary molecule of the invention ready for application (administration), as discussed in Example 1, below.

Like reference symbols in the various drawings indicate like elements.

### DETAILED DESCRIPTION

The invention provides a modified version of the protective protein, alpha B-Crystallin (αBC), and methods for delivering this modified version of alpha B-Crystallin (αBC) to tissues and cells. In one embodiment, the invention provides a modified version of αBC in which the serine at position 59 has been changed to glutamate to mimic phosphorylation and the serine at positions 19 and 45 have been changed to alanine to prevent phosphorylation of those residues. This embodiment is referred to as AAE. Delivery of this pseudo-phosphorylated form of αBC is accomplished by reversibly coupling the protein to a peptide consisting of nine arginines and a cysteine (R9). In one embodiment, the R9 is coupled to the exemplary AAE via a disulfide bond between cysteine residues.

As the amino acid sequence for native αBC does not contain a cysteine, in one embodiment the exemplary AAE is modified further in order to facilitate the linkage. In one embodiment, an exemplary AAE is inserted into the bacterial expression vector PrSet A, downstream of a 6× histidine tag that allows for purification of the protein. In this embodiment, AAE is also located downstream of an enterokinase cleavage site, which allows for removal of the 6× histidine tag and related sequence following purification. Following enterokinase cleavage, nine amino acids that are not native to αBC remain on the N-terminus of AAE. In one embodiment, one of the amino acids within these extra 9 amino acids is added to AAE is changed to a cysteine to allow formation of the disulfide bond between the R9 peptide and AAE.

In one embodiment, the disulfide bond is created by incubating the synthetic R9 with the recombinant AAE in 50 mM Tris pH 7.4 for one hour at room temperature. The combined product is an embodiment of the invention, designated “R9-AAE”. When it is delivered to either cells or tissues, it enters the cells by virtue of the R9 addition, and once inside cells, the disulfide bond is broken due to the reducing environment found in the cell. AAE is therefore liberated from the R9 peptide, which is required for AAE to exert its protective effects in cells and tissues.

a. Non-technical Description: The invention provides compositions and methods for delivering a protective protein directly to tissues and cells. In one embodiment, a protein alpha B-Crystallin is modified to enhance its protective capabilities and then combined with a synthetic peptide that facilitates its delivery to tissues, where it has maximal protective effects.

### Kits and Libraries

The invention provides kits comprising compositions of this invention and methods of the invention, including alpha B-Crystallin compositions and/or alpha B-Crystallin-encoding nucleic acids of the invention, including vectors, recombinant viruses and the like, transfecting agents, transducing agents, cardiac or vascular cells and/or cell lines, instructions (regarding the methods of the invention), or any combination thereof. As such, kits, cells, vectors and the like are provided herein.

The invention will be further described with reference to the following examples; however, it is to be understood that the invention is not limited to such examples.

### Example 1

### Demonstrating the Therapeutic Efficacy of the Compositions and Methods of this Invention

This example demonstrates that the therapeutic efficacy of the compositions and methods of the invention:

Expression of αBC in the heart has been implicated in providing protection from acute stresses, such as ischemia and reperfusion, by binding to specific target proteins. This invention explores two new mechanisms by which αBC can protect the heart from I/R. First, the role of αBC in maintaining mitochondrial integrity and function during I/R is examined. This invention provides and describes an examination of the kinetics of αBC translocation to the mitochondria as well as phosphorylation of αBC, an event that is required for αBC to provide maximal protection against stress. This study shows that, in response to ischemia, αBC rapidly leaves the cytosol and translocates to other cellular locations, including the mitochondria, where it reaches maximal accumulation following 20 minutes of ischemia. Furthermore there is an accumulation of phospho-αBC-S59, the most protective form of αBC, at the mitochondria.

This study goes further to demonstrate that the presence of a phospho-αBC-S59 mimic, αBC-AAE, at the mitochondria provides enhanced protection against H₂O₂-induced cytochrome c release. Moreover, this study demonstrates that αBC physically interacts with VDAC, a possible mechanism by which αBC prevents cytochrome c release. The study also finds that αBC may enter the mitochondria and interact with key metabolic proteins.

The second study explores a role for αBC in regulating the cellular redox environment. Hearts from mice harboring deletions of both αBC and HSPB2 were found to have significantly lower levels of glutathione compared to controls. Consistent with this finding, glutathione reductase (GR) activity was significantly impaired in the knockout mice. HeLa cells were used as a model to further examine a role for αBC in glutathione recycling since, like the knockout mice, they lack both αBC and HSPB2. Expression of αBC in HeLa cells was found to enhance glutathione levels during H₂O₂ treatment and was found to increase GR activity. This resulted in reduced caspase 3 activity following H₂O₂ treatment in HeLa cells transfected with αBC. Moreover, αBC was shown to directly interact with GR and enhance its activity.

The third study of this invention examines the use of protein transduction domains to facilitate the delivery of αBC proteins across cell membranes. This study introduces a novel system that could be used as a research or a therapeutic platform for delivering proteins, nucleic acids or small molecules.

### Chapter One

### A. Ischemia/Reperfusion Injury of the Myocardium

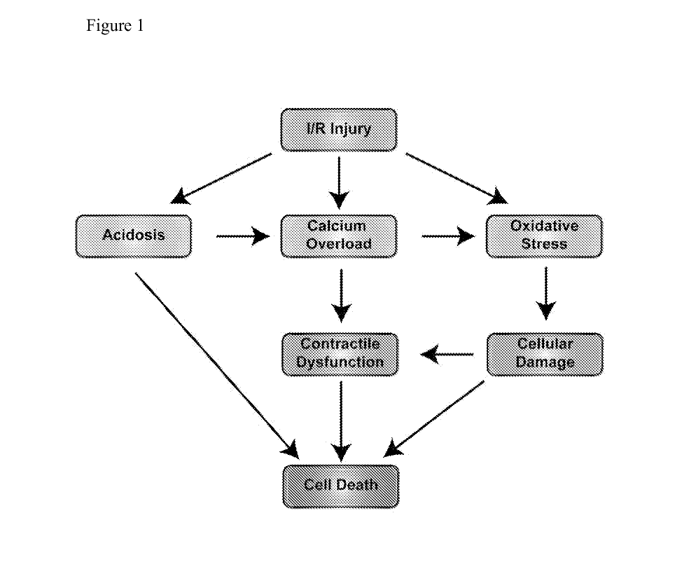

MI occurs when there is a blockage of the coronary arteries. During an MI, tissue immediately downstream of the blockage is deprived of oxygen and nutrients and there is a decrease in the clearance of CO₂. In this state the tissue is described as being ischemic. The myocytes within this ischemic region of the heart face an intertwined series of events that challenge their ability to survive and contribute to the contractile action of the heart, as illustrated in FIG. 1—a schematic of the events leading to I/R-induced cell death FR results in a complex series of events that can lead to cellular damage, and ultimately cell death, if the myocyte is unable to coordinate an appropriate pro-survival response.

The lack of oxygen and nutrients due to MI results in the collapse of oxidative phosphorylation (OxPhos)[2]. In response the myocyte can, for short periods of time, rely on anaerobic glycolysis to provide small quantities of ATP. However, anaerobic glycolysis does not generate enough ATP to maintain normal cardiomyocyte function. As a result, ATP is rapidly depleted following the onset of ischemia[3]. A consequence of resorting to glycolysis is the accumulation of lactic acid. The lack of blood flow to the area also results in an accumulation of CO₂ from the waning 0× Phos activity in the cell. This contributes to a decrease in the pH of the myocyte, a condition known as acidosis[4]. A more acidic environment by itself may prime the cell for apoptosis through activation of proapoptotic Bcl family members, such as Bnip3, and activation of caspase 3 via cytochrome c release from the mitochondria[5]. However, acidosis is also a contributing factor to the misregulation and accumulation of calcium within the myocyte during ischemia

The calcium concentration of the intracellular environment, and between the different organelles within the myocyte, is controlled by a number of channels, pumps, and transporters that can all be affected by ischemia. As protons accumulate during ischemia (acidosis), the H⁺/Na⁺ antiporter drives an accumulation of intracellular Na⁺, this in turn causes the Na⁺/Ca⁺ antiporter to pump calcium into the cell resulting in increases in cytosolic calcium[6, 7]. The depletion of ATP following the collapse of 0× Phos further contributes to the increase in intracellular calcium concentration. The sarcolemmal calcium ATPase pumps calcium across the plasma membrane and out of the cell. The sarco/endoplasmic reticulum ATPases (SERCA's) regulate calcium within the sarcoplasmic and endoplasmic reticulum (SR and ER, respectively), and their activities are vital to the cycle of calcium induced calcium release that regulates contraction. The failure of the cell to be able to provide enough ATP during ischemia to power these pumps exacerbates the accumulation of calcium during ischemia[3]. The result of this unregulated increase in intracellular calcium is commonly referred to as calcium overload.

Calcium overload is a significant contributing factor to myocyte death during an MI[8]. Calcium overload has direct effects on mitochondria where it can cause swelling of the organelle and [9] mitochondrial permeability transition pore opening (MPTP). This can result in release of cytochrome c and activation of the apoptosome resulting in cleavage and activation of caspases. Increased cytosolic calcium can also result in the activation of calpains, a group of cysteine proteases[10]. The activation of calpains can cleave and activate proapoptotic proteins such as Bax and Bid[11]. Calpains have also been found to directly target structural and cell cycle proteins and may work in conjunction with effector caspases during apoptosis.

Regulation of calcium is important to all cells, but the regulation of calcium in myocytes is even more important, since myocytes rely on strictly controlled calcium levels to regulate contraction, as well as intracellular signaling pathways[12]. Contraction is a calcium regulated event that relies on the binding and cleavage of ATP by the myosin head to provide the energy necessary to complete a contraction event. During ischemia, the combination of low levels of ATP and high levels of calcium results in an unregulated, sustained contraction of the sarcomeres referred to as contracture[8]. During contracture the intense, sustained forces result in damage and unfolding of the sarcomeric proteins. Stretch sensors associated with the sarcomeres, such as muscle LIM protein, can activate apoptosis in response to this type of damage. For myocytes that do not enter apoptosis, this damage can hinder their ability to create contractile force, which can severely reduce the heart's ability to pump blood.

Hopefully, an MI patient survives the initial blockage of the arteries and has blood flow restored to the ischemic tissue through angioplasty, bypass, or thrombolytic drugs[13]. However, they are now faced with the so-called “Oxygen Paradox” [14]. The return of blood flow brings much needed oxygen and nutrients to the previously ischemic tissue, but at the same time results in a burst of damaging reactive oxygen species (ROS) during the first few minutes of reperfusion.

This burst of reactive oxygen species can be partially attributed to calcium overload of the mitochondria during ischemia. The elevated calcium levels experienced during ischemia inhibit dephosphorylation and subsequent inactivation of NADH dehydrogenase in the mitochondria (Complex I) while also inhibiting ATP inhibition of cytochrome c oxidase (Complex IV)[15]. As a result when 0× Phos is restarted following reperfusion the respiratory chain is primed to operate at a very high (Complex I) but not very efficient (Complex IV) level, the result is a rapid rise in mitochondrial membrane potential that leads to increased ROS production.

Superoxide anion is the primary ROS species generated during reperfusion, and is quickly converted to H₂O₂ by the activity of superoxide dismutases (SOD's) [16]. Hydrogen peroxide can in turn react with ferric, ion via the Fenton reaction, to generate hydroxyl radicals, strong oxidants with the potential to cause significant cellular damage[3]. Reactive oxygen species have the potential to damage every major macromolecule in the cell [17]. They create DNA adducts and strand breaks which hinder replication and transcription and may lead to mutation. These oxidants result in lipid peroxidation, which can alter the fluidity and function of membranes. They also attack peptide bonds and reactive amino acid side chains of proteins, altering or even destroying protein structure and function. These damaging effects can lead to cellular dysfunction and, ultimately, to cell death through either necrotic or apoptotic pathways [9, 18].

### B. The Cardiomyocyte Response to I/R Injury

Cardiomyocytes subjected to I/R injury will either die by necrosis or apoptosis or they will survive the injury and recover. In order to survive I/R the myocytes must mount an extremely complex stress response in an attempt to restore homeostasis.

1. Antioxidants

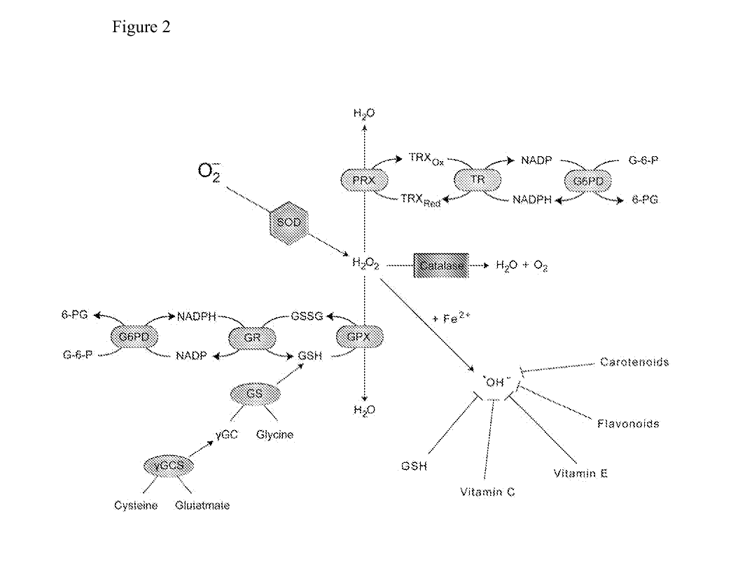

The heart requires large amounts of ATP to continuously circulate blood throughout the body. As such, cardiomyocytes are packed with mitochondria; but, being a cell type with such a high metabolic rate means cardiomyocytes must constantly manage ROS. Even when the myocyte is not, stressed up to 2-5% of oxygen consumed by OxPhos results in ROS generation[15, 19]. However, when the cells are stressed during I/R, the rate of ROS release can increase 7.5 fold[20]. In order to prevent ROS-induced cell death under both basal and stressed conditions it is imperative that the cardiomyocyte possess a robust network of antioxidants [21] (FIG. 2).

a. Superoxide Dismutase

Superoxide anion produced in the mitochondria is the primary source of ROS generated from the mitochondria under both basal metabolic conditions and during stresses such as I/R[15]. When compared to other ROS species, superoxide anion by itself is relatively slow to react with other cellular components. However, it still needs to be metabolized rapidly in order to prevent damage and subsequent generation of other ROS species. This is accomplished by a group of enzymes that convert the superoxide anion to hydrogen peroxide as the first step in detoxifying ROS. [17] There are two classes of superoxide dismutases (SOD) that are defined by their associated metals and are segregated into different cellular compartments. Copper/Zinc superoxide dismutase (SOD1) exists in the cytoplasm, while manganese superoxide dismutase (SOD2) is found exclusively in the mitochondria. Overexpression by transgenesis or viral transfer of either form of SOD has been found to provide protection against I/R injury in the heart [22, 23].

FIG. 2—The Major Antioxidant Systems of the Heart

The heart detoxifies ROS by utilizing a robust network of antioxidants. Glutathione is considered the first line of defense from ROS-related damage and exists as a redox couple between its reduced and oxidized forms. The redox potential of the glutathione couple is primarily maintained by the glutathione recycling enzymes G6PD and GR. Thioredoxin is utilized in a similar manner to glutathione and is also recycled in a similar manner by thioredoxin reductase. Catalase can directly convert H₂O₂ to H₂O and O₂ by way of a very elegant detoxifying reaction. Catalase has a very high specific activity, however, it is found in low quantities in the heart. The ROS scavengers, such as vitamins C and E and the carotenoids and flavonoids are the last line of defense against ROS-induced damage.

b. Glutathione and Associated Antioxidant Enzymes

Glutathione is considered the first line of defense against ROS damage[24]. Alterations in glutathione levels cause significant changes in the hearts ability to respond to I/R. The glutathione synthesis inhibitor, buthionine sulphoxamine, has been used to deplete the glutathione pool of hearts prior to I/R challenge, and results in reduced functional recovery when compared to untreated hearts[25, 26]. Depletion of glutathione has also been shown to trigger loss of mitochondrial membrane potential and mitochondrial membrane potential fluctuations, as well as ROS release from mitochondria in guinea pig hearts even without I/R challenge[19]. In contrast, oral supplementation of GSH in exercise-trained rats has been found to protect hearts against I/R[27].

Glutathione is synthesized from cysteine, glutamate, and glycine in a two step process by the enzymes γ-glutamylcysteine-synthetase (rate limiting) and glutathione synthetase[28]. Glutathione is ubiquitous within tissues and is also found circulating in the blood and in intercellular spaces. In the heart glutathione exists in concentrations of up to 8 mM [29]. This places the heart toward the upper end of tissue glutathione concentrations which are generally found at a concentration of 1-10 mM [30].

Glutathione exists as a redox couple between the reduced form, GSH, and its oxidized form, GSSG. Under basal conditions, 95% of cellular glutathione exists in the reduced form, resulting in a high ratio of GSH:GSSG [31]. During stress, such as I/R, when increased amounts of ROS are being generated, the ratio of GSH:GSSG falls as a result of glutathione being utilized to detoxify oxidants, as the cell tries to prevent cellular damage from occurring (Figure-Glutathione Cycle)[24, 32]. Glutathione is primarily utilized to detoxify the hydrogen peroxide that results from the dismutation of the superoxide radical that is generated by the mitochondria during I/R. The reaction is catalyzed by glutathione peroxidase or peroxiredoxin and results in hydrogen peroxide being reduced to water and GSSG [28, 29].

When glutathione is oxidized, either through direct interaction with an oxidizer or through its participation in an enzyme regulated redox reaction, GSSG is formed. At high concentrations, GSSG becomes toxic, as it can form mixed protein disulfide through disulfide exchange. These mixed protein disulfides can then react with themselves to form protein-protein thiols which results in loss of protein function and a decrease in protein stability that can lead to aggregation [33].

The cell has two routes through which it can eliminate GSSG. The first is by exporting GSSG out of the cell by way of the multi drug resistance associated protein (MRP1) [34]. However, this results in a reduction of the cell's glutathione pool and a diminished capacity to detoxify ROS[25, 26]. The second route is by recycling GSSG back to the reduced form, GSH [35]. In order to recycle GSSG the cell relies on NADPH produced by glucose-6-phosphate dehydrogenase (G6PD). NADPH is utilized by glutathione reductase (GR) to catalyze the reduction of GSSG resulting in the reduced form of glutathione, GSH, and NADP⁺.

Alterations in the levels and activity of the glutathione associated antioxidant enzymes have been shown to alter the ability of cells to respond to oxidative stress. Inhibiting the activity of GR in cardiomyocytes results in a 50% decrease in intracellular glutathione [28]. Similarly, mice harboring a mutation in the 5′ untranslated region of G6PD, resulting in an 80% reduction in G6PD activity, demonstrate significantly lower glutathione levels compared to wildtype controls[36]. Consistent with this reduction in glutathione levels, hearts from these G6PD deficient mice are more susceptible to I/R injury. On the other hand, overexpression of G6PD has been shown to be effective in preventing glutathione depletion during oxidative stress [37]. Mice with deficiencies in SOD and GPX exhibited an increased sensitivity to ROS when compared to control animals, which results in premature death when animals are challenged with paraquat[38]. In fact, SOD knockout has been demonstrated to be lethal, with animals dying shortly after birth [39]. In contrast, transgenic mice that over-express either GPX or SOD have been found to be protected from I/R injury [40, 41]. Furthermore, administration of ebselen, a glutathione peroxidase mimicking compound, reduced infarct size in a canine model of I/R [42]. Injection of adenoviral constructs expressing SOD, following coronary artery occlusion in rat hearts, resulted in a 50% reduction in infarct size [23].

c. Other Enzymatic Antioxidants

There also exists a set of enzyme antioxidants that do not rely on glutathione to detoxify ROS; catalase is one such enzyme. The catalase monomer is roughly 500 amino acids and contains a heme group. The assembled catalase enzyme is a tetramer with an Fe³⁺ center that detoxifies 2 molecules of H₂O₂ at a time to produce 2 molecules of H₂O and one molecule of O_{2 [}43]. The reaction involves two steps, whereby the first molecule of H₂O₂ enters the active site and one oxygen is transferred to the iron center and H₂O is released. When a second molecule of H₂O₂ enters the active site it reacts with the oxygen in the Fe³⁺ core resulting in the release of H₂O and O₂. Catalase not only performs a very elegant reaction to detoxify hydrogen peroxide, but also performs this reaction at an extremely high rate [44]. In fact, catalase may have the highest catalytic turnover of any enzyme and is believed to operate near the limit of enzymatic efficiency. However, the contribution of catalase in detoxifying H₂O₂, has been shown to be much lower than that of glutathione peroxidase [29]. However, hearts from transgenic mice that over-express 60-fold more catalase than wild type animals, were found to be protected from I/R, due to an increased ability to detoxify ROS [45].

Thioredoxin is an encoded small protein that acts as both an antioxidant and an important signaling molecule [46]. Thioredoxin is a ubiquitously expressed, 12 kilodalton protein with a redox reactive core containing two cysteine residues, that when oxidized, form a disulfide bridge. Oxidized thioredoxin is recycled back to the reduced form by thioredoxin reductase, utilizing NADPH as an electron donor. In vivo models have demonstrated that thioredoxin can protect the heart against I/R injury, even by simply administrating an intraperitoneal injection of thioredoxin prior to coronary artery ligature[47-51]. Thioredoxin also acts a signaling molecule and may be used as a sensor of redox status. In its reduced form, it binds to and inhibits apoptosis regulating kinase 1 (ASK1) [52]. Upon oxidation of thioredoxin ASK1 is released, resulting in the activation of downstream kinases such as p38 and INK. This regulation of signaling molecules may be an important contributing factor in myocardial hypertrophy and the progression of heart failure [53].

d. Non-Enzymatic Antioxidants

Non-enzymatic antioxidants are generally ROS scavengers derived from nutritional sources and include vitamin E and vitamin C, flavonoids and carotenoids [16]. Vitamin E is the major lipid soluble antioxidant, and is believed to play a role in protecting membranes from ROS induced damage. Vitamin C is a small soluble antioxidant that acts both as a ROS scavenger and a reducing agent for oxidized vitamin E. Studies in mice and rats have demonstrated that manipulating levels of vitamin E and/or vitamin C through supplementation can result in a significant reduction in I/R associated damage in both in vivo and ex vivo models [54-56]. However, human studies using large groups of participants have found mixed results regarding the protective effects of antioxidant supplementation [57, 58].

2. Signaling Pathways Activated by I/R Injury

Following an I/R injury, myocytes will progress through three temporally separate, but related phases, which have distinct signal transduction profiles. The first phase occurs minutes to hours following the onset of I/R and is concerned with survival of the myocyte. The second phase, occurring hours to days later, concerns recovery of the myocyte from injury. The third phase, which takes place over days, months and even years following I/R, concerns the myocytes role in the remodeling of the myocardium in response to injury.

The scope and coordination of the myocyte response to I/R will probably not be truly understood until the promise of systems biology is fulfilled. Nonetheless, significant progress has been made in understanding the pathways utilized by these specialized cells to survive I/R and help maintain organ function. The activation or deactivation of signaling pathways in response to I/R results in up and down-regulation of gene expression, reorganization of signaling complexes and changes in their activity, alterations in contraction, activation of both cell death and survival signaling, metabolic changes, and changes in communication with surrounding cells, just to name a few. We probably only understand a few of the possible outcomes for activating or deactivating any given pathway and even less about the extent of cross talk between the different signaling pathways.

For the purposes of this invention, I will touch briefly on a couple of the major signaling pathways involved in survival signaling during the acute response to I/R. I will then elaborate further on the mitogen activated protein kinase family as this group of proteins is directly related to the subject of this invention.

a. PI3K/AKT Signaling Pathway

The PI3K/AKT signaling pathway is one of the most well studied signal transduction pathways in the heart, and plays a role in regulating growth, survival, and metabolism. I/R activates PI3K which results an increase of phosphoinositol 3,4,5 triphosphosphate; this, in turn, results in the recruitment of AKT to the plasma membrane [59]. At the plasma membrane AKT is activated via dual phosphorylation by PDK1 on threonine 308, followed by a second phosphorylation on serine 473, by a currently unknown kinase. Once activated, AKT translocates away from the membrane into the cytosol and nucleus, where it phosphorylates target proteins.

Activation of AKT through extracellular signaling molecules such as IGF-1, or drugs such as vanadate and isoflourane is associated with protection of myocytes against IR damage[60-62] [63, 64]. Constitutive activation of AKT in transgenic models through the addition of a myristoyl moiety, which targets AKT to the plasma membrane, has been shown to reduce infarct size and inhibit apoptosis in hearts exposed to I/R[65].

AKT acts on a number of targets both in the cytosol and the nucleus to provide protection against I/R [59]. AKT inhibits apoptosis through phosphorylation of the proapoptotic Bcl-2 family member, BAD, resulting in its sequestration in the cytosol. Phosphorylation of pro-caspase 9 by AKT blocks its activation and prevents apoptosis. AKT also results in the activation of pro-survival transcription factor nuclear factor kappa B (NFκB) and stabilization of the anti-apoptotic transcription factor β-catenin AKT is also involved in cross talk with a number of other signaling pathways that are activated during I/R.

b. Protein Kinase C

There are four isoforms of protein kinase C (PKC) expressed in the heart (α, β, δ, ε) [66]. PKC activation occurs in a two step process requiring phosphorylation within the activation loop and subsequent interaction with the second messengers Ca²⁺ and diacylglycerol. Upon activation the different isozymes are targeted to unique subcellular locations by interactions with RACK (receptors for activated C-kinase) proteins [67]. Synthetic peptides that mimic the interaction sites of the RACK proteins can be used to activate PKC. Conversely, synthetic peptides that mimic the RACK interaction domain of PKC can be used to block PKC activation. These strategies have been used extensively to elucidate the different roles of PKCδ and PKCε.

Activation of PKCδ and PKCε seem to play opposing roles during I/R [68, 69]. Following activation, PKCδ translocates to the mitochondria and increases apoptosis. Inhibition of PKCδ results in significant reduction in cellular damage following I/R, and drastically reduces infarct size [70, 71]. Conversely, activation of PKCε by perfusion of synthetic peptides that mimic the PKC binding site of the RACK proteins results in a significant decrease in infarct size and cellular damage due to I/R [72-75].

c. Mitogen Activated Protein Kinases

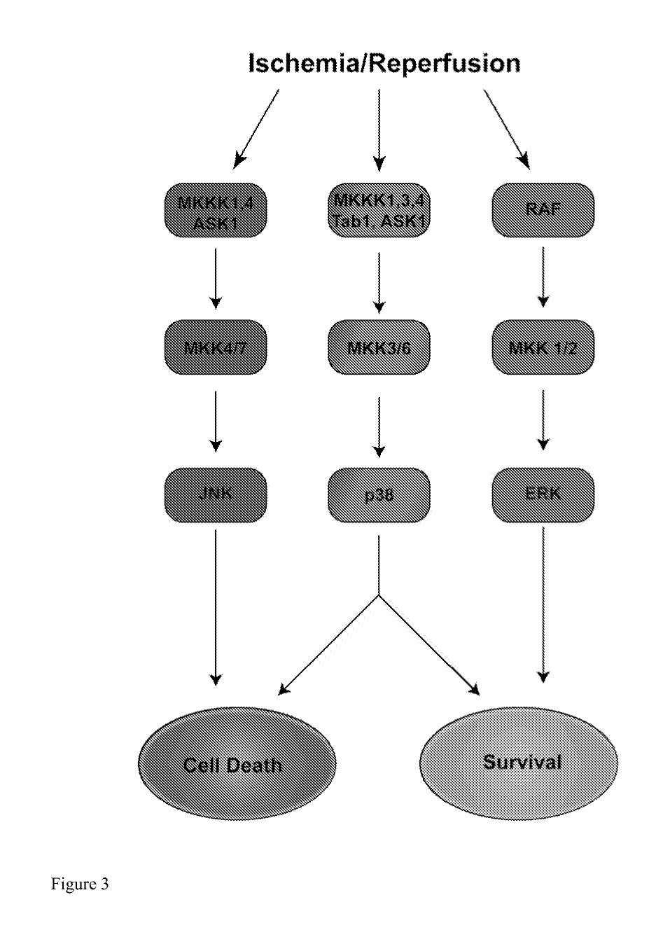

The mitogen activated protein kinases (MAPK's) are a group of protein kinases that are activated during stress. The three MAPK signaling pathways are named for the end effector kinase which they activate. The MAPK signaling pathways have been implicated in a number of different cellular processes including growth, development, inflammation and survival [76-78]. As such their activation during I/R and its consequences for the heart have been, and continue to be, an area of intense interest among cardiovascular researchers (FIG. 3).

1. ERK

Extracellular signal regulated kinase (ERK) is classically activated by extracellular mitogens such as growth factors and hormones [77]. ERK is activated via the ras-raf-MKK1/2 signaling pathway resulting in dual phosphorylation of threonine 202 and tyrosine 204 of ERK1 or threonine 185 and tyrosine 187 of ERK2. ERK has not been found to be activated by ischemia alone, but it has been found to be activated by I/R. The upstream activator of ERK, MKK2, was also found to be activated by I/R. Further studies by Li et al. demonstrated that nitric oxide (NO) produced during reperfusion may be responsible for ERK activation [79]. ERK has also been shown to be activated in an oxidative stress dependent manner by the PKC-mediated activation of RAF [80].

Multiple studies have demonstrated that ERK plays a protective role in the heart during I/R. Perfusing hearts with the NO donor SNAP demonstrated that NO mediated activation of ERK resulted in increased functional recovery in an ex vivo model of I/R[79]. Inhibition of ERK activation via the MEK inhibitor PD98059 results in increased apoptosis in cardiomyocytes exposed to hypoxia followed by reoxygenation, an in vitro mimic of I/R injury [81].

FIG. 3—The MAPK Signaling Pathways in I/R Injury

### Activation of the Different MAPK Pathways Influence Cell Fate During I/R

In the same study, PD98059 treatment also resulted in reduced functional recovery of hearts subjected to ex vivo I/R. In contrast, activation of ERK1/2 in the heart by a number of factors either prior to or during I/R have been shown to provide protection resulting in reduced apoptosis in cell culture models and reduced cell death and improved functional recovery in both ex vivo and in vivo models of I/R [82].

ERK can directly inhibit caspase 3 activation, however; ERK facilitates protection from I/R at a higher level by inhibiting the activity of a number of the pro-apoptotic bcl-2 family members [77, 80]. ERK has been demonstrated to phosphorylate BAD and BAX, allowing 14-3-3 to sequester these proapoptotic proteins in the cytosol, preventing them from translocating to the mitochondria and causing cytochrome c release. ERK activation has also been demonstrated to down regulate the expression of PUMA, another proapoptotic Bcl-2 family member [83]. Deletion of PUMA has been demonstrated to attenuate cardiomyocyte death due to I/R. ERK activation also leads to the upregulation of Mcl-1 which is known to inhibit the activity of proapoptotic Bcl-2 proteins PUMA, BIM, and BAK.

2. JNK

There are three c-jun n-terminal kinase (JNK) encoding genes (JNK1-3), JNK 1 and JNK 2 are expressed ubiquitously while expression of JNK 3 is confined to the brain and testes [84]. Due to alternative splicing, the three JNK genes give rise to 10 different isoforms of JNK. JNK is known to be activated in response to stress but in contrast to ERK, JNK activation, with a few exceptions, has generally been associated with activation of apoptosis and cell death.

JNK is activated by extracellular signals by the ras-raf-cdc42 signal transduction pathway, which, in turn, activate MAP Kinase Kinase Kinase's (MKKK) 1 and 4 as well as ASK1 [78] [85]. The MKKK's, in turn, activate MKK4 and MKK7, resulting in activation of JNK [86]. JNK activation has been reported following I/R and coincides with increased ROS generation [76, 87, 88]. Hypoxia and reoxygenation of neonatal rat ventricular cardiomyocytes (NRVCM's) resulted in increased AK activation and this AK activation could be mimicked through the use of electron transport chain inhibitors that are known to increase mitochondrial ROS generation [89]. Alternatively, in a rat model of myocardial infarction, antioxidant treatment attenuated AK activation in sections of the myocardium that were outside the infarct zone [88]. ASK 1, an upstream activator of MKK4 and MKK7, is inactive under basal conditions due to binding of reduced thioredoxin [85]. Under oxidative stress, such as during I/R, ASK1 becomes activated as the level of reduced thioredoxin is depleted due to a significant increase in cellular oxidants. This ROS-related activation of ASK1 via depletion of reduced thioredoxin also plays a role in activation of the related MAPK, p38 [87].

JNK activation during I/R generally seems to be associated with activation of apoptosis. AK inhibition by AS601245 results in a significant reduction of infarct size in rats subjected to LAD occlusion for 30 min followed by 3 hours of reperfusion [90]. Similar results were found in mice with genetic ablation of either JNK1 or JNK2 or transgenic mice that expressed dominant negative JNK1/2 [91]. Conversely, expression of a constitutively active mutant of MKK7 in adult mice through a very elegant tamoxifen-activated cre-lox system resulted in JNK activation, as well as a progressive cardiomyopathy [92]. AK may contribute to increased apoptosis by destabilizing the 14-3-3 BAD complex which keeps the proapoptotic BAD inactivated and sequestered in the cytosol[93]. Activation of AK is associated with activation and translocation of BAD to the mitochondria. The mechanisms underlying JNK activation of BAD is somewhat controversial with differing reports regarding phosphorylation of BAD directly or phosphorylation of 14-3-3 causing destabilization of the Bad-14-3-3 complex and subsequent activation of apoptosis [93-96]. Jnk has also been shown to directly phosphorylate and inhibit anti-apoptotic Bcl-2 family members [84].

3. p38

Like the other members of the MAPK family, p38 exists as several isoforms [76, 97]. Two of the isoforms, p38α and p38β, are ubiquitously expressed. The third isoform, p38 γ is found in skeletal muscle. The final isoform, p38 δ is found expressed in the kidney, pancreas and small intestine. In the heart it appears that p38α is generally associated with the activation of apoptosis while p38β is linked to hypertrophy and survival signals. The two isoforms are expressed at different levels with more p38α present in the heart than p38β [98].

The upstream kinases MKK3 and MKK6 are responsible for the activation of p38 [99, 100]. MKK3 appears to be a specific activator of p38α while MKK6 is more promiscuous and phosphorylates both forms of p38 found in the heart. p38 activation is complex, with extra cellular signals such as growth factors and inflammatory cytokines able to activate p38 through ASK1 or the TAB1-TAK1 complex [101, 102]. p38 can also be activated via MTK1, MLK3, and ASK1 by stress, such as I/R. The consequences of p38 activation during I/R are extremely controversial, which may be due to an inability to distinguish between activation of the different isoforms.

Activation of p38 during I/R has been shown to induce apoptosis and exacerbate injury in a number of different models. For instance inhibition of p38 via perfusion of pig myocardium with SB203580 results in decreased infarct size following LAD occlusion [103]. Similar observations have been made in rat models of I/R [104] [87]. It has also been demonstrated that p38 activation can reduce contractility of the heart via a negative inotropic effect which can be blocked by expression of dominant negative p38 or administration of SB203580 [105, 106]. Treatment of NRVCM's with the phosphatase inhibitor vanadate, leads to increased cell death following hypoxia and reoxygenation and is associated with increased p38 activation [107]. In this case increased cell death was abrogated by treatment with SB203580.

On the other hand there are studies that demonstrate a protective role for p38 activation. In an ex vivo mouse model of I/R, perfusion with SB203580 prior to I/R resulted in reduced functional recovery and increased tissue damage [108]. Similarly, transgenic mice which over-express the upstream activator MKK6 were found to be protected from I/R injury [98, 109]. Several studies have also found a requirement for p38 activation in ischemic preconditioning [100, 110].

The mechanisms by which p38 activation may protect the myocardium are wide ranging, since p38 seems to participate in a number of diverse pathways. MAPK activated protein kinase 2 (MAPKAPK2) is a downstream target of p38 [111]. Activation of MAPKAPK2 leads to phosphorylation of the small heat shock proteins alpha B-crystallin (αBC) and HSP27. Phosphorylation of both of these proteins is known to provide protection against a number of different stresses. Activation of p38 was also shown to regulate expression levels of αBC [98]. p38 activation is a requirement for NFκB activation as well as IL-6 release in NRVCM's both of which foster protection from stress [112]. Transgenic mice which over-express MKK6 demonstrate a significant reduction in a number of electron transport genes [109]. This results in reduced metabolic activity and reduced ROS generation which may help to protect these mice from FR injury. Activation of p38 has also been shown to be required for insulin, opioid and adenosine receptor mediated protection of the heart as well as beta adrenergic preconditioning [110, 113, 114].

d. Chaperones

Chaperones are a class of proteins whose primary role is to protect proteins from misfolding and aggregating [115]. Most chaperones are stress inducible and fall in to the HSP family of proteins [116]. There are two instances when the presence of chaperones is vital. The first is during folding of newly synthesized polypeptide chains. The exit pore of the ribosome is believed to only be 15 A° wide, limiting protein folding within the ribosome to the creation of α-helices. Therefore, the majority of protein folding occurs either in the cytosol or the lumen of the endoplasmic reticulum where crowding and molecular compaction can result in misfolding and aggregation of the nascent polypeptide before it can establish its native conformation. The presence of chaperones, such as HSP70, chaperonin, and protein disulfide isomerases, prevent this aggregation from occurring by recognizing and binding to exposed hydrophobic residues and unstructured backbone regions.

Chaperones are also required during stress to help proteins maintain their native structure and prevent aggregation. During I/R, oxidative stress and increases in intracellular calcium can lead to protein unfolding and aggregation. Chaperones have been demonstrated to be important in preventing myocardial damage during I/R. Knockout of inducible HSP70 in the heart results in reduced functional recovery and increased creatine kinase release, a marker of necrosis, following ex vivo I/R on a Langendorff apparatus [117]. Knockout of HSP40, a eukaryotic homolog of the prokaryotic DNAJ, results in severe mitochondrial dysfunction that leads to dilated cardiac myopathy in mice, even in the absence of stress [118].

Conversely, overexpression of chaperone proteins results in protection of myocytes from stress induced cell death. Mitochondrial associated HSP70 is one such example, overexpression of HSP70 in NRVCM's results in reduced cell death compared to control cells following simulated ischemia [119]. Further examples of protection from I/R by overexpression exist. For example, overexpression of GRP 78, an ER stress inducible chaperone, has been shown to protect NRVCM's simulated ischemia induced cell death [120]. Likewise, overexpression of either HSP60 or HSP10, components of the chaperonin complex have been shown to protect NRVCM's from apoptosis induced by simulated ischemia[121].

One group of chaperones that have been demonstrated to play a vital role in protecting cardiomyocytes from cell death during stresses, such as I/R, are the family of small heat shock proteins (sHSP's). Small heat shock proteins are a diverse group of proteins; however, they do share several distinctive traits [122]. The members of the family range in size from 12-43 Kd, are stress inducible and generally form large oligomers. The defining characteristic of sHSP's is the presence of the conserved α-crystallin domain. Several studies have demonstrated an important role for sHSP's in protecting cardiomyocytes during stress.

Hearts from transgenic mice which over-express HSP20 demonstrate increased functional recovery and decreased apoptosis in ex vivo Langendorff studies [123]. Similarly, transgenic overexpression of HSP27 in mouse hearts is associated with increased functional recovery and reduced cell death following I/R [124]. The role of another member of the sHSP family of protein, Alpha B-crystallin (αBC), in protecting the myocardium from I/R injury has been firmly established over the last two decades.

### C. Alpha B-Crystallin

Alpha crystallin was first isolated and recognized as one of the major protein components in the lens of the eye in 1927[125]. By the 1970's, alpha crystallin had been found to be composed of the subunits alpha A-Crystallin (aAC) and alpha B-Crystallin and play a role in cataractogenesis[126]. By 1985 both alpha crystallin genes had been cloned[127]. However, it wasn't until 1989 that αBC was reported to be expressed in tissues other than the lens[128]. Besides the lens, αBC is expressed in the lung, brain, kidney, skeletal muscle and the heart. In the heart, αBC is expressed at extremely high levels and is believed to make up as much as 3-5% of the total protein[129]. This discovery led to a flurry of interest in the role of αBC in the heart. Nearly two decades of study regarding αBC and the myocardium have established it as an important cardioprotective protein. The current literature recognizes that αBC acts through many different mechanisms to protect the cardiomyocyte from stress related injury. However, many of the mechanisms of αBC mediated protection remain ill-defined. Here, I review the existing literature regarding αBC in the heart and highlight currently defined mechanisms of αBC mediated cardioprotection.

1. Gene Structure and Regulation of Expression

Alpha B-Crystallin is located on chromosomes 8, 9 and 11 in the rat, mouse and human genome, respectively [130, 131]. In humans, transcription of the αBC gene produces an mRNA of 712 base pairs, which is translated into a 175 amino acid protein with a molecular weight of approximately 22 kilodaltons. The αBC gene is oriented in a head-to-head manner with the related sHSP, HSPB2, with a shared promoter region between the two genes [132]. The promoter region demonstrates preferential activation of αBC transcription in the heart, consistent with the relative expression levels these two genes. Early DNA foot printing experiments identified 5′-enhancer elements within the αBC promoter that are required for expression of αBC in the heart[133]. Further studies have revealed that these elements contain at least one serum response element and two heat shock elements[111].

Although αBC is expressed at very high levels in the heart, very little is understood about the regulation of cardiac αBC gene expression. Studies have identified several transcription factors that have been shown to influence αBC expression. Upstream stimulatory factor (USF) has been shown to bind to the muscle specific enhancer of αBC in nuclear extracts from NRVCM's[133]. The transcription factor HSF1 has been shown to be an important regulator of αBC expression[121]. Hearts from mice lacking the HSF1 gene express 40% less αBC protein compared to control animals. In response to stress it has been shown, both in vitro and in vivo, that activation of MKK6 can result in upregulation of the αBC gene[109, 111]. This may occur through p38 mediated phosphorylation of ATF6. Other transcription factors that have been shown to regulate αBC expression in other cell types but are also found in cardiomyocytes include SP1, glucocorticoid receptor and HMGA1 [134-136].

2. Protein Structure and Function

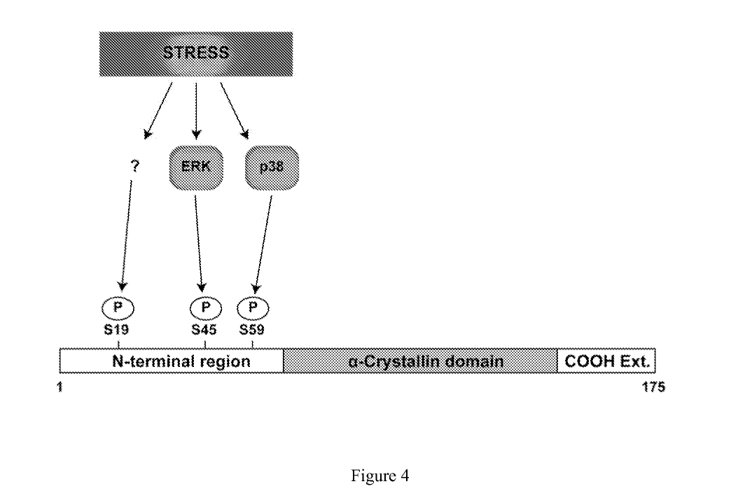

Alpha B-Crystallin protein is considered to have three main regions, the N-terminal region, the core, alpha crystallin domain, and the C-terminal extension (FIG. 4). The core alpha crystallin domain is conserved between sHSP's[122]. Alpha B-Crystallin exists in oligomers ranging in size from 200-800 kilodaltons. In the heart these structures may or may not be homogenous oligomers of αBC. Alpha A-Crystallin is not expressed in the heart however; αBC has been shown to interact with a number of related sHSPs including HSP25/27, HSP22 and HSP20, all of which have been shown to exist as multimers. Studies examining the oligomerization of αBC, using either point mutations or peptide binding studies, have identified sequences within all three regions of the αBC protein that affect oligomerization[137-140]. Oligomerization state can also be affected by post translational modifications, most notably phosphorylation [141, 142]. For instance, mimicking phosphorylation at all three serine residues of αBC is associated with oligomers of a reduced size[143].

FIG. 4—αBC Protein Structure and Phosphorylation by Mapk Pathways

Alpha-BC is a 175 amino acid protein that contains three domains. The N-terminal region contains the three serines that are phosphorylated in response to stress. The α-crystallin domain is a conserved domain present in all small heat shock proteins and is believed to be important for chaperone activity. The c-terminal extension has been demonstrated to be involved in chaperone activity and oligomerization.

Alpha B-crystallin has been demonstrated to be a robust chaperone in vitro for a number of diverse target proteins under several denaturing conditions. Several groups have tried to use point mutation to identify amino acids and sequences within the αBC gene that are required for chaperone activity[139, 144-148]. Point mutations at various sites result in reduced chaperone ability which may suggest that αBC interacts with target proteins at multiple sites. Most of the mutations that affect chaperone activity occur in the c-terminal extension and in the alpha crystallin domain. However, two sites within the N-terminal region stand out for their ability to affect chaperone activity.

Serines 45 and 59 represent two of the three phosphorylation sites on the αBC protein and are known to be phosphorylated via MAPK pathways[111, 149, 150]. The mutation of serines 45 and 59 of αBC to alanine results in a 30% reduction in chaperone activity. Conversely, mimicking phosphorylation of αBC by mutating the serines to aspartate enhances it chaperone ability[143].

3. Post-Translational Modifications

Post translational modifications are changes to the protein that can affect structure and function in either a reversible on non-reversible manner. Many different post translational modifications exist, some of which are controlled enzymatically such as phosphorylation or glycosylation, and some occur non-enzymatically, such oxidation. Several different post translational modifications of αBC have been identified.

Phosphorylation is by far the most well studied of the post translational modifications of αBC. Phosphorylation occurs at serines 19, 45, and 59 in response to various stresses [150]. Stresses such as heat shock or arsenite treatment, result in phosphorylation at all three sites, however, stresses such as H₂O₂ or sorbitol only result in phosphorylation of two of the three sites. This suggested that phosphorylation of each site was controlled by a separate signaling pathway. The ERK pathway was found to be responsible for phosphorylation at serine 45, while the p38 pathway was found to be responsible for phosphorylation of serine 59 [111, 149]. The signaling pathway that results in phosphorylation of serine 19 is currently unknown.

Mimicking phosphorylation via mutation of the serine residues to aspartate or glutamate has provided a wealth of information regarding the consequences of αBC phosphorylation and its effect on αBC structure and function. By utilizing mimics of phosphorylation, serine 59 was identified as being necessary and sufficient for αBC to protect cardiomyocytes from stress [151]. Consistent with these findings are the observation that phosphorylation of αBC serine 59 is increased in the heart in response to I/R [98, 108]. Protection mediated by phosphorylated αBC may be due in part to an enhanced chaperone ability [152-154]. A number of groups utilizing mutants of αBC that mimic phosphorylation at all three sites have demonstrated an enhanced chaperone ability when compared to wild type αBC.

Phosphorylation is a reversible modification that can be used to regulated protein structure and function. The phosphorylation status of any given protein is controlled by the balance of kinase and phosphatase activity. There have not been any phosphatases identified that act specifically on αBC. However, in cardiomyocytes treatment with the phosphatase inhibitor calyculin A was associated with increased αBC phosphorylation and protection from ischemia induced cell death [155].

Several other lesser studied modifications of αBC have also been identified. One interesting modification that may play a role in diabetic associated cardiomoypathy is glycation of αBC. The appearance of advanced glycation end products (AGE) can result as an end product of oxidative modification of proteins[156]. In the lens, accumulation of AGE modified αBC is common with aging and is seen during cataractogenesis [148]. AGE modification of αBC results in a dramatic reduction of chaperone ability which may explain its correlation with the development of cataracts. Diabetics are especially susceptible to AGE-modification of proteins in an around the heart [157]. However, currently there are no reports of AGE modified αBC in the heart

De-amidated αBC has been observed in the heart under basal conditions and rapidly disappears following I/R [139, 158]. Deamidation of glutamine and aspartate within proteins is irreversible suggesting that the disappearance of deamidated αBC is facilitated by rapid degradation of the protein. Deamidation results in reduced αBC chaperone activity which may be why the cell rapidly eliminates deamidated αBC during stress.

O-linked GlcNAcylation of αBC has also been observed in non-stressed rat hearts [159]. Interestingly, glycosylated αBC has never been found to be phosphorylated, and phosphorylated αBC has never been found to be glycosylated. Pulse-chase studies in astroglioma cells found that turnover of the αBC protein was many times slower than turnover of the carbohydrate moiety suggesting that αBC is glycosylated in a reversible manner. These results suggest that glycoslyation may play a role in regulating αBC in some manner, however, there haven't been any reports to demonstrate that this occurs in the heart.

4. Protection of the Heart During ER

Alpha B-Crystallin has emerged as an important protector of the myocardium during stress. In nearly every report concerning αBC, the heart, and I/R injury, αBC has been shown to promote myocyte survival and preservation of cardiac function. While a complete understanding of the mechanisms of αBC mediated protection is elusive, several mechanisms have been identified and described.

a. Functional Studies in Mouse Models

Some of the most compelling evidence for a cardioprotective role for αBC comes from studies that have utilized transgenesis and knockout technology to create mouse models that facilitate study of the whole heart. The first published study to use such a model came from Wolfgang Dillmann's lab, where they created a line of mice that over-expressed wildtype αBC [160]. These transgenic mice expressed roughly 7 times more αBC protein in the heart compared to control animals. Using an ex vivo working heart model, hearts from both control and transgenic animals were exposed to I/R injury. The hearts of transgenic mice demonstrated a significantly better functional recovery as well as reductions in infarct size and apoptosis compared to control hearts.

A compliment to the αBC transgenic study was published by our lab shortly thereafter [161]. This study utilized αBC knockout mice that had originally been derived by Eric Wawrousek's lab in order to dissect the roles of aAC and αBC in the lens of the eye [162]. Mice with a deletion of the αBC gene develop normally however, they do die prematurely due to malnutrition primarily due to development of megaesophagus around 10 months of age. The heart, however, appears normal up till and including death. Hearts from these animals were subjected to I/R using an ex vivo Langendorff perfusion apparatus. Hearts from knockout animals showed significantly reduced functional recovery compared to hearts from control animals. This reduced functional recovery coincided with significantly higher levels of apoptosis and necrosis in the knockout animals when compared to hearts from control animals.

One other αBC mouse model has been generated and provides an insight in to the importance of αBC's function in the heart. The existence of the R120G mutation of αBC was first brought to light in a genetic analysis of a French family with a large number of cases of cardiomoypathy and cataracts [163]. Follow up studies on the effect of R120G on αBC's structure and function found that this mutation inhibited chaperone activity and resulted in the formation of desmin rich aggresomes.

Mice harboring the R120G mutation recreate the phenotype seen in the family members, where the mutation was originally described [164]. R120G mice demonstrate aggregate formation within myocytes accompanied by gross disorganization of the myofilaments. Cardiac function was significantly compromised and by 6 months the mice were in the advanced stages of heart failure. Taking into account results from in vitro studies that demonstrate that recombinant αBC-R120G is severely compromised in its chaperone activity, these results suggests that the chaperone activity of αBC is vital to maintaining homeostasis within the cardiomyocyte [165].

b. Translocation to the Sarcomeres in Response to I/R

Translocation of αBC out of the cytosol in response to stress was first reported in guinea pigs hearts subjected to ischemia [166]. The hearts were fractionated to produce a soluble and insoluble pellet. The insoluble pellet contained the cytoskeletal and sarcomeric proteins. Under basal conditions αBC was found exclusively in the cytosol but following ischemia αBC was detectable in the insoluble fraction. A similar phenomenon has been reported in several different cell lines in response to heat stress [167].

These studies, while intriguing, where only a small step in determining targets for αBC during stress. However, they did provide a clue as to where to start looking for targets of αBC. Golenhofen et al. were the next to address translocation of αBC out of the cytosol, this time using left coronary artery occlusion (LAD), an in vivo model of myocardial infarction, in pigs [168]. Golenhofen et al. utilized immunocytofluoresence to visualize the location of αBC under basal and stress conditions. Under basal conditions αBC was cytosolic and showed very little association with the cytoskeleton or sarcomeres. However, following LAD there was a striking increase in αBC association with the z-lines of the sarcomeres. Two dimensional gel analysis of tissue samples demonstrated the appearance of acidic forms of αBC following LAD suggesting that translocation of αBC to the sarcomeres coincided with phosphorylation of αBC. Many studies have followed that examine the interaction of αBC with sarcomeric proteins, in particular proteins that make up the z-lines[168-173]. To date αBC has been shown to interact with actin, myosin, desmin, titin and vimentin.

The protective effect of αBC translocation to the sarcomere was analyzed using papillary muscles from the hearts of αBC knockout mice[174]. Under basal conditions the papillary muscles from αBC knockout and control mice produced equivalent contractile force. In response to ischemia the papillary muscles of αBC mice went in to contracture much sooner and experienced much higher tensile force during contracture when compared to control animals. These results demonstrated an increased stiffness in αBC knockout mice in response to stress.

Elasticity within the sarcomeres is maintained, in part, by the structural support of titin[175]. Titin spans half the distance of a sarcomere, connecting the m-line and z-disc. Titin, as would be expected for the largest known protein, has a complex structure, the region of titin that spans the I-band has a number of globular domains with no well-defined secondary structure. The regions can stretch and unfold, but by maintaining interactions within a globular structure, these regions provide elasticity to the sarcomeres.

Alpha B-Crystallin was shown by electron microscopy using immunogold labeling to localize to the I-band portion of the sarcomeres [171]. Binding assays using recombinant fragments of the different regions of titin confirm that αBC binds to the globular regions of titin. Together these results suggest that αBC may help maintain sarcomeric elasticity during stress by binding to the globular regions of titin [169]. By maintaining sarcomeric elasticity αBC helps maintain sarcomeric structure during ischemia when sustained unregulated contraction applies mechanical strain to neighboring sarcomeres. This data is further supported by electron micrographs of the sarcomeres of αBC knockout mice which demonstrate larger I-band regions when compared to control animals, suggesting that the sarcomeres are being stretched apart [161].

c. Anti-Apoptotic Effects

The old axiom that the heart is a terminally differentiated organ and that there is no turnover of myocytes, has gone by the wayside. However, hearts that experience severe I/R injury generally replace dead myocytes with scar tissue and eventually progress to failure. As such, it appears that the regenerative ability of the native stem cell population within in the heart is insufficient to replace the large numbers of myocytes lost to a major ischemic injury. In order for the heart to demonstrate functional recovery following an ischemic event, and avoid failure in the future, there must be a preservation of the myocyte population. Cell death as a consequence of I/R injury occurs through both necrotic and apoptotic pathways. Direct inhibition of apoptosis using caspase inhibitors has been shown to reduce infarct size in in vivo models of I/R [176]. This suggests that inhibiting apoptosis may be an effective strategy for preserving heart function following I/R. In response to I/R, the heart activates a number of anti-apoptotic signaling pathways, such as PI3K/AKT, PKCε, ERK, and p38. Alpha B-crystallin lies downstream of both ERK and p38 and has been demonstrated to be an anti-apoptotic protein.

Overexpression of wildtype αBC, and especially αBC phosphorylated on serine 59 in various cell culture models prevents apoptosis due to oxidative, hyperosmotic, and hypoxic stresses [151]. Transgenic mice that over express αBC were found to have fewer TUNEL positive cells than control animals following I/R [160]. Conversely, hearts from αBC knockout mice exposed to I/R had increased DNA laddering compared to control hearts [161]. Examination of the mechanisms by which αBC confers protection from apoptosis demonstrate it interacts with and inhibits the activity of several pro-apoptotic proteins.

In response to stress proapoptotic members of the Bcl-2 family such as Bad, Bax, Bim, and Bcl-Xₛ translocate to the mitochondria where they induce apoptosis by facilitating release of cytochrome c from the mitochondria. Overexpression of αBC inhibited translocation of both Bax and Bcl-Xₛ from the cytosol to the mitochondria in response to staurosporine treatment [177]. Furthermore, it was shown that overexpression of αBC was sufficient to prevent caspase 3 activation and DNA laddering following staurosporine treatment Alpha B-crystallin was shown by immunoprecipitation from lens epithelial cell extracts to interact with the pro-apoptotic Bcl-2 family members Bax and Bcl-Xₛ. These results suggest that αBC can prevent apoptosis at the level of the mitochondria by preventing cytochrome c release perhaps by keeping the mitochondrial permeability transition pore closed. Consistent with these results are reports that demonstrate incubating recombinant αBC with isolated mitochondria prevent calcium induced swelling, and that overexpression of αBC in NRVCM's helps maintain mitochondrial membrane potential during oxidative stress.

In addition to preventing apoptosis at the level of the mitochondria, αBC can also prevent-receptor mediated activation of apoptosis through inhibiting TRAIL signaling [178]. More interestingly however, is the ability to prevent pro-caspase 3 maturation that was first observed in differentiating myoblasts [179]. In a follow up study it was shown that αBC can prevent caspase 3 activation, regardless of whether apoptosis was activated by the intrinsic or extrinsic pathways[180]. This study also found that pro-caspase 3 co-immunoprecipitated with αBC suggesting that caspase 3 maturation is inhibited by direct interaction with αBC. Taken together, these studies suggest that αBC may be able to regulate apoptosis from the earliest apoptotic signals all the way through the end events of cell death.

The literature clearly demonstrates that αBC plays a role in protecting the myocardium from FR injury. It does this through a number of different mechanisms that help to preserve cell structure and function, while also directly inhibiting cell death. However, several observations suggest that there may be a number of unexplored mechanisms by which αBC provides protection against I/R.

Expression of related sHSP's such as HSP25/27 have been shown to influence redox status in a number of different cell lines. Expression of αBC in L929 fibroblasts was shown to provide protection against hydrogen peroxide but the mechanism of protection was never explored. Hearts from mice over expressing αBC, that were submitted to I/R injury, were found to contain significantly less malondialdehyde, a product of lipid peroxidation, when compared to control animals. Hearts from mice which lack the transcription factor, HSF1, were found to express 40% less αBC and were found to have a lower reduced glutathione to oxidized glutathione ratio, when compared to hearts from control animals. However, given this collection of observations, an analysis of the effects of αBC expression on the redox status of the heart, and the mechanisms by which αBC may influence redox status has never been performed.

Alpha B-crystallin has been demonstrated to be a potent inhibitor of apoptosis, and has been shown to inhibit apoptotic signaling at several points within the apoptotic signaling pathways. This includes, interactions with proteins that translocate to the mitochondria to induce apoptosis such as Bax and BclXₛ, and effector proteins that are activated by events regulated by the mitochondria such as caspase 3. Recently, several studies form our own lab, have demonstrated that αBC translocates to the mitochondria during I/R and that its presence there may inhibit events that lead to apoptosis. However, these studies have not yet examined the nature of the translocation event, the targets of αBC at the mitochondria, or the mechanism by which the presence of αBC at the mitochondria may affect apoptotic signaling.

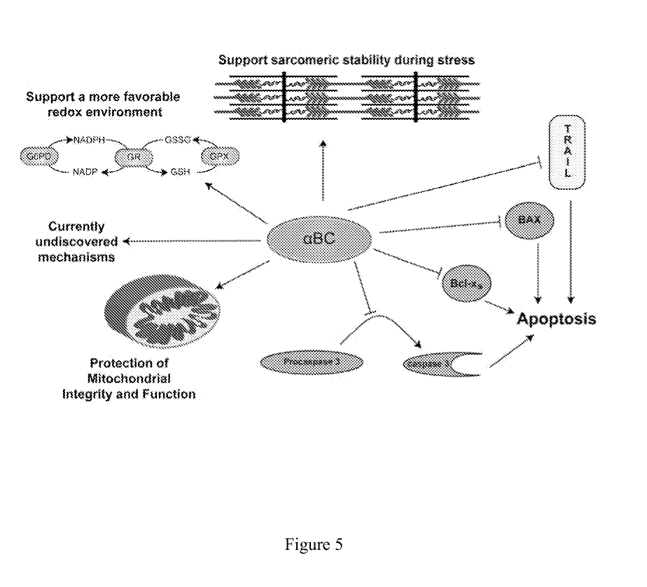

FIG. 5 illustrates an alternative embodiment: Alpha B-crystallin is an important protective protein within the myocardium and acts in several different ways to preserve the structure and function of the heart. The mechanisms of protection by αBC include, but are not limited to, stabilizing sarcomeric and cytoskeletal structure, preventing apoptosis, maintaining a favorable redox environment, and protecting the integrity and function of the mitochondria. While the invention is not limited by any particular mechanism of action, in an alternative embodiment the following is an alternative mechanism of action of this invention:

Alpha B-crystallin is an important protective protein within the myocardium and acts in several different ways to preserve the structure and function of the heart. The mechanisms of protection by αBC include, but are not limited to, stabilizing sarcomeric and cytoskeletal structure, preventing apoptosis, maintaining a favorable redox environment, and protecting the integrity and function of the mitochondria, as illustrated in FIG. 5.

This alternative embodiment will be addressed in the following three chapters. The first chapter will address the mechanisms by which αBC may prevent apoptotic signaling by interacting with the mitochondria; noting that the invention is not limited by this or any particular mechanism of action. The second chapter explores the effect of αBC on the redox status of the heart and demonstrates how αBC can influence intracellular glutathione levels. The third chapter introduces a novel protein transduction tool of this invention that can be utilized as a delivery vehicle for the use of αBC as a therapeutic for treating MI. This can be used in the development for a novel drug delivery platform that can not only to deliver αBC but also to deliver other protein and small molecule drugs.

### Chapter Two

### 1. Introduction

Ischemia/reperfusion (I/R) in the heart leads to the generation of mitochondrial-derived reactive oxygen species (ROS), which can contribute to myocardial damage [8, 13]. A number of signaling cascades are activated in the heart during I/R, including the mitogen activated protein kinases (MAPKs), which have been associated with both tissue damage and protection [77, 80, 102, 103, 110, 112, 181-184]. Activation of p38 MAPK leads to the phosphorylation of numerous proteins, including the small heat shock protein (sHSP), alpha B-crystallin (αBC), one of the ten-member sHSP family of molecular chaperones [111, 149]. αBC is expressed in several tissues, including the lung, eye, brain, and skeletal muscle; however, its expression in the heart is particularly noteworthy, since it comprises 3-5% of the total protein in the myocardium [122, 128, 170].

Numerous studies have established a role for αBC in protecting the myocardium from I/R. In response to stress αBC is phosphorylated on serine-59, in a p38 dependent manner. Mimicking the phosphorylation of αBC at serine-59 was shown to enhance the ability of αBC to protect cells against several different stresses that imitate I/R [151]. Like other members of the sHSP family, it is through its molecular chaperone activity that αBC is thought to exert most of its protective functions [168, 178-180]. Because of its cardioprotective effects, identifying the cellular binding partners of αBC and examining the functional consequences of αBC binding to those targets, have been the emphases of numerous studies; in fact, it has been proposed that post-translational modification of αBC, specifically phosphorylation, may augment its chaperone activity [185].

Transgenesis and gene-targeting have provided additional evidence that αBC plays an important protective role in the mouse heart, in vivo. For example, it was demonstrated that compared to wild type mouse hearts, the hearts from transgenic mice that over-express αBC in a cardiac-specific manner exhibited less tissue damage and preserved contractile function when subjected to ex vivo I/R [160]. It was also shown that when subjected to ex vivo I/R, the hearts from mice lacking αBC were more susceptible to injury than hearts from wild type mice [161].

In terms of its subcellular localization, αBC is very dynamic. Several studies have demonstrated that in the heart, stress induces αBC to translocate from a diffuse cytosolic locale to sarcomeres, where has been hypothesized to stabilize myofilament structure [167-169, 186]. In mouse hearts subjected to ex vivo I/R, αBC translocates to mitochondria, where it was postulated to bind to key mitochondrial proteins and to preserve mitochondrial function [98, 108]. Moreover, the level of phospho-αBC-S59 associated with mitochondria was increased upon I/R. Inhibiting p38, which decreased mitochondrial phospho-αBC-S59, also increased myocardial damage in an ex vivo model of I/R. These findings support the alternative embodiment that p38, which is activated in the heart during ischemia and reperfusion, leads to increased accumulation of phosphorylated αBC in mitochondrial fractions, and that the resulting mitochondrial phospho-αBC-S59 may exert a protective function by interacting with and affecting the function of specific mitochondrial proteins. The results of the present study are consistent with this alternative embodiment, and they suggest that mitochondrial αBC may interact with the voltage-dependent anion channel (VDAC), which resides on the outer mitochondrial membrane and plays a central role in preserving mitochondrial function [187-189]. Furthermore, consistent with maintaining mitochondrial function during FR, αBC may also interact with key components of the electron transport chain.

### 2. Materials and Methods

A. Animals—Approximately 100, 10-14 week-old C57/BL6 mice (Mus musculus) were used in this study. All procedures involving animals were performed in accordance with institutional guidelines. The animal protocol used in this study was reviewed and approved by the San Diego State University Institutional Animal Care and Use Committee.
B. Ex Vivo Ischemia-Reperfusion—Global no-flow ex vivo I/R was performed on a Langendorff apparatus, as previously described [161]. Briefly, mice were treated with 500 U/kg heparin 10 min prior to administration of 150 mg/kg of pentobarbital, both via intraperitoneal injection. Animals were then sacrificed, hearts were quickly removed and placed in ice-cold modified Krebs Henseleit buffer. The aorta was then cannulated and the heart was mounted on a Langendorff apparatus and perfused with oxygenated Krebs Henseleit buffer at a constant pressure of 80 mmHg. The left atrium was removed and a water-filled balloon connected to a pressure transducer was inserted into the left ventricle and inflated in order to record left ventricle developed pressure (LDVP). Hearts were electrically paced at 8.7 to 9.2 Hz via an electrode placed on the right atrium. Following a 30-minute equilibration period, hearts were subjected to varying times of ischemia±subsequent reperfusion. Hearts were submerged in buffer at 37° C. at all times. Following the appropriate treatment times, hearts were quickly removed from the apparatus, the right atria and any remaining vessels and connective tissue were removed, and hearts were flash frozen in liquid nitrogen. Hearts were stored at −80° C. until processed.
C. Preparation of Subcellular Fractions—Frozen hearts were pulverized while in dry ice and then homogenized in 1 ml of isolation buffer (70 mM sucrose, 190 mM mannitol, 20 mM HEPES, 0.2 mM EDTA, 200 μM sodium orthovanadate, 10 μg/ml aprotinin, 10 μg/ml leupeptin, 0.5 mM PNPP, 1 mM PMSF). Following homogenization 70 μl of the homogenate were added to 400 μl of RIPA buffer (150 mM NaCl, 20 mM Tris-HCl pH 7.5, 0.1% SDS, 1% Triton X-100, 1 mM EDTA, 200 μM sodium orthovanadate, 10 μg/ml aprotinin, 10 μg/ml leupeptin, 0.5 mM PNPP, 1 mM PMSF), this whole homogenate fraction was saved for immunoblot analysis. The remainder of each homogenate was centrifuged at 600×g for 10 minutes to remove nuclei and myofibrils. The resulting supernatant, containing mitochondria and membrane fractions, was centrifuged at 5,000×g for 15 min. producing a pellet containing the mitochondria and a supernatant containing the cytosol and the endoplasmic reticulum. The pellet was washed twice with 1 ml of isolation buffer and resuspended in 400 μl of RIPA buffer to produce the mitochondrial fraction. The supernatant was centrifuged at 100,000×g for 60 min. The resulting supernatant was the cytosolic fraction.
D. Mitochondrial Trypsin Protection Assay—Mitochondria were isolated as described above; 200 μg of isolated mitochondrial protein were resuspended in 200 μl of isolation buffer and treated±trypsin (1 mg/ml) for one hour on ice. Mitochondria were then collected by centrifugation at 5,000×g for 15 min, washed twice with isolation buffer containing soybean trypsin inhibitor (1 mg/ml), and then resuspended in 100 μl of RIPA buffer, also containing soybean trypsin inhibitor (1 mg/ml).
E. Cytochrome C Release Assay—Neonatal rat ventricular myocyte cells (NRVMC) were cultured, as previously described [111]. NRVMC were infected with recombinant adenovirus strains encoding either no αBC (control), wild type αBC, or a mutant form of αBC which mimics phosphorylation at serine 59 while blocking phosphorylation at serines 19 and 45 (αBC-AAE). Forty eight hours after infection, cultures were placed in minimal media containing 200 μM H₂O₂ and incubated for one hour, after which cells were harvested by trypsinization, collected by centrifugation, and mitochondria isolated, as described above.
F. Immunoprecipitation—NRVCM's were infected with recombinant adenovirus encoding αBC-AAE with an N-terminal HA tag. Forty eight hours after infection, cultures were exposed to 200 μM H₂O₂ in minimal media for 1 hour. Cells were released from culture dishes by trypsinization, collected by sedimentation, and fractionated by differential centrifugation, as described above, to obtain mitochondrial-enriched fractions. Mitochondria were then homogenized in RIPA buffer, as described above. For each immunoprecipitation, 30 μg of mitochondrial protein were incubated for approximately 16 h at 4° C. in a total volume of 500 μl with either HA antibody, or control mouse IgG antibody, both conjugated to agarose beads. Following incubation, beads were washed twice with PBS-T (PBS with 0.05% Tween-20), followed by 3 washes with PBS. Antibody-protein complexes were released from the agarose beads by boiling samples in SDS sample buffer. Samples were then fractionated by SDS-PAGE and subjected to immunoblotting for αBC, adenine nucleotide translocase (ANT) or voltage-dependent anion channel (VDAC).

Immunoprecipitations from mouse heart mitochondria were performed in a similar manner however, endogenous protein was immunoprecipitated using sepharose beads and anti-αBC antibody.

G. Proteomic analysis (MudPIT)—Proteomic analysis of immunoprecipitation samples was performed as previously described [109].

### 3. Results

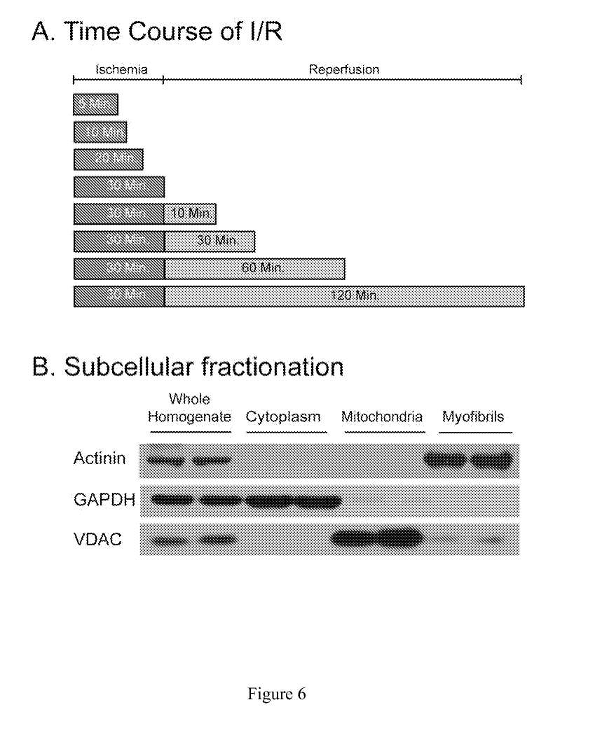

To examine the kinetics of αBC movement to mitochondria, mouse hearts were subjected to ex vivo ischemia (I) or ischemia/reperfusion (I/R), for various times (FIG. 6A), followed by homogenization and subcellular fractionation. Immunoblotting for the myofibril, cytosol and mitochondria marker proteins, α-actinin, GAPDH, and VDAC, respectively, in subcellular fractions prepared from a control heart showed that the mitochondrial fractions were free from contamination from cytosolic and myofibril proteins (FIG. 6B), which can potentially contain large quantities of αBC.

FIG. 6 illustrates a diagram of the I/R protocol used in this study and analysis of the subcellular fractionations for organelle contamination. Panel A—Following 30 minute of equilibration mouse hearts were subjected to various times of ischemia and reperfusion on a Langendorff apparatus (n=3 mouse hearts/time) as shown. A continual perfusion control that matched each I and I/R time was also generated. Panel B—After each treatment, hearts were homogenized and, after removal of a sample for later analysis, homogenates were subjected to subcellular fractionation. The tissue was later homogenized and fractionated by differential centrifugation and examined by immunoblot for typical subcellular fraction marker proteins.

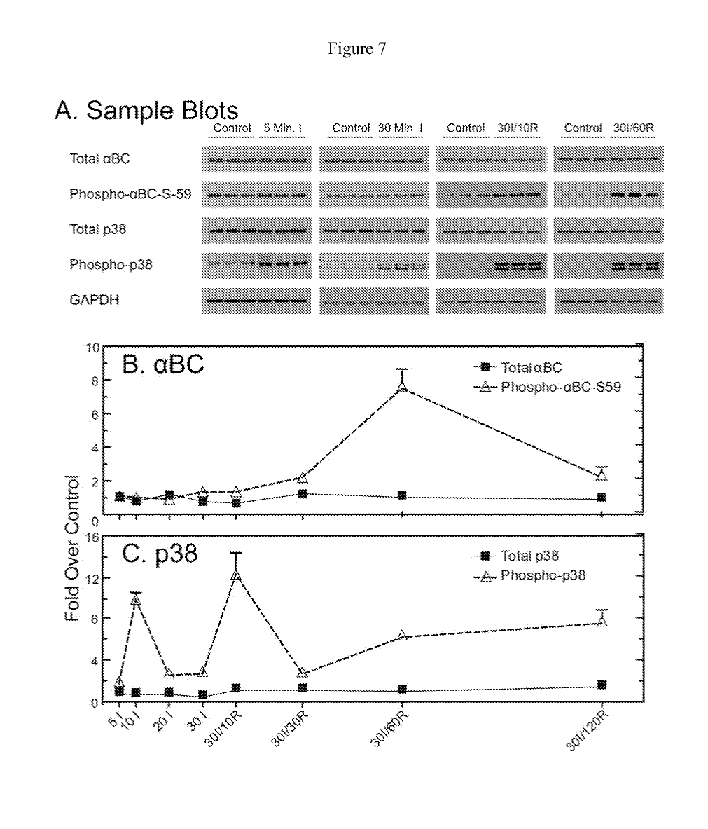

Whole heart homogenates were examined by immunoblotting; examples of blots prepared from homogenates of hearts subjected to 5 or 30 min of I, or to 30 min of I followed by 10 or 60 min of R are shown in (FIG. 7A). An antibody that cross-reacts with all forms of αBC was used to measure total αBC in homogenates. Compared to control hearts, the levels of total αBC in hearts subjected to I or I/R did not change significantly (FIG. 7A, Total αBC). Quantification of immunoblots performed at all of the times shown in FIG. 1A further demonstrated that compared to perfusion time-matched controls, there was no significant loss of αBC after any of the I or I/R times (FIG. 7B, Total αBC, squares). In contrast, antibodies specific for αBC phosphorylated on serine-59 (phospho-αBC-S59) showed that the level of phospho-αBC-S59 in whole heart homogenates reached a maximum of 8-fold over control after 30 min of I followed by 60 min of R (FIG. 7A, Phospho-αBC-S59; FIG. 7B, triangles). Since the phosphorylation of αBC on serine-59 depends on p38 activation, the levels of total and phosphorylated p38 (P-p38; active) were also examined. As expected, total p38 did not change as a function of I/R (FIG. 7A, total p38; FIG. 7C, total p38 squares). However, the relative levels of phospho-p38 increased transiently, to about 10-fold over control within 10 min of I, and then decreased to about 3-fold over control at the longer I times (FIG. 7C, Phospho-p38 triangles).

FIG. 7—Effect of Ischemia or Ischemia/Reperfusion on αBC, Phospho-αBC-S59, p38 and Phospho-p38 in Whole Heart Homogenates

Prior to subcellular fractionation, samples of the whole heart homogenates from the 8 experimental samples shown in FIG. 6A, and 8 time-matched controls (n=3 hearts per time), were subjected to immunoblot analysis.

Panel A—Samples from 4 of the time points were analyzed by immunoblotting for Total αBC, Phospho-αBC-S59, p38, Phospho-p38 and GAPDH.
Panel B—The immunoblots of all samples for Total αBC (squares) and Phospho-αBC-S59 (triangles) were quantified; values are shown as fold over control hearts for each time point normalized to GAPDH.
Panel C—The immunoblots of all samples for Total p38 (squares) and Phospho-p38 (triangles) were quantified; values are shown as fold over control hearts for each time point normalized to GAPDH. All values=Mean±SEM, n=3 for each time point. All values for phospho-αBC-S59 and phospho-p38 are significantly greater than control perfusion values (p<0.05).

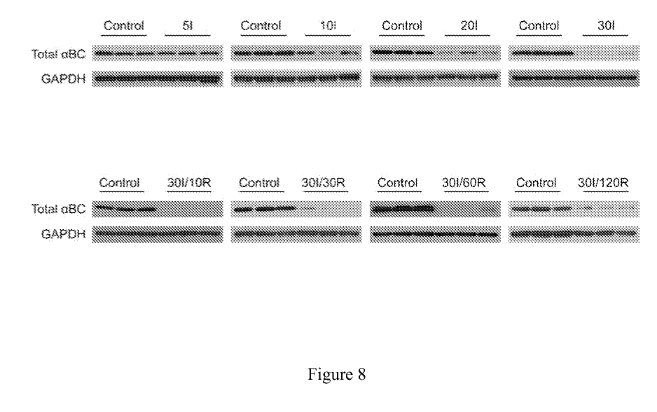

Phospho-p38 exhibited a second transient increase of 12-fold over control after 301/10R, which was followed by more modest increases to about 6- and 8-fold over control after 60 and 120 min of R, respectively. This profile of p38 activation is consistent with the transient activation of p38 during ischemia and reactivation again during reperfusion, the latter of which requires mitochondrial-derived ROS. These results demonstrate that αBC phosphorylation on serine-59 occurred primarily during R when whole heart homogenates were examined; however, dramatically different results were observed in the subcellular fractions. For example, αBC left the cytosol rapidly following the onset of I, by 30 minutes of I there was in a complete loss of total αBC and phospho-αBC-S59 (FIG. 8, 5-30 Min I). This loss of αBC persisted throughout all reperfusion times examined (FIG. 8, 30I/10-120 Min. R). Thus, during I, αBC quantitatively moved from the cytosol to other subcellular fractions and remained at these other cellular locations throughout the reperfusion time points examined.

FIG. 8—Effect of ischemia and ischemia/reperfusion on αBC in cytosolic fractions. Immunoblotting was performed on cytosolic fractions from all the time points to examine total αBC and GAPDH.

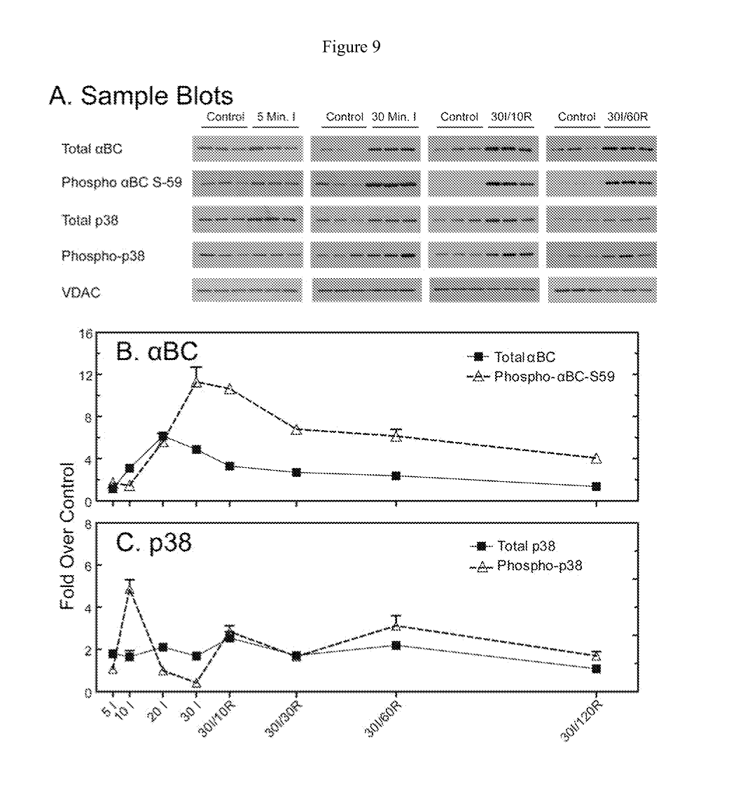

We recently reported that phospho-αBC-S59, plays important roles in protecting mitochondria during stress. However, after examining the whole heart homogenates in the current study, it appeared that αBC phosphorylation might occur too late to provide significant protection of mitochondria from reperfusion damage. Accordingly, mitochondrial fractions were examined. Examples of blots prepared from hearts subjected to I or I/R as in FIG. 2A are shown in FIG. 9A. In contrast to whole homogenates, total αBC in mitochondrial fractions (total αBC) increased primarily during I (FIG. 9A, Total αBC). Quantification of sample blots from all times shown in FIG. 1A demonstrated that total mitochondrial αBC increased by about 4-fold after as little as 10 min of I, reaching a maximum of 7-fold over control by 20 min of I and subsequently declining throughout the remaining times of I and I/R (FIG. 9B, Total αBC squares). The level of mitochondrial phospho-αBC-S59 was also maximal during I, but exhibited delayed kinetics compared to that of total mitochondrial αBC (FIGS. 9A, and 9B, P-αBC-S59 triangles). In fact, mitochondrial phospho-αBC-S59 was maximal after 30 min of I, 10 min after the maximum for total mitochondrial αBC. At longer R times, the level of mitochondrial phoshpo-αBC-S59 declined steadily to about 4-fold over control after 120 min of R, the last time point examined (FIG. 4B, Phospho-αBC-S59).

FIG. 9—Effect of ischemia and ischemia/reperfusion on αBC, Phospho-αBC-S59, p38, and Phospho-p38 in mitochondrial fractions: After subcellular fractionation, samples of the mitochondrial fractions from the 8 experimental samples shown in FIG. 1A, and 8 time-matched controls (n=3 hearts per time), were subjected to immunoblot analysis. Panel A—Samples from 4 of the time points were analyzed by immunoblotting for Total αBC, Phospho-αBC-S59, p38, Phospho-p38 and VDAC. Panel B—The immunoblots of all samples for Total αBC (squares) and Phospho-αBC-S59 (triangles) were quantified; values are shown as fold over control hearts for each time point normalized to VDAC. Panel C—The immunoblots of all samples for Total p38 (squares) and Phospho-p38 (triangles) were quantified; values are shown as fold over control hearts for each time point normalized to VDAC. All values=Mean±SEM, n=3 for each time point. All values are significantly greater than control perfusion values (p<0.05).

Since it appeared as though αBC phosphorylation occurred after translocation to mitochondria, and since p38 activation is required for this phosphorylation, the levels of p38 in the mitochondrial fractions were analyzed. Total p38 in mitochondrial fractions increased by about 2-fold over control within 5 min of the onset of I (FIGS. 9A and 9C, Total p38, squares). Total mitochondrial p38 remained at this level through 60 min of R, and then declined somewhat thereafter. The levels of phospho-p38 in the mitochondrial fractions exhibited a transient increase to about 5-fold over control within 10 min of I, which was followed by a decline to nearly control levels of phosphorylation at the later I times (FIG. 9A, Phospho-p38; FIG. 9B open triangles). Mitochondrial phospho-p38 levels increased to about 2.5-fold over control upon 10 min of R, and remained at this level through 60 min of R, and then declined to control levels by 120 min or R. Taken together, these results indicate that in contrast to the whole heart homogenates, the increases in mitochondrial total αBC and phospho-αBC-S59 occurred primarily during I. Moreover, the timing of the increases in phospho-p38 in mitochondrial fractions provided support for the idea that αBC is phosphorylated in a p38-dependent manner, and that at least in part, this phosphorylation takes place after its translocation to mitochondria.

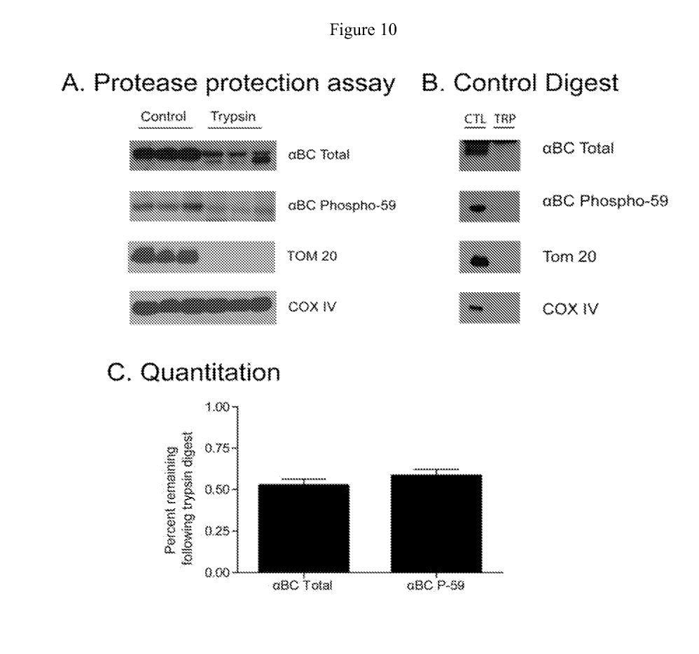

FIG. 10—Effect of Trypsin on Mitochondrial αBC: Hearts (n=3) were subjected to 30 min of ex vivo ischemia followed by 10 minutes of reperfusion, and then homogenized and mitochondria isolated as described in Materials and Methods. 200 μg of isolated mitochondrial protein were then treated with 1 mg/ml trypsin for 1 hour at 4° C. Panel A—Immunoblots for Total αBC, Phospho-αBC-S59, TOM20 and COX4 before and after incubating isolated mitochondria with trypsin. TOM20 was used as a marker for proteins located outside the mitochondrial outer membrane and COX4 was used as a marker for proteins located within the outer mitochondrial membrane. Panel B—Immunoblots for Total αBC, Phospho-αBC-S59, TOM20 and COX4 after mitochondrial membrane disruption by sonication before trypsin treatment. Panel C—Quantitation of the fraction of Total αBC, Phospho-αBC-S59. COX4 and TOM20 remaining after trypsin treatment of intact mitochondria. Values are represented as mean percent remaining±SEM, n=3.

To examine the nature of the association of αBC with mitochondria, a protease protection experiment was carried out. After treatment of isolated mitochondria with trypsin, total αBC and phospho-αBC-S59 were decreased, but only by about 50% (FIGS. 10A and C), suggesting that about half of the αBC may be localized to regions of mitochondria that are protected from trypsin. In contrast, TOM 20, a protein that resides on the cytoplasmic face of mitochondria, was completely digested by trypsin, while COX4, which resides in the mitochondrial matrix, was completely resistant to protease treatment (FIG. 10A, TOM20 and COX IV). To address the formal possibility that a certain portion of αBC could not be degraded by trypsin, mitochondrial integrity was compromised by sonication prior to protease treatment, and it was found that αBC, phospho-αBC-S59 and COXIV were completely degraded. These results suggest that a portion of the mitochondrial αBC and phospho-αBC-S59 reside on the surface, while the remainder may be localized within the outer mitochondrial membrane.

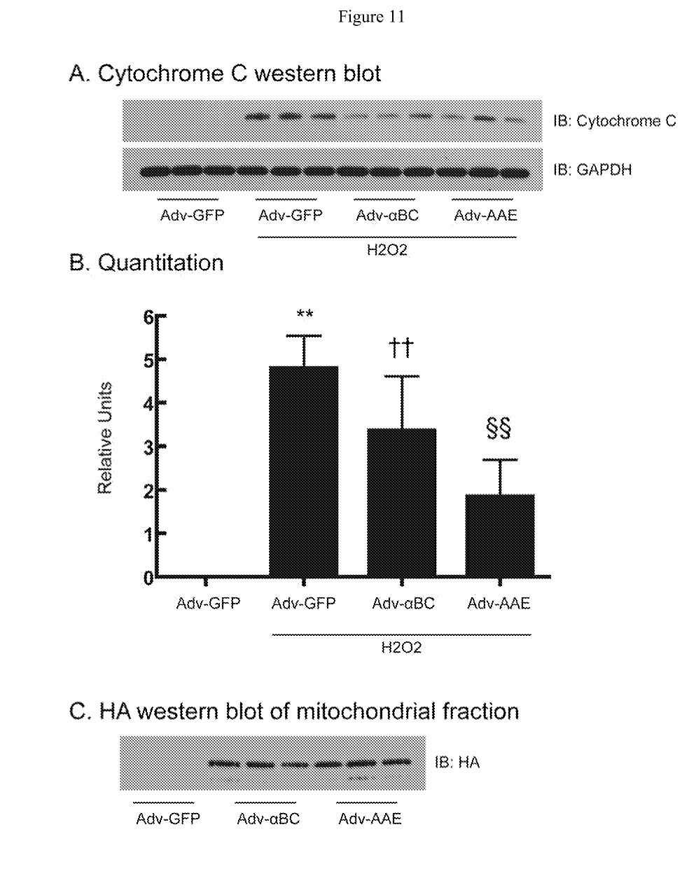

FIG. 11—Effect of wild type αBC-AdV and αBC-AAE-AdV on H₂O₂-induced Cytochrome C release: Overexpression of wild type αBC or the phosphorylation mimic, αBC-AAE, in neonatal rat ventricular cardiomyocytes was accomplished via infection with adenovirus (AdV) harboring the appropriate expression construct, or no αBC expression construct (Con-AdV). Cells were treated with 200 μM H₂O₂ for 1 hour, followed by subcellular fractionation and cytochrome c immunoblots on the cytosolic fraction. Panel A—Immunoblots of cytochrome c and GAPDH in the cytosol. Panel B—Quantitation of cytochrome c in cytosol normalized to GAPDH. Values represent average±SEM, ** and †† are different from other groups by ANOVA with Student Newman Keuls post hoc test, p<0.05. Panel C—Immunoblot for HA-tagged, AdV-encoded wild type αBC, or αBC-AAE in mitochondrial fractions isolated from cultured cardiac myocytes.

To examine possible functional roles for mitochondrial αBC, the effects of over expressing wild type αBC, or αBC-AAE, the latter of which mimics phosphorylation at serine-59, in cultured cardiac myocytes were assessed. When cultures were infected with the control adenovirus, Con-AdV, but not subjected to oxidative stress, there was no detectible release of mitochondrial cytochrome c, as expected (FIG. 11A, lanes 1-3; FIG. 11B, bar 1). However, when Con-AdV-infected cultures were treated with H₂O₂, the release of mitochondrial cytochrome c increased dramatically (FIG. 11A, lanes 4-6; FIG. 11B, bar 2). In contrast, when cultures were infected with αBC-AdV or αBC-AAE-AdV, H₂O₂-activated cytochrome c release decreased by about 25 and 50%, respectively (FIG. 11A, lanes 7-12; FIG. 11B, bars 3 an 4), consistent with the alternative embodiment that mitochondrial αBC protects mitochondria from oxidative damage, and that phosphorylation of αBC on serine-59 may enhance these protective effects. These results are also consistent with our previous findings that αBC-AdV or αBC-AAE-AdV both reduced H₂O₂-mediated loss of mitochondrial membrane potential in NRVMC[108]. Immunoblots showed that HA-αBC and HA-αBC-AAE were both associated with mitochondria (FIG. 11C), consistent with the alternative embodiment that while the association of non-phosphorylated αBC with mitochondria may provide some protection, phospho-αBC-S59 provides maximal protection.

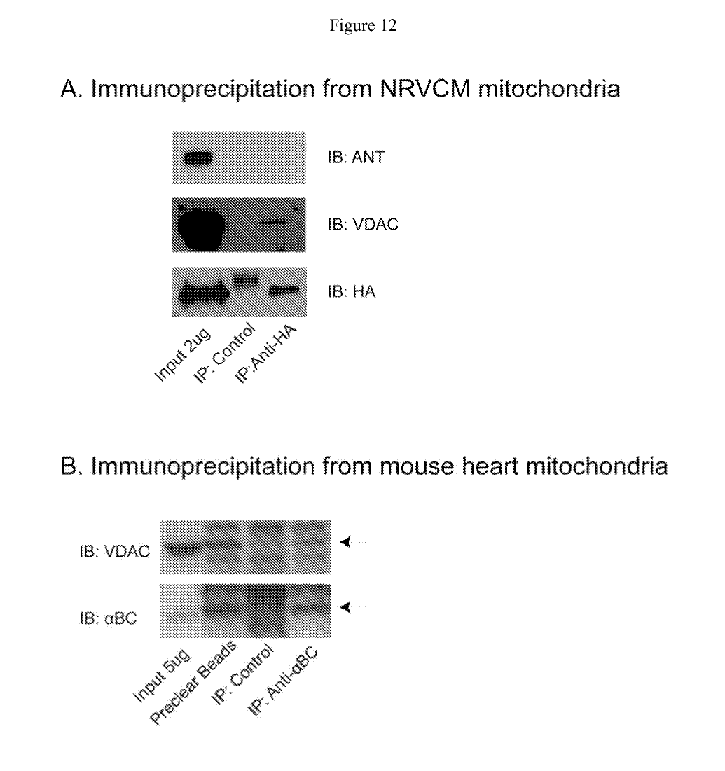

FIG. 12—Co-immunoprecipitation of αBC and VDAC from neonatal rat cardiomyocyte mitochondria and mouse heart mitochondria: Panel A—Immunoprecipitations were performed on mitochondrial extracts using either anti-HA antibody conjugated to agarose beads or control non-immune mouse antibody conjugated to agarose beads. Immunoprecipitations were then separated by SDS-PAGE and analyzed by western blotting for the presence of ANT, VDAC and αBC. Lanes containing control beads did not demonstrate any staining Lanes containing HA-beads stained positive for both αBC and VDAC. Panel B—Immunoprecipitations were also performed on mitochondrial extracts purified from mouse hearts that had been exposed to 30I/10R on a Langendorff apparatus. Anti-αBC antibody was used to precipitate endogenous αBC. The immunoprecipitations were separated by SDS-PAGE and analyzed for the presence of αBC and VDAC. The control lanes did not demonstrate any significant staining. However, anti-αBC lanes stained positive for both αBC and VDAC.

Since HA-αBC-AAE was associated with isolated mitochondria, immune-precipitation experiments were carried in attempts to identify putative mitochondrial αBC binding partners. Accordingly, HA-αBC was immunoprecipitated from mitochondria isolated from αBC-AAE-AdV-infected cultured cardiac myocytes. Immunoprecipitates were then analyzed by immunoblotting for several potential outer mitochondrial membrane binding partners thought to participate in cytochrome c release, adenine nucleotide translocase (ANT) or voltage-dependent anion channel (VDAC). While the blot for ANT did not reveal evidence of binding (FIG. 12A, IB: ANT lane 3), the blot for VDAC demonstrated that this protein bound to HA-αBC (FIG. 12A, IB: VDAC, lane 3), consistent with a role for VDAC in mediating the protective effects of αBC at mitochondria. To examine is this interaction occurs in vivo, αBC was immunoprecipitated from mitochondria isolated from mouse hearts that had been treated with 30I/10R prior to subcellular fractionation. Consistent with the cell culture findings, VDAC co-immunoprecipitated with αBC (FIG. 12B).

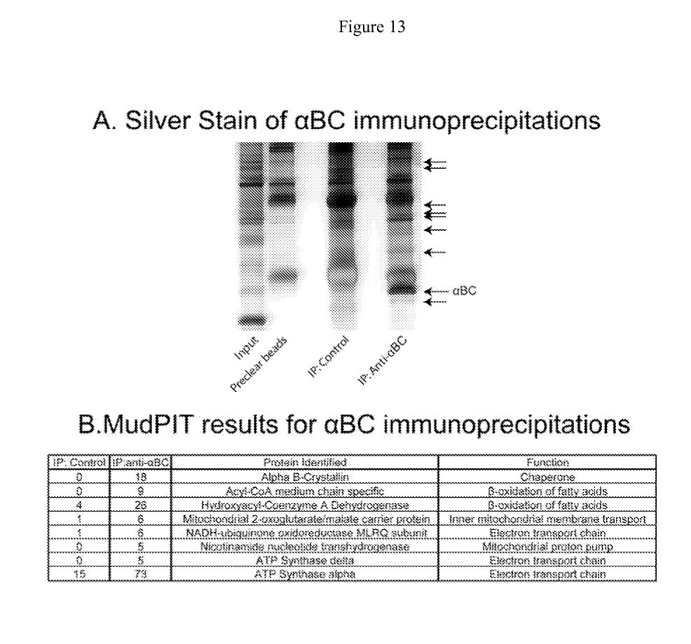

To identify other targets of αBC at the mitochondria, αBC was again immunoprecipitated from mouse heart mitochondria isolated following treatment with 30I/10R on the Langendorff apparatus. These samples were separated on an SDS-PAGE gel and silver stained. Silver staining revealed several unique bands resulting from αBC immunoprecipitation when compared to the control (FIG. 13A, Arrows). In order to identify, these targets αBC and control immunoprecipitations from mitochondrial fractions from hearts treated with either 30I/10R were submitted to MudPIT (Multidimensional Protein Identification) analysis. MudPIT analysis identified 8 mitochondrial proteins as potential targets of αBC during ischemia (FIG. 13B).

### 4. Discussion

The results of this study show that in the mouse heart, ischemia causes a striking quantitative disappearance of αBC from the cytosol (FIG. 8) that occurs on a timeframe coordinate with its appearance in other subcellular fractions, including mitochondria. Although the mechanism responsible for ischemia-mediated translocation of αBC to mitochondria is not known, previous studies on myocardial and other cell types suggest that αBC's propensity to bind to other proteins as a chaperone is likely to be at least partly responsible. For example, αBC has been shown to bind to numerous sarcomeric proteins, where it is believed to contribute to maintaining contractile element integrity during stress [168-172, 186, 190]. In human lens epithelial cells αBC has been shown to bind to the BH3-only proteins, Bax and Bcl-xₛ in the cytosol; since αBC remains in the cytosol in this cell type, this binding inhibits translocation of these proteins to mitochondria, thereby inhibiting apoptosis [177]. Perhaps the translocalization of αBC to mitochondria in cardiac myocytes may involve its binding to proteins like Bax and/or Bcl-xₛ, an interaction which, in contrast to epithelial cells, may not inhibit the migration of these Bcl family members to mitochondria. In this case, it may be possible that αBC modulates Bax and/or Bcl-xₛ pore-formation at the mitochondrial outer membrane (MOM), which would inhibit cytochrome c release and decrease mitochondrial-dependent apoptosis. It is also possible that the unfolding of MOM proteins attracts chaperones, like αBC.

For example, the MOM protein, TOM20, is prone to unfolding during myocardial ischemia, and other chaperones are known to bind to TOM20 under these conditions [191]. Additionally, it has been postulated that mitochondrial permeability transition pore (MPTP) proteins unfold during oxidative stress, and that modulating this unfolding through the binding of chaperones may also modulate permeability transition and the associated cell death [188].

FIG. 13—Proteomics analysis of interactions between αBC and components of the mitochondria: Panel A—Immunoprecipitations using anti-αBC and control antibodies were performed on extracts of mouse heart mitochondria that had been isolated following 30I/10R on a langendorff apparatus. Immunoprecipitations were separated by SDS-PAGE and silver-stained. Unique bands resulting from αBC immunoprecipitation are indicated by arrows. Panel B—Results of the MudPIT analysis revealed a number of proteins that were immunoprecipitated in a specific manner.

In the present study, the kinetics of ischemia-mediated αBC translocation and phosphorylation were consistent with the possibility that at least a portion of αBC phosphorylation occurred in the mitochondrial fraction. The known dependence of αBC phosphorylation on serine-59 by p38-mediated activation of MAP kinase activated protein kinase-2 (MK2) is consistent with the findings of the current study that during ischemia, activated p38 also translocated to mitochondria (FIG. 9C) [111]. Previous studies showing that both p38 and MAPKAP-K2 translocate to mitochondria during stress, and that αBC can form a complex with p38 and MAPKAP-K2, suggest that αBC might translocate to mitochondria as part of a p38 signalsome [192].

The nature of the physical association of αBC with mitochondrial fractions was also examined in the present study. It was shown that at least half of the phospho-αBC-S59 was susceptible to proteolytic degradation, indicating that at least a portion of it localizes to the surface of mitochondria during ischemia, while the remainder, being protease resistant, may be intramitochondrial (FIG. 10). The potential functional consequence of the association of αBC with mitochondria was examined in cultured cardiac myocytes, where it was shown that αBC-AAE, which mimics phospho-αBC-S59, decreased the oxidative-stress dependent release of cytochrome c (FIGS. 11A and 11B), consistent with our previous observation that αBC-AAE reduces mitochondria-dependent apoptosis. Finally, the co-immunoprecipitation of αBC-AAE and VDAC from NRVCM's (FIG. 12A) as well as the co-immunoprecipitation of endogenous αBC and VDAC support the hypothetical physical association between αBC-AAE and a key MOM protein known to regulate mitochondrial function during oxidative stress.

What role does VDAC play and how could the interaction of αBC with VDAC during ischemia modulate its role in a manner consistent with protection? VDAC was once thought to be a component of the MPTP, which opens during reperfusion leading to mitochondrial uncoupling, decreased ATP synthesis and eventual necrosis or apoptosis. However, more recent studies showing continued function of the MPTP in mitochondria that lack all 3 isoforms of VDAC convincingly demonstrated that VDAC is not an integral part of the MPTP [193]. Nonetheless, VDAC lies at the convergence of cell survival and death pathways, and has been described as a global regulator of mitochondrial function [187, 189]. VDAC plays an important role in ion and metabolite transport into and out of mitochondria, and its ability to close during ischemia may contribute to the global mitochondrial suppression thought to be key to survival during reperfusion [188]. VDAC is also thought to form channels through which cytochrome c can be released from stressed mitochondria. Moreover, the binding of various pro- and anti-apoptotic proteins to VDAC, such as tBID, Bax, and Bcl-X_{L}, regulate VDAC opening and, thus, VDAC-dependent cytochrome c release [187]. Accordingly, it is possible that αBC modulates mitochondrial cytochrome c release through either direct or indirect binding to VDAC. In fact, since αBC has already been shown to bind to Bax, which binds to VDAC, it is reasonable to hypothesize that αBC may exist in a complex with BAX and VDAC in a manner that regulates pore formation.

Furthermore, the preliminary MudPIT studies provide evidence that αBC may interact with certain metabolic proteins, including members of the electron transport chain (FIG. 13B). These findings are consistent with the results of the protease protection experiment (FIG. 10) that suggest a portion of the αBC found at the mitochondria exists within the MOM. Moreover, they are also consistent with mitochondrial respiration studies performed on isolated mitochondria from the hearts of αBC/HSPB2 double knockout mice that show reduced complex I activity [194]. These findings strongly suggest an entirely new role for αBC in regulating metabolism.

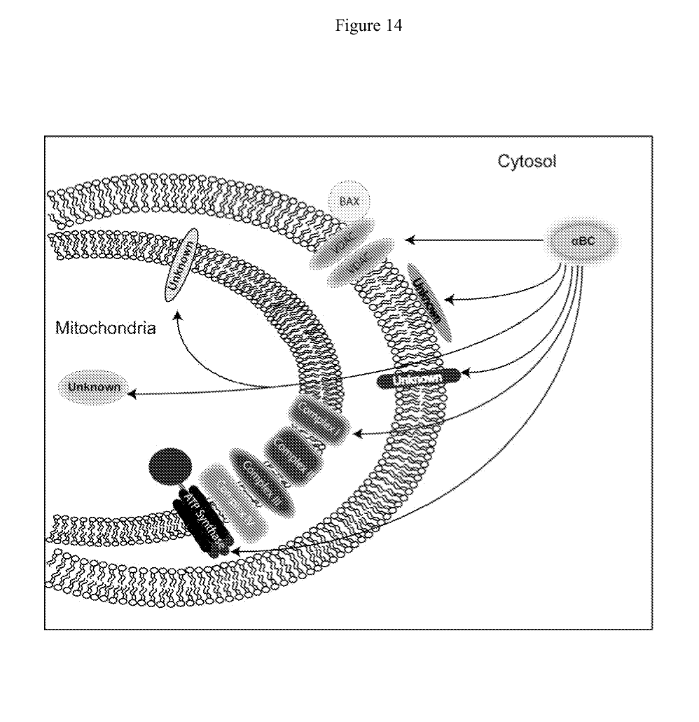

Until recently, the conventional wisdom regarding αBC within cardiomyocytes was that, in response to stresses, such as heat shock or I/R, αBC translocates out of the cytosol and moves to the sarcomeres. While, the sarcomeres are an important target of αBC during stress, this study and previous studies from our lab during the last several years have established that mitochondria are an equally important target [98, 108]. Consistent with our previous findings, we have demonstrated that the presence of αBC at the mitochondria is important for maintaining mitochondrial function and integrity. Furthermore, we have identified physical interactions between αBC and several key components of the mitochondria, however, the current list should in no way be considered all inclusive and undoubtedly many more interactions will be uncovered (FIG. 14).

FIG. 14—Summary of findings regarding αBC translocation to the mitochondria αBC was found to translocate to the mitochondria in response to I/R and prevent cytochrome c release. Further examination revealed a portion of αBC may exist within the MOM. Consistent with this finding, αBC was found to interact with a number of proteins that exist both outside and within the MOM.

Importantly, this study revealed that αBC translocates to the mitochondria and is phosphorylated within a time frame consistent with protecting mitochondrial function and integrity during I/R and prevents cytochrome c release. Given previously published studies, it appears that αBC can regulate early apoptotic signaling through interactions with pro-apoptotic Bcl family members, prevent cytochrome c release from mitochondria, and inhibit activation of effector caspases. Therefore, the findings presented bolster the role of αBC as a powerful, anti-apoptotic protein and suggest that αBC is a key regulator at all levels of mitochondrial dependent apoptosis signaling.

### Chapter Three

### 1. Introduction

Following an MI, blood flow must be restored to the ischemic region of the heart if the tissue is to survive. However, while reperfusion is a requirement for survival of the tissue, paradoxically, it also results in significant tissue damage [14]. This is due to the burst of ROS generation that follows reperfusion of the ischemic tissue. This generation of ROS can rapidly overwhelm cellular antioxidant systems, resulting in widespread cellular damage that can lead to cell death [31, 80]. Current clinical protocols for treating MI focus primarily on restoring blood flow to the ischemic tissue and provide no protection against ROS related reperfusion injury [195]. Therefore, antioxidant systems and the signaling pathways that control their activity have been a subject of intense interest amongst cardiovascular researchers.

Members of the sHSP family of proteins have been shown to enhance antioxidant activity and prevent oxidative stress associated cellular damage and cell death. The most well studied sHSP in this regard is HSP 25/27. Expression of HSP 25/27 has been shown to increase intracellular glutathione levels in several cell lines including L929 (fibrosarcoma cell line) and C2C12 (skeletal muscle satellite cell line) [196, 197]. This increase in glutathione was associated with reduced protein oxidation, lipid peroxidation and cell death when cells were challenged with either H₂O₂ or tumor necrosis factor alpha (TNFα). Examination of the mechanism by which HSP25/27 elicits this effect has shown that HSP25/27 expression can increase G6PD activity, which enhances glutathione recycling, and helps to preserve intracellular levels glutathione.

Several studies examining the protective effects of αBC against I/R damage have provided results suggesting that, like HSP25/27, αBC may also influence antioxidant systems. The HSP25/27 studies referenced above were some of the earliest studies to suggest that αBC could provide protection against oxidative stress. In parallel experiments the same group created a stably transfected line of L929 cells that expressed αBC. These cells were more resistant to ROS related damage and demonstrated lower cell death when they were challenged with TNFα. Using the same stably transfected cell line, the same group also demonstrated that αBC could increase intracellular glutathione and protect against H₂O₂-induced cell death.

Studies utilizing transgenic and knockout mice have suggested that αBC plays a role in protecting the heart from oxidative stress. Hearts from mice with a cardiac-specific overexpression of αBC were shown to have greater functional recovery and reduced cell death following ex vivo I/R[160]. Consistent with this was the finding that following I/R, the αBC over expressing hearts were found to contain lower levels of malondialdehyde, a product of lipid peroxidation, compared to hearts from control animals. Conversely, the hearts of HSF1 knockout mice were found to express 40% less αBC and 72% less HSP25, compared to control animals [121]. The hearts from these animals were found to have a significantly lower reduced glutathione to oxidized glutathione ratio and increased levels of protein carbonyls when compared to control animals, even in the absence of any stressors. Taken together these results suggest that αBC may protect hearts from oxidative stress by influencing the glutathione system.

There have been no reports that explored the mechanism(s) by which αBC influences the glutathione system. Furthermore, there have been no published studies that examined the effects of αBC expression on glutathione levels or the activity of glutathione related enzymes in the heart. Therefore, this study was undertaken to examine the effects of αBC expression on glutathione levels in the heart and to determine the mechanism(s) by which αBC may provide protection against oxidative stress.

### 2. Materials and Methods

A. Animals—Approximately 40, 10-20 week-old 129SVEV_{TAC} and αBC/HSPB2 double knockout mice (Mus musculus) were used in this study. All procedures involving animals were performed in accordance with institutional guidelines. The animal protocol used in this study was reviewed and approved by the San Diego State University Institutional Animal Care and Use Committee.

### B. Measurement of Reduced and Oxidized Glutathione

Measurement of Reduced Glutathione and Glutathione disulfide were performed as previously described with modifications for use in a 96 well plate format [30].

C. Measurement of glucose-6-phosphate Dehydrogenase Activity

Glucose-6-Phosphate Dehydrogenase activity was measured as described previously [198] with modifications for use in a 96 well plate format. Briefly, 10 ug of sample was added to reaction buffer 1 (86.3 uM Triethanolamine buffer pH 7.6, 6.7 μM MgCl₂, 100 μM NADP, 200 μM Glucose-6-Phosphate, 200 μM 6-Phosphgluconate) and to reaction buffer 2 (86.3 uM Triethanolamine buffer pH 7.6, 6.7 uM MgCl₂, 100 uM NADP, 200 uM 6-Phosphgluconate) to start the reactions which were followed at 340 nm for 5 min at 22° C. (VERSAMAX™ microplate reader, Molecular Devices). Glucose-6-phosphate activity was determined by subtracting the activity of reaction 2 from reaction 1. nMoles NADPH were calculated using a molar extinction coefficient of 6250 M⁻¹ cm⁻¹.

### D. Measurement of Glutathione Reductase Activity

Glutathione Reductase activity was measured as previously described with modifications for use in a 96 well plate format[199]. Briefly, 50-100 ug of sample was added to reaction buffer (0.1M potassium phosphate buffer pH 7.4, 0.6 mM EDTA, 2 mM GSSG) to a total volume of 237.5 ul and allowed to incubate at 22° C. for 10 min. The reaction was started by the addition of 12.5 μl of 2 mM NADPH and was followed at 340 nm for 3 min. nMoles of NADPH were calculated using a molar extinction coefficient of 6250 M⁻¹cm⁻¹.

### E. Measurement of Caspase 3 Activity

Following transfection HeLa cells were plated at a density of 0.5×10⁶ cells per well in 6 well plates and allowed to recover overnight. The cells were then treated with 200 μM Hydrogen Peroxide in minimal media for 8 hours. Following the incubation cells were washed gently with ice cold 1×PBS and scraped in caspase assay buffer (50 mM HEPES pH 7.4, 0.1% CHAPS, 0.1 mM EDTA), sonicated briefly, and centrifuged at 15,000 g for 10 minutes at 4° C. 50 ul of sample per well was loaded into a black 96 well plate and combined with 10 ul of assay buffer and 45 ul of reaction buffer (50 mM HEPES, 0.1% CHAPS, 0.1 mM EDTA, 0.01 mM DTT, 40 μM N-Acetyl-Asp-Glu-Val-Asp-7-Amido-4-trifluomethyl-coumarin). The reaction was incubated in the dark at 37° C. for 1 hour. Following the incubation period the plate was read on a 96 well fluorimeter with the excitation and emission wavelengths set to 400 nm and 505 nm, respectively. Sample background fluorescence was determined using a separate reaction where the caspase 3 substrate in the reaction buffer was replaced with dimethyl sulfoxide.

### F. Co-Immunoprecipitation of αBC and Glutathione Reductase

HeLa cells were transfected with a plasmid encoding wildtype αBC with an n-terminal HA tag and allowed to recover on a 150 mm dish for 24 hours. HeLa cells were then scraped in RIPA buffer, briefly sonicated, and centrifuged at 15,000 g for 10 minutes. For each immunoprecipitation 30 ug of HeLa cell extract was incubated overnight at 4° C. in a total volume of 500 ul with either HA antibody or control mouse IgG antibody conjugated to agarose beads. Following incubation the beads were washed twice with PBS-T (1×PBS with 0.05% TWEEN 20™) followed by 3 washes with 1×PBS. Antibody-protein complexes were released from the agarose beads by boiling samples in Laemelli buffer. Samples were then separated by SDS-PAGE and analyzed by western blotting.

### G. Statistics

Data were analyzed by students t-test or ANOVA followed by nueman-keuls post hoc where appropriate. Data are presented as means+/−standard error of the mean (SEM).

### 3. Results

Alpha-BC knockout mice were created by Dr. Eric Wawrousek's lab by deleting part of the promoter region and a portion of exon 1 of the αBC gene [162]. However, this strategy created mice that also harbor a knockout of the neighboring gene, HSPB2. These mice (αBC/HSPB2-KO) were previously demonstrated to be more susceptible to I/R damage and were used in this study to examine the effect of αBC expression on glutathione levels in the heart. To analyze glutathione levels, hearts were quickly excised from anesthetized animals, weighed and then homogenized in 5% sulfosalycilic acid. Glutathione levels were assessed using a glutathione reductase cycling assay. Total glutathione content, in hearts from αBC/HSPB2-KO mice, was found to be reduced 43% compared to hearts from control animals (FIG. 1A). Conversely, the oxidized glutathione content of αBC/HSPB2-KO hearts was found to be 54% greater than that of control hearts (FIG. 1B). This in turn resulted in a decrease in the reduced glutathione to oxidized glutathione ratio from 56.79±2.82 for hearts from control animals to 21.73±0.818 for αBC/HSPB2-KO hearts, a reduction of more than 60% (FIG. 1C).

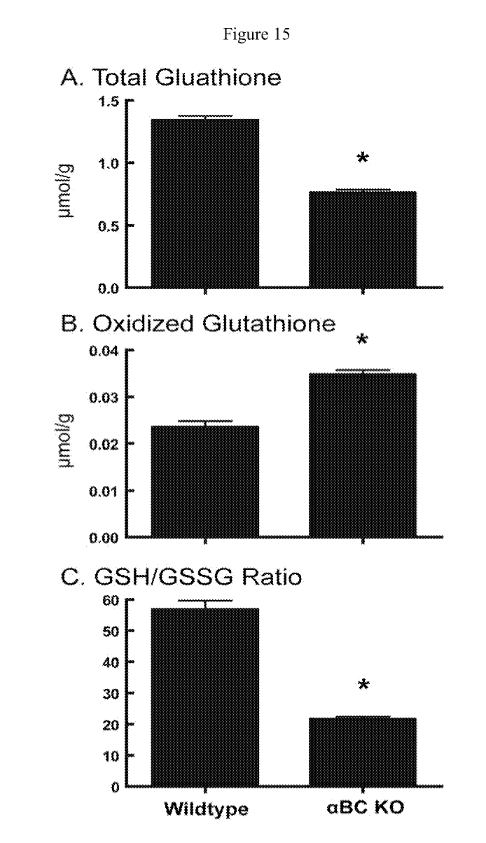

FIG. 15—Measurement of total glutathione, oxidized glutathione, and reduced glutathione to oxidized glutathione ratios in hearts from αBC/HSPB2 knockout mice. see: Morrison L E, Whittaker R J, Klepper R E, Wawrousek E F, Glembotski C C. Roles for alphaB-crystallin and HSPB2 in protecting the myocardium from ischemia-reperfusion-induced damage in a KO mouse model. Am J Physiol Heart Circ Physiol. 2004 March; 286(3):H847-55. Panel A: Total glutathione was measured in hearts from wildtype and αBC/HSPB2 KO mice using a glutathione reductase cycling assay (n=3 for each group). Panel B: Oxidized glutathione was measured in the same samples following derivativation of reduced glutathione with 2-vinylpyridine. (n=3 for each group). Panel C: Ratio of reduced glutathione to oxidized glutathione; reduced glutathione was calculated from total and oxidized glutathione measurements.

Following analysis of the glutathione levels in hearts of αBC/HSPB2-KO the activities of the enzymes associated with glutathione cycling were measured in whole heart extracts. Measurement of G6PD activity revealed no significant difference in activity between hearts from αBC/HSPB2-KO mice and control animals (FIG. 2A). However, measurement of GR activity revealed a 23% reduction in activity in hearts from αBC/HSPB2-KO mice when compared to control animals (FIG. 2B). Deletion of the αBC and HSPB2 genes had no effect on protein expression levels of either G6PD and GR (FIG. 2C).

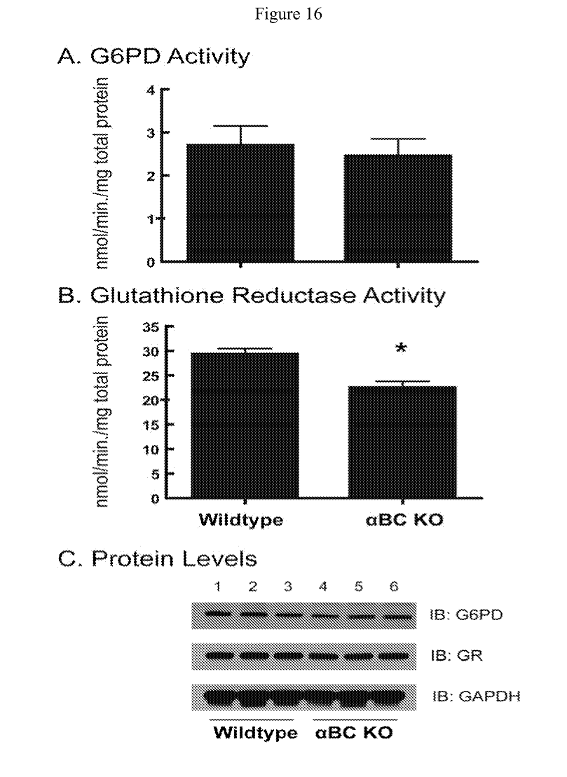

FIG. 16—G6PD, GR activity and protein levels in αBC/HSPB2 knockout mouse hearts. Panel A: G6PD activity was measured in hearts from wildtype and αBC/HSPB2 KO mice (n=3 for each group). Panel B: GR activity was measured in hearts from wildtype and αBC/HSPB2 KO mice (n=3 for each group). Panel C: Protein levels of both G6PD and GR were analyzed by western blotting in hearts from wildtype (lanes 1-3) and αBC/HSPB2 KO (lanes 4-6) hearts.

HeLa cells were chosen as a model system to further examine the effects of αBC expression on glutathione levels and to explore how αBC interacts with glutathione cycling enzymes. HeLa cells are an excellent complement to the αBC-KO hearts as they express neither αBC nor HSPB2 (FIGS. 3A and 3B). Importantly, transfection of HeLa cells with a plasmid that expresses αBC did not change the levels of the related sHSP, HSP27, which is known to influence glutathione levels and glutathione cycling enzyme activity (FIG. 3B).

Glutathione levels in HeLa cells were measured using the same glutathione reductase cycling assay that was used to assess glutathione levels in hearts. Under basal conditions, αBC expression had no effect on total glutathione levels (FIG. 4A). Oxidized glutathione levels were slightly lower in cells expressing αBC, nevertheless, this difference did not reach statistical significance (FIG. 4B). However, the expression of αBC did result in a 48% higher ratio of reduced glutathione to oxidized glutathione (FIG. 4C).

Following a 90 minute treatment with H₂O₂, total glutathione levels were found to be significantly higher in HeLa cells transfected with αBC when compared to cells transfected with control plasmid (FIG. 5A). Conversely, oxidized glutathione levels were found to be significantly reduced in HeLa cells transfected with αBC (FIG. 5B). This resulted in a 230% higher reduced glutathione to oxidized glutathione ratio in cells transfected with αBC compared to cells transfected with control plasmid (FIG. 5C).

The effect of αBC expression on the activity of glutathione cycling enzymes was assessed in the HeLa cells. Expression of αBC had no effect on the activity of G6PD in HeLa cells (FIG. 6A). However, transfecting HeLa cells with αBC resulted in a 63% increase in GR activity when compared to cells transfected with control plasmid (FIG. 6B). Protein levels of G6PD and GR were assessed by western blot analysis. Expression of αBC had no effect on protein levels of either G6PD or GR (FIG. 6C).

To determine if the enhanced glutathione levels had made the cells more resistant to oxidative stress, caspase 3 activity was analyzed in cells transfected with or without αBC and treated with H₂O₂. Consistent enhanced glutathione levels providing protection against oxidative stress, caspase 3 activity was found to be reduced following treatment with H₂O₂, in cells transfected with αBC when compared to cells transfected with control plasmid (FIG. 7).

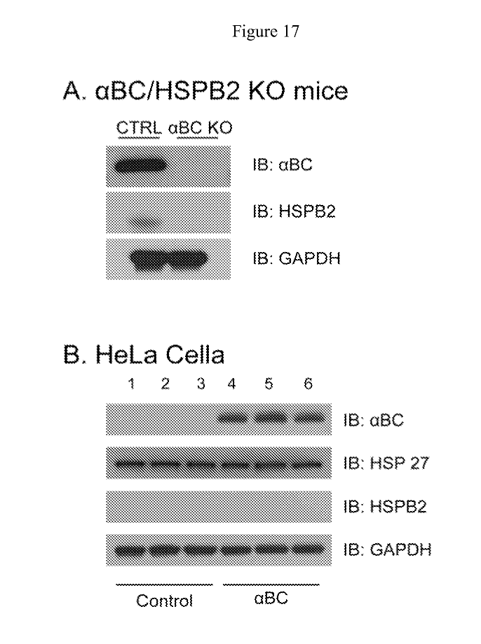

FIG. 17—Comparison of protein expression in αBC/HSPB2 KO hearts and HeLa cells. Panel A: Western blots to examine expression of αBC and HSPB2 in hearts from wildtype and αBC/HSPB2 KO mice. Panel B: Western blots to examine expression of αBC, HSP27, and HSPB2 in HeLa cells transfected with either a control plasmid (lanes 1-3) or a plasmid which expresses αBC (lanes 4-6).

To determine if αBC interacted either directly or as part of a complex that included GR, immunoprecipitations were performed using cell extracts from HeLa cells that had been transfected with αBC plasmid. Immunoprecipitations were performed using either agarose beads with non-immune mouse antibody, as a control, or agarose beads with an anti-HA antibody. Control immunoprecipitations were negative for both αBC and GR by western blot analysis. However, using anti-HA beads resulted in co-immunoprecipitation of αBC and GR (FIG. 8A). To better understand the nature of the interaction between αBC and GR, the enzyme activity of purified GR was analyzed in vitro in the presence and absence of αBC. Compared to a BSA control, αBC increased GR activity by a statistically significant 18% (FIG. 8B).

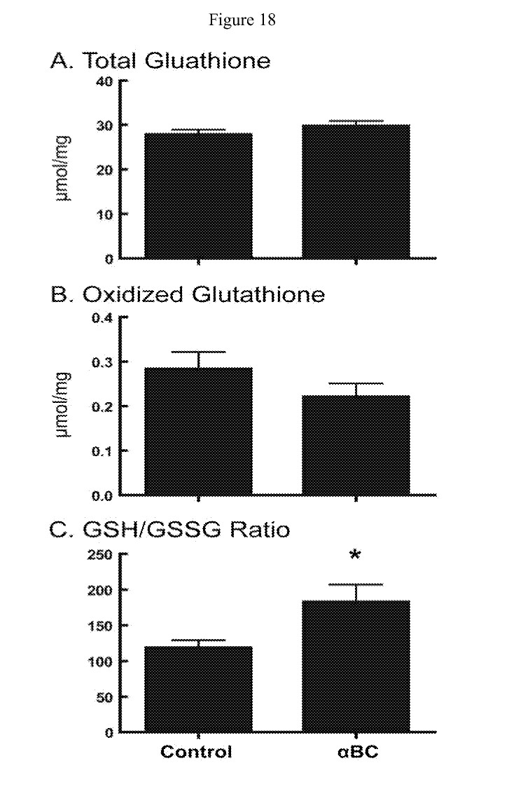

FIG. 18—Measurement of total glutathione, oxidized glutathione and reduced glutathione to oxidized glutathione ratios in HeLa cells transfected with αBC plasmid. Panel A: Total glutathione was measured in HeLa cells transfected with either control plasmid or and plasmid which expresses αBC using a glutathione reductase cycling assay (n=27 for each group). Panel B: Oxidized glutathione was measured in the same samples following derivitization of reduced glutathione with 2-vinylpyridine. (n=27 for each group) Panel C: Ratio of reduced glutathione to oxidized glutathione; reduced glutathione was calculated from total and oxidized glutathione measurements.

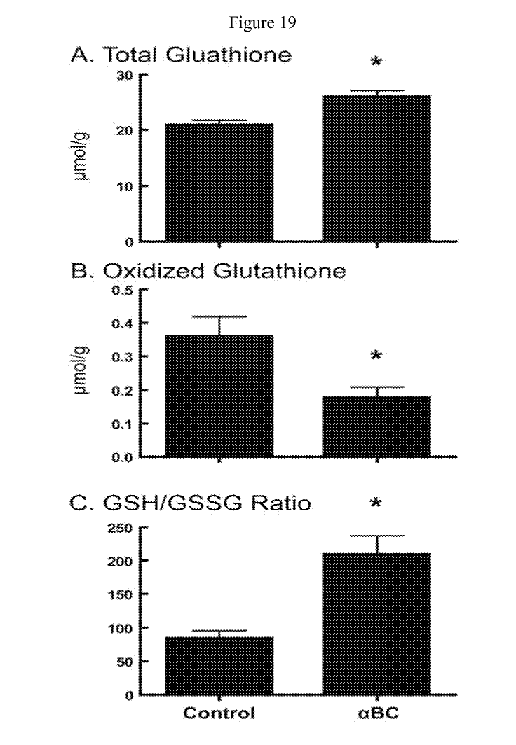

FIG. 19—Measurement of total glutathione, oxidized glutathione and reduced glutathione to oxidized glutathione ratios in HeLa cells transfected with αBC plasmid following 90 min treatment with 200 μM H₂O₂. Panel A: Total glutathione was measured in HeLa cells transfected with either control plasmid or and plasmid which expresses αBC using a glutathione reductase cycling assay (n=27 for each group). Panel B: Oxidized glutathione was measured in the same samples following derivitization of reduced glutathione with 2-vinylpyridine. (n=27 for each group). Panel C: Ratio of reduced glutathione to oxidized glutathione; reduced glutathione was calculated from total and oxidized glutathione measurements.

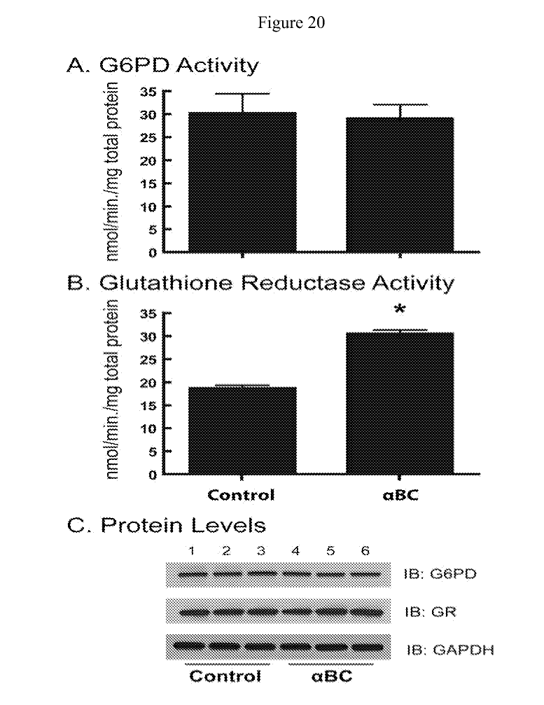

FIG. 20—G6PD, GR activity and protein levels in αBC/HSPB2 knockout mouse hearts. Panel A: G6PD activity was measured in HeLa cells transfected with either control plasmid (n=15) or a plasmid which expresses αBC (n=18). Panel B: GR activity was measured in HeLa cells transfected with either control plasmid or a plasmid which expresses αBC (n=13 for each sample). Panel C: Protein levels of both G6PD and GR were analyzed by western blotting HeLa cells transfected with either control plasmid (lanes 1-3) or a plasmid which expresses αBC (lanes 4-6).

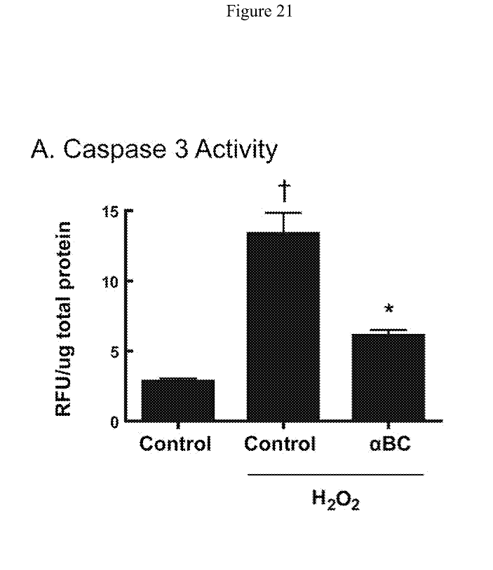

FIG. 21—Protection against hydrogen peroxide induced activation of apoptosis by αBC expression. Caspase 3 activity was measured using caspase 3 substrate that fluoresces upon cleavage of the target sequence. Activation of caspase 3 was measured in cell extracts from cells transfected with either control plasmid or αBC plasmid treated+/−200 μM H₂O₂ for 8 hours in minimal media.

### 4. Discussion

Cardiomyocytes require large amounts of ATP in order to contract and contribute to the pumping action of the heart. In order to meet this energy requirement mitochondria make up nearly 40% of the volume of the cardiomyocyte [200]. Even under basal conditions, approximately 2-5% of the oxygen metabolized by cardiomyocyte mitochondria result in ROS generation [14]. However, during the first few minutes of reperfusion following an extended period of ischemia, ROS generation increases nearly 7.5-fold over basal levels [20]. This rapid increase in ROS generation results in the cardiomyocyte entering oxidative stress, a condition where the cell's antioxidant systems are no longer able to detoxify ROS species at the same rate as they are being generated.

During oxidative stress, the cell is extremely susceptible to damage as ROS can attack nearly every component of the cell [17]. Understanding how antioxidant systems are controlled will help lead to antioxidant therapies to be dispensed as adjuvants to the reperfusion therapies currently administered to patients diagnosed with MI. In the present study, we found that αBC gene abalation reduced glutathione recycling in the mouse heart, resulting in lower levels of total glutathione and a lower reduced glutathione to oxidized glutathione ratio. This most likely arises from the significant decrease in GR activity seen in the knockout mice. Taken together, these results suggest that loss of αBC leaves the heart with a reduced ability to detoxify ROS. This is consistent with our previous report that demonstrated that loss of αBC renders hearts more susceptible to I/R damage [161].

However, our mouse model is not perfect, the strain of mice that we studied also harbor a deletion of the related HSPB2 gene. Even though HSPB2 is expressed at much lower levels than αBC, it is formally possible that it contributes to enhancing glutathione recycling. Fortunately, HeLa cells provide an easily manipulated cell culture model that mimics the αBC and HSPB2 deletions of the knockout mice. Transfection of HeLa cells with a plasmid which expresses αBC results in an increase in the reduced glutathione to oxidized glutathione ratio. When the transfected cells were treated with H₂O₂ the cells transfected with αBC demonstrated significantly higher levels of total glutathione compared to control transfected cells. Following H₂O₂ treatment, levels of oxidized glutathione were significantly reduced in cells transfected with αBC and contributed to a much higher reduced glutathione to oxidized glutathione ratio in these cell. These changes are associated with an increase in GR activity observed in cells transfected with αBC.

Generally a higher reduced to oxidized glutathione ratio suggests a cell is better able to detoxify ROS and prevent injury. Consistent with this theory, cells transfected with αBC experienced less and apoptosis following H₂O₂ treatment compared to cells transfected with control plasmid.

The question remains however, why expression of αBC did not increase glutathione levels under basal conditions the same way it did in the heart. More than likely this is due to several differences between the models. The first arises due to the cell physiology, HeLa cells are not packed with mitochondria the way that cardiomyocytes are, therefore the endogenous ROS production of HeLa cells is presumably much lower and places a lower demand on the antioxidant systems. The second reason stems from differences between studying cell culture in the dish and tissues. Cells in the culture dish experience a much higher oxygen tension than cells within the organ, therefore they may be pre-adapted to growing in an environment that would stimulate more ROS generation.

Nonetheless, the results demonstrate that αBC can protect cells from ROS damage by increasing glutathione recycling. To understand the mechanism behind this protection we explored the interaction of GR and αBC. Immunoprecipitation experiments confirm that these two proteins interact within the cell. However, co-immunoprecipitation tells us nothing about the nature of the interaction, specifically whether the two proteins interact directly or as part of a complex. To answer this question we explored the effects of including αBC in in vitro activity assays using GR. Compared to the BSA control, αBC increased GR activity 18%. While at first blush that might appear to be a rather modest increase, it is consistent with the 23% decrease in GR activity seen in αBC/HSPB2 KO hearts. The increase is also noteworthy considering all substrates where provided at saturating levels.

The nature of the interaction between the two proteins and how that results in increased GR activity remains to be determined. More than likely, the chaperone activity of αBC is somehow involved. Future studies using αBC mutants which lack or have reduced chaperone activity should provide insight in this regard. A requirement for the presence of a chaperone might suggest that GR is more active in an unstable conformation and that αBC helps it to maintain this conformation. Unfortunately, such hypotheses can't be explored readily without performing X-ray crystallographic studies. However, what is clearly apparent from this study is that enhancing glutathione recycling is yet another way that αBC can protect the heart from I/R.

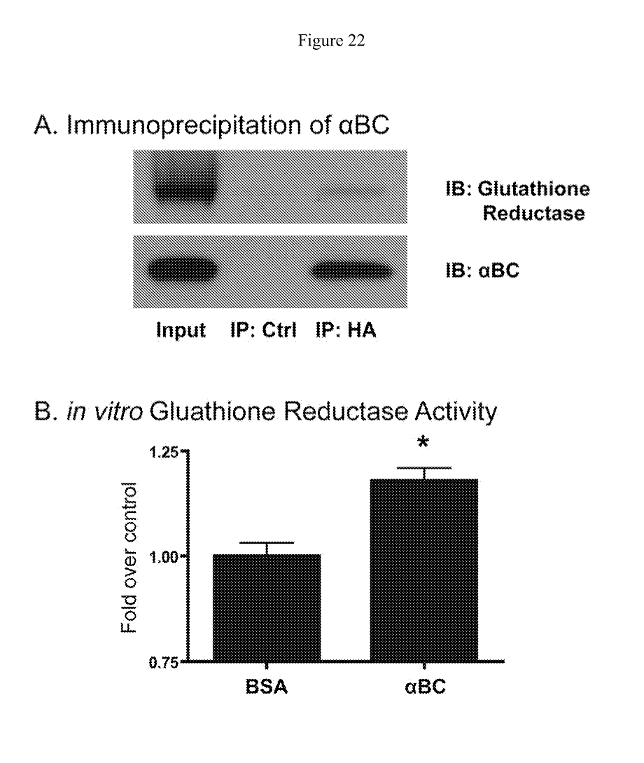

FIG. 22—Co-immunoprecipitation of αBC and GR and enhancement of GR activity in vitro by purified αBC. Panel A: Immunoprecipitation from whole cell extracts of HeLa cells transfected with αBC plasmid using either control or anti-HA antibodies, followed by western blotting for αBC and GR. Panel B: in vitro GR activity assays using purified GR and purified αBC, BSA is used as a control protein (n=12 for each group). Proteins were added at a 1:4 molar ratio to GR.

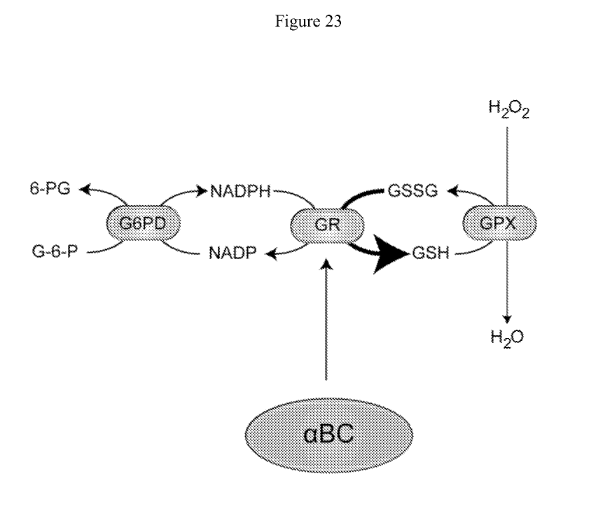

FIG. 23—Summary of findings regarding the effects of αBC expression on gluthathione recycling.

The results of this study demonstrate αBC, and the compositions of the invention, can protect cells from oxidative stress by interacting with and increasing the activity of GR.

### Chapter Four

### 1. Introduction

Cell penetrating peptides (CPP's) are a relatively new tool that may provide the answer to one of the most formidable obstacles in research and medicine, introducing exogenous molecules into the cell [201]. The plasma membrane is perhaps the most complex organelle of the cell, but it's most basic functions, acting as a barrier and regulating import and export of molecules to and from the cell, provide a significant challenge to transferring what is learned in the lab to the clinical setting. The manipulations we make in the lab, such as introducing DNA or siRNA, cannot be easily or safely transferred to clinical practice. However, with the discovery of CPP's it may now be possible to not only transfer DNA and siRNA, but also proteins and small molecules across the plasma membrane and into cells [202].

Naturally occurring cell penetrating domains have been discovered in a number of proteins from a diverse array of organisms including, Drosophila, HIV-1 virus and yeast [203]. These cell penetrating domains have been shown to effectively cross cell membranes by themselves, and with attached cargo such as plasmids, proteins and small molecules. The term CPP encompasses both naturally occurring cell penetrating domains and synthetic CPP's, such as arginine polymers [204] [201]. CPP's are generally smaller than 30 amino acids and are rich in cationic residues. Arginine appears to be the favored residue; the most efficient CPP's are generally rich in arginine residues. Interestingly, outside of a high proportion of arginine residues, there is little sequence or structural similarity between the various CPP's.

CPP mediated delivery of proteins has proven to be an effective way to deliver proteins into cells in a manner that preserves their biological activity. CPP's have been used effectively to deliver protective proteins, such as HSP27 and Apoptosis repressor of caspase

(ARC), to cardiomyocytes in culture and in ex vivo perfused heart models [205, 206]. Even more promising are studies that demonstrate CPP's can be used to deliver proteins to the hearts of mice in vivo [207].

CPP delivery of proteins potentially has several advantages over mouse models generated by genetic manipulations. Transgenesis to generate over-expression models can result in undesirable transgene insertions, potentially creating genetic deletions that go undetected. Secondly, CPP delivery allows the researcher to precisely control the timing and the levels of the protein being introduced. Lastly, since it is unnecessary to breed out transgenic lines and maintain colonies, CPP delivery of proteins offer significant cost savings over traditional transgenic mouse models.

Importantly, beyond their use in the research laboratory, there is a very real potential for CPP's to become a vehicle for the delivery of therapeutics. The sort of manipulations we can make in the laboratory, such as maintaining mitochondrial integrity during stress by expression of αBC phospho-mimetics, are not possible in the clinic. Furthermore, even if the sorts of genetic manipulations we perform in the lab were feasible in human subjects, long term, chronic over-expression of many proteins that are believed to protect the myocardium during I/R may not be desirable. Therefore CPP's, if they can live up to their expectations, may prove to be powerful tools for the clinician.

For our work, we are interested in studying the effects of phosphorylation of αBC in protection of the myocardium during I/R. We decided that using CPP's to deliver different phospho-mimetics of αBC might be a viable option that would allow us to manipulate not only the levels of the phospho-mimetics in the myocardium but also the timing of their presence in relation to the I/R progression. Therefore, in this study, we examine the use of CPP's to uncover the role of phosphorylation of αBC in protecting the myocardium from I/R related injury and whether this a viable approach to utilizing the protective effects of αBC in the clinical setting.

### 2. Materials and Methods

A. Creation of tat-αBC expression constructs—Our lab had previously created mammalian αBC expression constructs for wildtype αBC and the phospho-mimetics, αBC-AAE and αBC-AAA in PCDNA3.1-. The bacterial expression construct containing the Tat domain and a 6× histidine tag for purification was a generous gift from Dr. Steve Dowdy's lab. Wildtype αBC and the phospho-mimetics were excised from the PCDNA3.1 expression vectors by restriction digest with XhoI and were ligated into the Tat-expression construct and transformed into XL1-Blue cells. Clones were selected on agar plates with ampicillin. Clones with the αBC gene inserted in the right orientation were selected following sequencing and grown up to isolate plasmid DNA. BL-21 cells were transformed with the Tat-αBC constructs and utilized for expression and purification of the Tat-αBC proteins. Glycerol stocks of Tat-GFP and Tat-beta galactosidase to be used as control proteins were a generous gift from Dr. Roberta Gottleib, San Diego State University, San Diego, Calif.

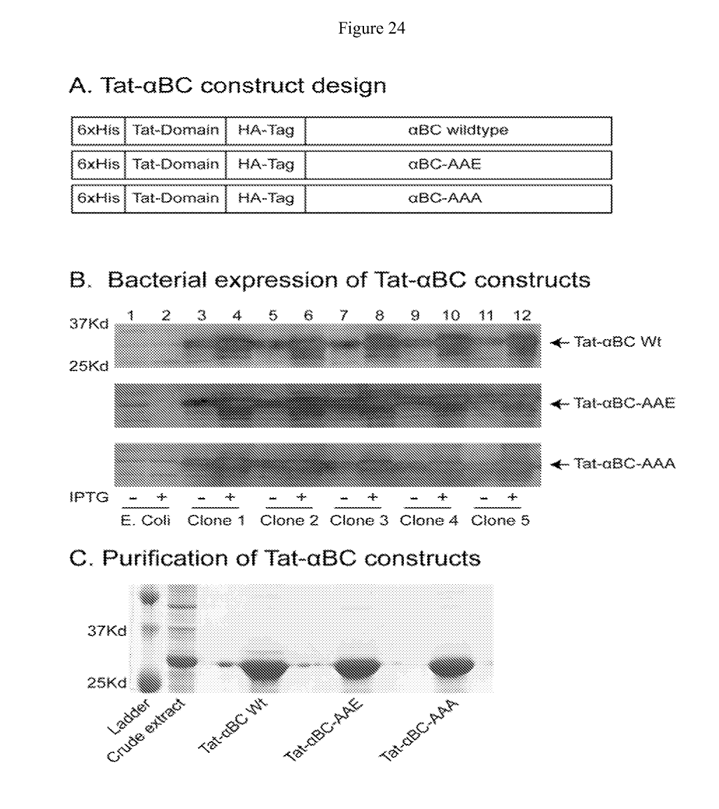

FIG. 24—Cloning, expression and purification of tat-αBC proteins:

Panel A: The tat-αBC proteins are fusion proteins that contain a 6× histidine tag for purification, the tat protein transduction domain, and an HA tag for detection. Panel B: Several clones that expressed the tat-αBC proteins in an IPTG inducible manner were isolated. Panel C: Purification on nickel-agarose columns resulted in isolated tat-αBC protein that was essentially free of contaminating protein.
B. Purification of tat-αBC proteins—Tat-αBC proteins were purified as previously described [206]. Briefly 3 ml cultures were grown overnight @ 37° C. with vigorous shaking in Luria broth (LB) with ampicillin. The following morning the 3 ml cultures were added to 1 L of fresh LB with ampicillin and allowed to incubate @ 37° C. until they became cloudy, at this point, IPTG was added to a final concentration of 500 μM. Incubation @ 37° C. continued for 6 hours at which point the cultures were spun down, washed twice with 1×PBS and the pellets were frozen overnight. The next day the pellets were thawed and suspended in 10 mls buffer Z (8M urea, 100 mM NaCl, 20 mM HEPES pH 8.0). Cells were sonicated with 3, 20 second pulses with a minimum of 30 seconds on ice between pulses. The cells were then centrifuged at 20,000 g for 20 min to pellet unbroken cells and cellular debris, the supernatant was collected. A 3 ml Ni-NTA column is prepared and equilibrated with 50 mls of buffer Z containing 10 mM imidazole. The supernatant is applied to the column and allowed to flow through by gravity. The column is then washed with 50 mls of buffer Z with 10 mm imidazole. The protein is eluted from the column with 10 mls buffer Z containing 250 mM imidazole. The protein is refolded using PD-10 desalting columns per the manufacturer's directions. Purified protein is then concentrated using a Millipore Centricon 70.
C. Treatment of NRVCM's with tat-αBC proteins—NRVCM's were isolated as described in chapter one and cultured in DMEM/F12 with 10% FBS for 2 days following isolation. On the third day Tat-αBC proteins were added to the media in various concentrations. After one hour the media was replaced with fresh media.
D. Confocal microscopy—Tat-αBC was conjugated to Texas-red fluorophore and re-purified on PD-10 desalting columns to exclude unconjugated label prior to being applied to NRVCM's. Following a 1 hour treatment with 200 μM Tat-αBC labeled with texas-red cells were washed twice with 1×PBS and fixed in 4% formaldehyde in PBS. The cells were then stained for α-Actinin using a FITC secondary and stained with TOPRO to identify nuclei. Images were captured on a Leica confocal microscope and associated software.
E. Heat shock Assay—Analysis of αBC translocation upon heat shock was performed as previously described. Briefly, following incubation with purified tat-αBC proteins, NRVCM's were placed at 44° C. 30 minutes. Cells were then trypsinized, collected and washed twice with 1×PBS. Cell pellets were resuspended in lysis buffer (10 mM Tris pH 7.5, 10 mM NaCl, 5 mM MgCl₂, 0.5% Triton x-100, 1 mM PMSF) and then centrifuged at 300 g for 5 minutes. Supernatant was collected and the pellet is resuspended in 4× laemelli buffer. Equal proportions of each sample were separated by SDS-PAGE and analyzed by western blotting.
F. ADH Aggregation assay—Were performed as previously described with modifications for use in a 96 well plate format [208].
G. Creation of αBC constructs for R9-C conjugation—αBC-AAE had been previously cloned into the PrsetA vector in order to purify it from E. coli. The αBC containing Prset plasmid was purified from an existing glycerol stock. The mutation to allow conjugation of the purified αBC-AAE to the R9-C peptides was added by utilizing mutagenesis to replace the tryptophan 6 amino acids prior to the start of the αBC-AAE gene with a cysteine using the primers: 5′ gataaggatcgatgcggatccgagctcg 3′ and 5′ cgagctcggatccgcatcgatccttatc 3′. The mutants were transformed into BL-21 cells and purified in the same manner described above.
H. R9-C peptides—R9-C peptides were acquired at >95% purity from Sigma Genosys.
I. Linkage of R9-C and αBC-AAE—Linkage of the modified αBC-AAE protein and the R9-C peptide were accomplished by incubation the peptide and the protein in equimolar concentration in 50 mM Tris pH 7.0 for 1 hour at room temperature.

### 3. Results

Wildtype αBC, αBC-AAE and αBC-AAA were excised from previously created mammalian expression constructs and ligated into the ptat-expression construct, a generous gift from Dr. Steve Dowdy. These constructs produce protein products with a 6× histidine tag, a Tat protein transduction domain, an HA-tag and either wildtype or one of the mutant forms of αBC (FIG. 24A). BL-21 cells were transformed with the Tat-αBC constructs. Clones were selected and analyzed for expression levels and IPTG inducibility (FIG. 24B). All five clones of each tat-αBC construct were found to express protein at similar levels. One clone for each construct was selected and used throughout the remainder of the study. The tat-αBC proteins were purified using a nickel agarose column. Each of the tat-αBC protein preparations were shown to be nearly free of contaminating proteins by Coomassie staining of SDS-PAGE gels (FIG. 24C lanes 4,6,8).

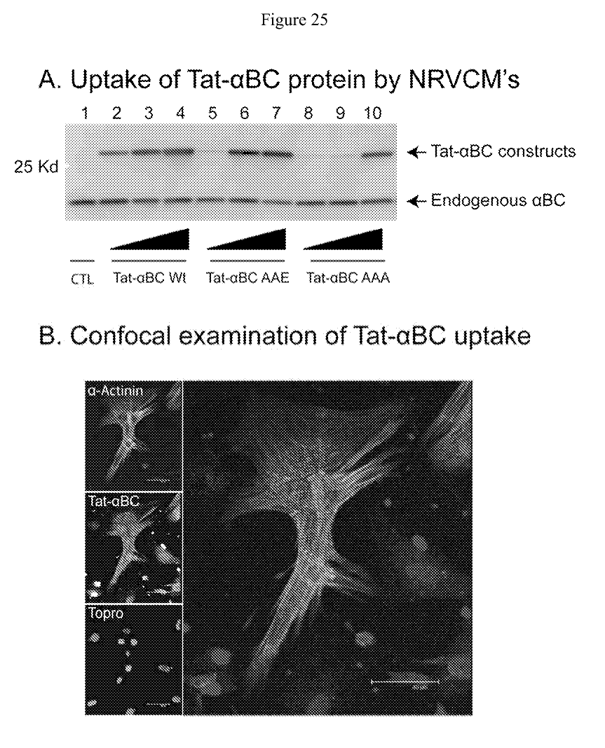

Following purification, uptake of the Tat-αBC proteins was tested in NRVCM's. Tat-αBC proteins were applied to cells at 50 μM, 100 μM and 200 μM concentrations to examine does dependent uptake of the proteins. While all three proteins were taken up by the cells, only Tat-αBC wildtype demonstrated a distinct dose dependent uptake (FIG. 25A). To ensure that the tat-αBC proteins were being internalized and were not just coating the surface of the cell, tat-αBC wildtype was visualized by confocal microscopy. Prior to being applied to the NRVCM's, Texas-red fluorophore was covalently attached to the tat-αBC wildtype fusion protein. Following a one hour incubation with the labeled tat-αBC wildtype, NRVCM's were fixed and stained for α-Actinin TOPPRO was used to stain cell nuclei. The texas-red labeled tat-αBC wildtype protein was found throughout the cell, consistent with internalization of the protein (FIG. 25B).

Following confirmation that the proteins were being taken up into the cells, several functional assays were performed with cells that had been treated with tat-αBC proteins (data not shown). However, after experiencing mixed results from these experiments, the integrity of the protein once it entered the cell was in question. Therefore several experiments were undertaken to determine if the tat-αBC proteins were behaving in a similar manner to endogenous αBC.

FIG. 25—Uptake of tat-αBC protein by NRVCM's: Panel A: Western blots examining the uptake of tat-αBC fusion proteins by NRVCM's. NRVCM's were incubated with increasing concentrations of the fusion proteins ranging from 50 nM to 200 nM. Panel B: Confocal analysis of tat-αBC uptake was performed using tat-αBC wildtype covalently linked to texas red fluorophore.

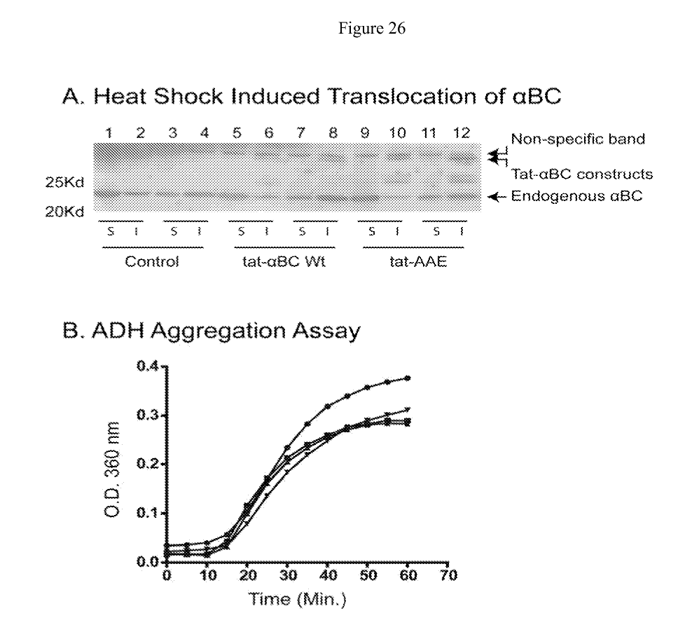

To test if the tat-αBC proteins distributed appropriately within the cell and to see if they responded to stress in a manner similar to endogenous αBC, NRVCM's were incubated with the tat-αBC proteins and subjected to heat shock. Following heat shock the cells were fractionated in buffer with mild detergent and centrifuged at 300 g for 5 minutes. The soluble fraction (supernatant) and the insoluble fraction (pellet) were then analyzed for the presence of αBC by western blotting. Under non-stressed conditions endogenous αBC is found predominantly in the soluble fraction (FIG. 26A lanes 1,5, and 9). However, following heat shock, a majority of endogenous αBC translocates to the insoluble fraction (FIG. 26A lanes 4,8, and 12). On the other hand, the tat-αBC proteins were found exclusively in the insoluble fractions in both unstressed and stressed cells (FIG. 26A lanes 6,8,10, and 12).

There have been quite a few reports that have shown that αBC can protect against the heat induced denaturation of proteins in vitro [153, 173, 208-210]. Therefore, the chaperone activity of the tat-αBC wildtype protein was assessed by examining its ability to prevent heat induced denaturation and aggregation of alcohol dehydrogenase (ADH), a protein that αBC has been previously shown to chaperone. ADH aggregation at 44° C. was unaffected by either BSA (protein control) or tat-αBC wildtype (FIG. 26B).

FIG. 26—Inappropriate partitioning and instability of the tat-αBC proteins: Panel A: NRVCM's were incubated with tat-αBC constructs for 1 hour prior to heat shock. Following heat shock the cell extracts were separated into soluble and insoluble fraction and analyzed by western blot for the presence of endogenous αBC and the tat-αBC constructs. Panel B: ADH aggregation in the presence of either ADH Alone (squares), ADH and BSA (1:4 molar ratio, triangle), ADH and tat-αBC (1:2, upside down triangle) or ADH and tat-αBC (1:4, circles).

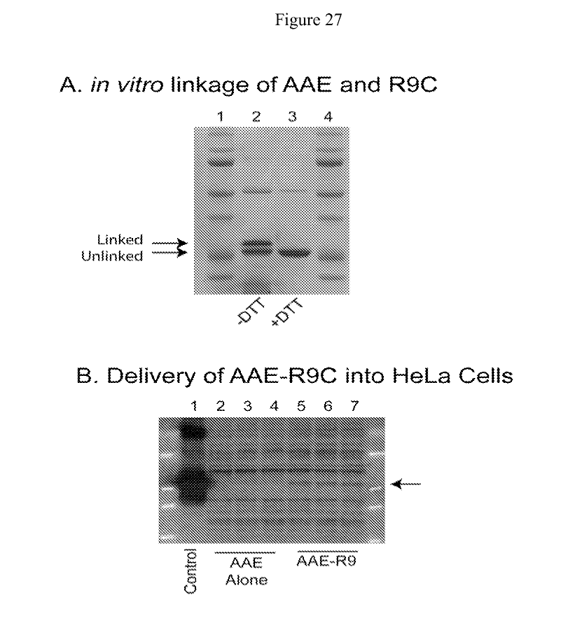

FIG. 27—Linkage of R9-C and AAE and delivery of AAE into HeLa cells: Panel A: The linkage reaction is carried out in vitro, following the 30 minute incubation time a portion of the linkage reaction was treated with or without DTT, separated on SDS-PAGE and visualized by Coomassie stain. Approximately 50% of the AAE was linked with the R9 following the linkage reaction (lane 2). Addition of DTT uncouples the peptide and its cargo (lane 3). Panel B: HeLa cells were incubated for 30 minutes with AAE alone or AAE linked to the R9-C peptide. Cells were collected by trypsinization and analyzed by western blot for the presence of AAE in the cell extract. AAE was found in the extracts of cells incubated with the R9-linked protein (lanes 5-7), but not in the cells incubated with AAE alone (lanes 2-4).

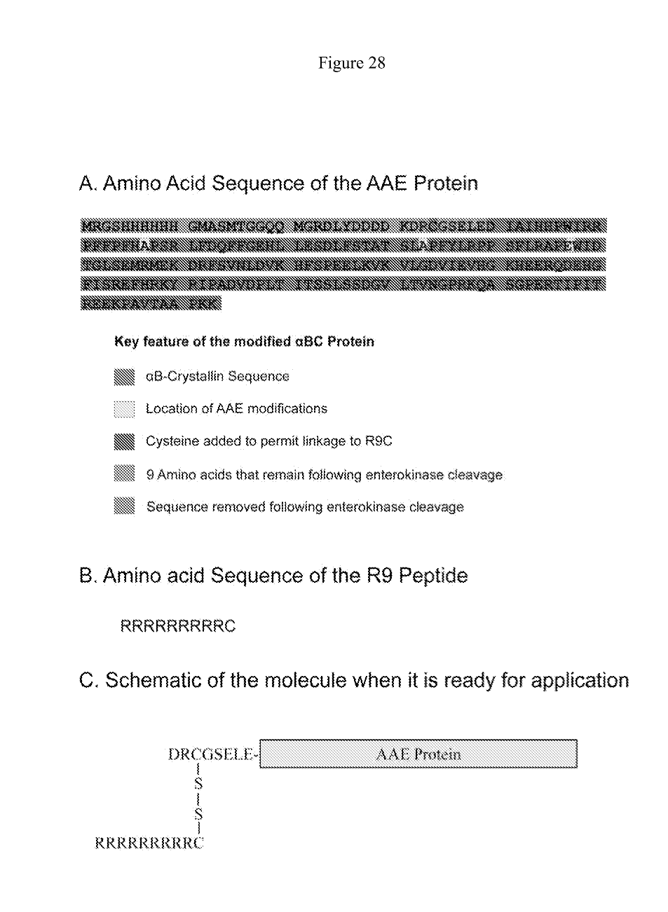

FIG. 28—Design of the R9-C delivery platform: Panel A: Sequence modifications made to the PrSET-αBC construct to facilitate linkage to the R9-C peptide, Panel B: The R9-C peptide. Panel C: A schematic of the molecule following the linkage reaction between R9-C and AAE. FIG. 28A schematically illustrates the amino acid sequence of domains in the exemplary polypeptide of the invention comprising the AAE protein of the invention: including the aB crystalline sequence

(SEQ ID NO: 1)
D IAIHHPWIRR PFFPFHAPSR LFDQFFGEHL LESDLFSTAT
SLAPFYLRPP SFLRAPEWID TGLSEMRMEK DRFSVNLDVK
HFSPEELKVK VLGDVIEVHG KHEERQDEHG FISREFHRKY
RIPADVDPLT ITSSLSSDGV LTVNGPRKQA SGPERTIPIT
REEKPAVTAA PKK,

the location of AAE modifications (the “A” bolded and underlined in the PFFPFHAPSR of SEQ ID NO:1; the “A” bolded and underlined in the SLAPFYLRPP of SEQ ID NO:1; and, the “E” bolded and underlined in the SFLRAPEWID of SEQ ID NO:1),

a cysteine added to permit linkage to R9c (the “C” bolded and underlined in KDRCGSELE, or SEQ ID NO:2),

the nine amino acids that remain following enterokinase cleavage: KDRCGSELE (SEQ ID NO:2), and

the sequence removed following enterokinase cleavage: MRGSHHHHHH GMASMTGGQQ MGRDLYDDDD (SEQ ID NO:3).

FIG. 28B shows the R9 peptide and with a cysteine residue: RRRRRRRRRC (SEQ ID NO:4).

FIG. 28C schematically illustrates this exemplary molecule of the invention ready for application/administration, including peptide DRCGSELE (SEQ ID NO:5) linked by a disulfide bond to peptide RRRRRRRRRC (SEQ ID NO:4).

In alternative embodiments, the invention encompasses polypeptides having alternative forms of αB crystalline sequences with the equivalent amino acid peptide residue substitutions.

Since it appeared that fusing αBC to the tat domain had destabilized the protein and eliminated its chaperone activity a new plan was devised to develop a delivery platform based on CPP transduction which did not require the creation of fusion proteins or covalent linkages. Instead this new system relies on creating a disulfide bond between the CPP and the cargo protein. This disulfide bond should be fairly stable in the weakly reducing environments of cell media and blood. However, the more strongly reducing intracellular environment it should be reduced resulting in release of the cargo protein from the CPP. This strategy has been used successfully with the tat transduction domain previously. For this application a synthetic CPP, R9-C, consisting of 9 arginines and a cysteine, to create the disulfide bond with cargo proteins, will be employed. The R9 motif was selected since it has been demonstrated to have one of the highest protein transduction efficiencies of all the known CPP's.

Since αBC-AAE is known to be the most protective form of αBC, it became the form that was focused on during the development of the αBC-R9-C delivery platform [151]. Alpha-BC does not contain any cysteine residue, therefore, in order make αBC-AAE compatible with this system a cysteine needed to be added to allow formation of the disulfide bond. In order to minimize structural changes that may occur by introducing a cysteine it was placed within the 9 amino acids that remain following enterokinase (EK) cleavage of the purified protein (see FIG. 28A). The R9-C peptide was synthesized and purified in vitro.

Linking the modified αBC-AAE protein and the R9-C peptide is accomplished by incubating the protein and the peptide in equimolar concentrations in 50 mM Tris pH 7.4 at room temperature for 30 minutes (FIG. 27A, lane 2). To demonstrate that the linkage is sensitive to reduction, 5 mM dithiotheritol (DTT) was added at the end of the reaction and resulted in elimination of the linked molecule (FIG. 27A, lane 3). Since it had been successfully demonstrated that the linkage between αBC-AAE and the R9-C peptide could be formed, the ability of the linked molecule to deliver αBC-AAE was tested in HeLa cells. Cells were incubated with 100 μM concentration of the linked protein for 30 minutes, following the incubation cells were trypsinized and collected. Delivery of αBC-AAE by the R9-C peptide was confirmed by western blotting (FIG. 27B, lanes 5-7). Delivery of αBC-AAE was dependent on the presence of the R9-C peptide as shown by the failure of the αBC-AAE protein alone to enter the cell (FIG. 27B, lanes 2-4).

### 4. Discussion

The invention provides systems for delivering not only αBC-AAE, but other proteins and even small molecules to cells in culture as well as organs in both ex vivo and in vivo settings. It has been demonstrated that the linkage can be made between the R9-C peptide and, in this case, αBC-AAE, and that the linked protein is deliverable to cells, e.g., HeLa cells.

The most popular method for delivering proteins via CPP's has been to create fusion proteins consisting of the CPP and the protein of interest. However, this method can be unreliable as the addition of the CPP sequence and other related sequences can alter protein folding leading to destabilization and loss of function in the protein of interest. The results from the studies utilizing the tat-αBC fusion proteins are a prime example. From discussions with others in the field that have experience with CPP fusion proteins, this is common and only a third of CPP fusion proteins that they have created functioned as expected.

The system created here is unique due to the placement of the cysteine in the sequence downstream from the EK site, but upstream of the multiple cloning site in the vector. The expression vector can now be used to make any protein compatible with this system. This creates a powerful tool for both research and the development of therapeutics as it becomes simple to insert proteins into the vector, express and purify them and link them to the CPP peptide. What we have created is essentially a modular system for the transduction of proteins across cell membranes. The combination of CPP peptide and cargo proteins can be mixed and matched nearly effortlessly to find the right combination that provides the desired result. Continued development of this package could lead to several interesting research and therapeutic tools.

Presented here are two studies that demonstrate the efficacy of polypeptides of this invention and define two new roles for αBC in protecting the myocardium. The first study demonstrates that during ischemia, αBC translocates from the cytosol to the mitochondria resulting in the inhibition of cytochrome c release, possibly through interactions with VDAC and BAX. The second study establishes a role for αBC in supporting glutathione recycling in a manner that protects cells from oxidative stress. The results from both studies demonstrated that αBC inhibits apoptotic signaling.

The intraorganellar environments vary from organelle to organelle, for instance, the redox status of the ER is shifted toward a more oxidizing environment, presumably to assist in folding proteins by inhibiting unfavorable disulfide bond formations. However, in contrast to the ER, mitochondria are believed to be shifted towards a more reducing environment. The different environments are established by the redox machinery and the reducing equivalents present within the organelle. The same glutathione recycling components are present within each organelle, but due to the different environments, there are differences in the protein levels and activities of the components.

Given, the findings in the two studies discussed above, the question rises as to whether αBC, when it translocates to the mitochondria during stress, affects the redox status of the mitochondria through its ability to enhance GR activity. The data demonstrate that αBC may exist within the mitochondria, which suggests that it would have the ability to interact with intramitochondrial GR and, perhaps, increase glutathione recycling, shifting the redox status towards a more reducing environment. Moreover, glutathione import is stimulated by malate and pyruvate [211]. Interestingly, the MudPIT analysis found that mitochondrial αBC co-immunoprecipitated with a malate carrier protein.

To add yet another layer of complexity are the preliminary findings that αBC may play a role in regulating metabolism. The MudPIT analysis of mitochondrial αBC immunoprecipitations identified several members of the electron transport chain as well as proteins involved in β-oxidation. The electron transport chain proteins identified are located within complex I and complex V. Ivor Benjamin's lab has reported reduced complex I activity in αBC/HSPB2 knockout mice [212]. Furthermore, when they examined skinned fibers from the same knockout mice, they found reductions in ATP that were much larger than would be expected for the changes observed in their mitochondrial respiration studies. This may suggest that ATP synthase is affected by the loss of αBC; our preliminary MudPIT results show that αBC co-immuno-precipitates with at least two ATP synthase subunits. The effects of αBC on mitochondrial respiration definitely warrant further examination. The interactions with VDAC, beyond possible inhibiting cytochrome c release, may also play role in regulating mitochondrial metabolism.

In addition to examining these new roles for αBC, the possibility that the R9-C, CPP based, protein transduction system could be developed as a research tool, or perhaps even a therapeutic, deserves further examination. This system, or one similar to it, may prove invaluable in harnessing the protective effects of proteins, such as αBC. Since it lies downstream of several complex signaling pathways, modulation of αBC levels and/or phosphorylation status may not lend itself well to traditional small molecule drugs. Additionally, the effects of small molecule drugs can take hours or days to occur. This is well beyond the time frame of the early, damaging events of acute ischemic injury; however, CPP transduction has been shown to occur rapidly with uptake of tat-βgal into the mouse heart occurring within 15 minutes of delivery via intraperitoneal injection, which is within the therapeutic window to minimize myocyte cell death during MI [207]. The literature suggests that αBC may be one of the most powerful pro-survival factors of the heart during I/R. However, there is a large gap between being able to recognize a protective agent and being able to harness its power, hopefully, either R9-C or some other technology will one day be able to bridge that gap.

### REFERENCES

1. Rosamond, W., et al., Heart Disease and Stroke Statistics—2008 Update: A Report From the American Heart Association Statistics Committee and Stroke Statistics Subcommittee 10.1161/CIRCULATIONAHA.107.187998. Circulation, 2008. 117(4): p. e25-146.
2. Sack, M. N., Mitochondrial depolarization and the role of uncoupling proteins in ischemia tolerance 10.1016/j.cardiores.2006.07.010. Cardiovasc Res, 2006. 72(2): p. 210-219.
3. Zucchi, R., S. Ghelardoni, and S. Evangelista, Biochemical basis of ischemic heart injury and of cardioprotective interventions. Curr Med Chem, 2007. 14(15): p. 1619-37.
4. Opie, L. H., Effects of regional ischemia on metabolism of glucose and fatty acids. Relative rates of aerobic and anaerobic energy production during myocardial infarction and comparison with effects of anoxia. Circ Res, 1976. 38(5 Suppl 1): p. 152-74.
5. Graham, R. M., et al., A unique pathway of cardiac myocyte death caused by hypoxia-acidosis. J Exp Biol, 2004. 207 (Pt 18): p. 3189-200.
6. Allen, D. G. and X.-H. Xiao, Role of the cardiac Na+/H+ exchanger during ischemia and reperfusion 10.1016/S0008-6363(02)00836-2. Cardiovasc Res, 2003. 57(4): p. 934-941.
7. MORRIS KARMAZYN, The Role of the Myocardial Sodium-Hydrogen Exchanger in Mediating Ischemic and Reperfusion Injury: From Amiloride to Cariporide<sup>a</sup>. Annals of the New York Academy of Sciences, 1999. 874 (HEART IN STRESS): p. 326-334.
8. Piper, H. M., Y. Abdallah, and C. Schafer, The first minutes of reperfusion: a window of opportunity for cardioprotection. Cardiovasc Res, 2004. 61(3): p. 365-71.
9. Gustafsson, A. B. and R. A. Gottlieb, Heart mitochondria: gates of life and death 10.1093/cvr/cvm005. Cardiovasc Res, 2008. 77(2): p. 334-343.
10. Aggeli, I. K., I. Beis, and C. Gaitanaki, Oxidative stress and calpain inhibition induce alpha B-crystallin phosphorylation via p38-MAPK and calcium signalling pathways in H9c2 cells. Cell Signal, 2008. 20(7): p. 1292-302.
11. Singh, R. B., et al., Role of proteases in the pathophysiology of cardiac disease. Mol Cell Biochem, 2004. 263(1-2): p. 241-56.
12. Bers, D. M., Cardiac excitation-contraction coupling. Nature, 2002. 415(6868): p. 198-205.
13. Tissier, R., et al., Making the heart resistant to infarction: how can we further decrease infarct size? Front Biosci, 2008. 13: p. 284-301.
14. Ambrosio, G. and M. Chiariello, Myocardial reperfusion injury: mechanisms and management—a review. Am J Med, 1991. 91(3C): p. 86S-88S.
15. Kadenbach, B., Intrinsic and extrinsic uncoupling of oxidative phosphorylation. Biochim Biophys Acta, 2003. 1604(2): p. 77-94.
16. Dhalla, N. S., et al., Status of myocardial antioxidants in ischemia-reperfusion injury. Cardiovasc Res, 2000. 47(3): p. 446-56.
17. Marczin, N., et al., Antioxidants in myocardial ischemia-reperfusion injury: therapeutic potential and basic mechanisms. Arch Biochem Biophys, 2003. 420(2): p. 222-36.
18. Zorov, D. B., et al., Reactive oxygen species (ROS)-induced ROS release: a new phenomenon accompanying induction of the mitochondrial permeability transition in cardiac myocytes. J Exp Med, 2000. 192(7): p. 1001-14.
19. Slodzinski, M. K., M. A. Aon, and B. O'Rourke, Glutathione oxidation as a trigger of mitochondrial depolarization and oscillation in intact hearts. J Mol Cell Cardiol, 2008. 45(5): p. 650-60.
20. Zweier, J. L., J. T. Flaherty, and M. L. Weisfeldt, Direct measurement of free radical generation following reperfusion of ischemic myocardium. Proc Natl Acad Sci U S A, 1987. 84(5): p. 1404-7.
21. Sandeep Raha, B. H. R., Mitochondria, oxygen free radicals, and apoptosis. American Journal of Medical Genetics, 2001. 106(1): p. 62-70.
22. Ho, Y. S., et al., The nature of antioxidant defense mechanisms: a lesson from transgenic studies. Environ Health Perspect, 1998. 106 Suppl 5: p. 1219-28.
23. Abunasra, H. J., et al., Efficacy of adenoviral gene transfer with manganese superoxide dismutase and endothelial nitric oxide synthase in reducing ischemia and reperfusion injury. Eur J Cardiothorac Surg, 2001. 20(1): p. 153-8.
24. Haramaki, N., et al., Networking antioxidants in the isolated rat heart are selectively depleted by ischemia-reperfusion. Free Radic Biol Med, 1998. 25(3): p. 329-39.
25. Leichtweis, S, and L. L. Ji, Glutathione deficiency intensifies ischaemia-reperfusion induced cardiac dysfunction and oxidative stress. Acta Physiol Scand, 2001. 172(1): p. 1-10.
26. Singh, A., et al., Relation between myocardial glutathione content and extent of ischemia-reperfusion injury. Circulation, 1989. 80(6): p. 1795-804.
27. Ramires, P. R. and L. L. Ji, Glutathione supplementation and training increases myocardial resistance to ischemia-reperfusion in vivo. Am J Physiol Heart Circ Physiol, 2001. 281(2): p. H679-88.
28. Li, S., X. Li, and G. J. Rozanski, Regulation of glutathione in cardiac myocytes. J Mol Cell Cardiol, 2003. 35(9): p. 1145-52.
29. Antunes, F., D. Han, and E. Cadenas, Relative contributions of heart mitochondria glutathione peroxidase and catalase to H(2)O(2) detoxification in in vivo conditions. Free Radic Biol Med, 2002. 33(9): p. 1260-7.
30. Anderson, M. E., Determination of glutathione and glutathione disulfide in biological samples. Methods Enzymol, 1985. 113: p. 548-55.
31. Ferrari, R., et al., Oxidative stress during myocardial ischaemia and heart failure. Curr Pharm Des, 2004. 10(14): p. 1699-711.
32. Mansfield, K. D., M. C. Simon, and B. Keith, Hypoxic reduction in cellular glutathione levels requires mitochondrial reactive oxygen species. J Appl Physiol, 2004. 97(4): p. 1358-66.
33. Forman, H. J., H. Zhang, and A. Rinna, Glutathione: Overview of its protective roles, measurement, and biosynthesis. Mol Aspects Med, 2008.
34. Homolya, L., A. Varadi, and B. Sarkadi, Multidrug resistance-associated proteins: Export pumps for conjugates with glutathione, glucuronate or sulfate. Biofactors, 2003. 17(1-4): p. 103-14.
35. Venardos, K. M., et al., Myocardial ischemia-reperfusion injury, antioxidant enzyme systems, and selenium: a review. Curr Med Chem, 2007. 14(14): p. 1539-49.
36. Jain, M., et al., Increased myocardial dysfunction after ischemia-reperfusion in mice lacking glucose-6-phosphate dehydrogenase. Circulation, 2004. 109(7): p. 898-903.
37. Leopold, J. A., et al., Glucose-6-phosphate dehydrogenase overexpression decreases endothelial cell oxidant stress and increases bioavailable nitric oxide. Arterioscler Thromb Vasc Biol, 2003. 23(3): p. 411-7.
38. Van Remmen, H., et al., Multiple deficiencies in antioxidant enzymes in mice result in a compound increase in sensitivity to oxidative stress. Free Radic Biol Med, 2004. 36(12): p. 1625-34.
39. Copin, J.-C., Y. Gasche, and P. H. Chan, Overexpression of copper/zinc superoxide dismutase does not prevent neonatal lethality in mutant mice that lack manganese superoxide dismutase. Free Radical Biology and Medicine, 2000. 28(10): p. 1571-1576.
40. Chen, Z., et al., Overexpression of MnSOD protects against myocardial ischemia/reperfusion injury in transgenic mice. J Mol Cell Cardiol, 1998. 30(11): p. 2281-9.
41. Shiomi, T., et al., Overexpression of glutathione peroxidase prevents left ventricular remodeling and failure after myocardial infarction in mice. Circulation, 2004. 109(4): p. 544-9.
42. Hoshida, S., et al., Ebselen protects against ischemia-reperfusion injury in a canine model of myocardial infarction. Am J Physiol, 1994. 267(6 Pt 2): p. H2342-7.
43. Chance, B. and N. Oshino, Kinetics and mechanisms of catalase in peroxisomes of the mitochondrial fraction. Biochem J, 1971. 122(2): p. 225-33.
44. Vainshtein, B. K., et al., Three-dimensional structure of the enzyme catalase. Nature, 1981. 293(5831): p. 411-2.
45. Li, G., et al., Catalase-overexpressing transgenic mouse heart is resistant to ischemia-reperfusion injury. Am J Physiol, 1997. 273(3 Pt 2): p. H1090-5.
46. World, C. J., H. Yamawaki, and B. C. Berk, Thioredoxin in the cardiovascular system. J Mol Med, 2006. 84(12): p. 997-1003.
47. Tao, L., et al., Nitrative Inactivation of Thioredoxin-1 and Its Role in Postischemic Myocardial Apoptosis 10.1161/CIRCULATIONAHA.106.625061. Circulation, 2006. 114(13): p. 1395-1402.
48. Tao, L., et al., Cardioprotective effects of thioredoxin in myocardial ischemia and reperfusion: role of S-nitrosation [corrected]. Proc Natl Acad Sci USA, 2004. 101(31): p. 11471-6.
49. Yamamoto, M., et al., Inhibition of endogenous thioredoxin in the heart increases oxidative stress and cardiac hypertrophy. J Clin Invest, 2003. 112(9): p. 1395-406.
50. Shioji, K., et al., Overexpression of thioredoxin-1 in transgenic mice attenuates adriamycin-induced cardiotoxicity. Circulation, 2002. 106(11): p. 1403-9.
51. Turoczi, T., et al., Thioredoxin redox signaling in the ischemic heart: an insight with transgenic mice overexpressing Trx1. J Mol Cell Cardiol, 2003. 35(6): p. 695-704.
52. Nishida, K. and K. Otsu, The role of apoptosis signal-regulating kinase 1 in cardiomyocyte apoptosis. Antioxid Redox Signal, 2006. 8(9-10): p. 1729-36.
53. Ago, T. and J. Sadoshima, Thioredoxin1 as a negative regulator of cardiac hypertrophy. Antioxid Redox Signal, 2007. 9(6): p. 679-87.
54. Guaiquil, V. H., et al., Vitamin C inhibits hypoxia-induced damage and apoptotic signaling pathways in cardiomyocytes and ischemic hearts. Free Radic Biol Med, 2004. 37(9): p. 1419-29.
55. Shinke, T., et al., Vitamin C restores the contractile response to dobutamine and improves myocardial efficiency in patients with heart failure after anterior myocardial infarction. Am Heart J, 2007. 154(4): p. 645 e1-8.
56. Zhou, H., et al., Vitamin C pretreatment attenuates hypoxia-induced disturbance of sodium currents in guinea pig ventricular myocytes. J Membr Biol, 2006. 211(2): p. 81-7.
57. Sesso, H. D., et al., Vitamins E and C in the prevention of cardiovascular disease in men: the Physicians' Health Study II randomized controlled trial. Jama, 2008. 300(18): p. 2123-33.
58. Ueda, S, and K. Yasunari, What we learnt from randomized clinical trials and cohort studies of antioxidant vitamin? Focus on vitamin E and cardiovascular disease. Curr Pharm Biotechnol, 2006. 7(2): p. 69-72.
59. Matsui, T. and A. Rosenzweig, Convergent signal transduction pathways controlling cardiomyocyte survival and function: the role of PI 3-kinase and Akt. J Mol Cell Cardiol, 2005. 38(1): p. 63-71.
60. Mullonkal, C. J. and L. H. Toledo-Pereyra, Akt in ischemia and reperfusion. J Invest Surg, 2007. 20(3): p. 195-203.
61. Chiari, P. C., et al., Isoflurane protects against myocardial infarction during early reperfusion by activation of phosphatidylinositol-3-kinase signal transduction: evidence for anesthetic-induced postconditioning in rabbits. Anesthesiology, 2005. 102(1): p. 102-9.
62. Takada, Y., et al., Cytoprotective effect of sodium orthovanadate on ischemia/reperfusion-induced injury in the rat heart involves Akt activation and inhibition of fodrin breakdown and apoptosis. J Pharmacol Exp Ther, 2004. 311(3): p. 1249-55.
63. Fujio, Y., et al., Akt promotes survival of cardiomyocytes in vitro and protects against ischemia-reperfusion injury in mouse heart. Circulation, 2000. 101(6): p. 660-7.
64. Bhuiyan, M. S., N. Shioda, and K. Fukunaga, Targeting protein kinase B/Akt signaling with vanadium compounds for cardioprotection. Expert Opin Ther Targets, 2008. 12(10): p. 1217-27.
65. Matsui, T., et al., Akt activation preserves cardiac function and prevents injury after transient cardiac ischemia in vivo. Circulation, 2001. 104(3): p. 330-5.
66. Budas, G. R., E. N. Churchill, and D. Mochly-Rosen, Cardioprotective mechanisms of PKC isozyme-selective activators and inhibitors in the treatment of ischemia-reperfusion injury. Pharmacol Res, 2007. 55(6): p. 523-36.
67. Barnett, M. E., D. K. Madgwick, and D. J. Takemoto, Protein kinase C as a stress sensor. Cell Signal, 2007. 19(9): p. 1820-9.
68. Churchill, E. N. and D. Mochly-Rosen, The roles of PKCdelta and epsilon isoenzymes in the regulation of myocardial ischaemia/reperfusion injury. Biochem Soc Trans, 2007. 35(Pt 5): p. 1040-2.
69. Chen, L., et al., Opposing cardioprotective actions and parallel hypertrophic effects of delta PKC and epsilon PKC. Proc Natl Acad Sci USA, 2001. 98(20): p. 11114-9.
70. Hahn, H. S., et al., Ischemic protection and myofibrillar cardiomyopathy: dose-dependent effects of in vivo deltaPKC inhibition. Circ Res, 2002. 91(8): p. 741-8.
71. Inagaki, K., et al., Inhibition of delta-protein kinase C protects against reperfusion injury of the ischemic heart in vivo. Circulation, 2003. 108(19): p. 2304-7.
72. Teng, J. C., et al., Mechanisms related to the cardioprotective effects of protein kinase C epsilon (PKC epsilon) peptide activator or inhibitor in rat ischemia/reperfusion injury. Naunyn Schmiedebergs Arch Pharmacol, 2008. 378(1): p. 1-15.
73. Yue, Y., Y. Qu, and M. Boutjdir, Protective role of protein kinase C epsilon activation in ischemia-reperfusion arrhythmia. Biochem Biophys Res Commun, 2006. 349(1): p. 432-8.
74. Baines, C. P., et al., Protein kinase Cepsilon interacts with and inhibits the permeability transition pore in cardiac mitochondria. Circ Res, 2003. 92(8): p. 873-80.
75. Miyamae, M., et al., Activation of epsilon protein kinase C correlates with a cardioprotective effect of regular ethanol consumption. Proc Natl Acad Sci USA, 1998. 95(14): p. 8262-7.
76. Armstrong, S. C., Protein kinase activation and myocardial ischemia/reperfusion injury. Cardiovasc Res, 2004. 61(3): p. 427-36.
77. Baines, C. P. and J. D. Molkentin, STRESS signaling pathways that modulate cardiac myocyte apoptosis. J Mol Cell Cardiol, 2005. 38(1): p. 47-62.
78. Wang, Y., Mitogen-activated protein kinases in heart development and diseases. Circulation, 2007. 116(12): p. 1413-23.
79. Li, D. Y., et al., Role of ERK1/2 in the anti-apoptotic and cardioprotective effects of nitric oxide after myocardial ischemia and reperfusion. Apoptosis, 2006. 11(6): p. 923-30.
80. Sugden, P. H. and A. Clerk, Oxidative stress and growth-regulating intracellular signaling pathways in cardiac myocytes. Antioxid Redox Signal, 2006. 8(11-12): p. 2111-24.
81. Yue, T. L., et al., Inhibition of extracellular signal-regulated kinase enhances Ischemia/Reoxygenation-induced apoptosis in cultured cardiac myocytes and exaggerates reperfusion injury in isolated perfused heart. Circ Res, 2000. 86(6): p. 692-9.
82. Hausenloy, D. J. and D. M. Yellon, New directions for protecting the heart against ischaemia-reperfusion injury: targeting the Reperfusion Injury Salvage Kinase (RISK)-pathway. Cardiovasc Res, 2004. 61(3): p. 448-60.
83. Wang, Y. F., et al., Apoptosis induction in human melanoma cells by inhibition of MEK is caspase-independent and mediated by the Bcl-2 family members PUMA, Bim, and Mcl-1. Clin Cancer Res, 2007. 13(16): p. 4934-42.
84. Duplain, H., Salvage of ischemic myocardium: a focus on JNK. Adv Exp Med Biol, 2006. 588: p. 157-64.
85. Matsukawa, J., et al., The ASK1-MAP kinase cascades in mammalian stress response. J Biochem, 2004. 136(3): p. 261-5.
86. Wang, X., A. Destrument, and C. Tournier, Physiological roles of MKK4 and MKK7: insights from animal models. Biochim Biophys Acta, 2007. 1773(8): p. 1349-57.
87. Cook, S. A., P. H. Sugden, and A. Clerk, Activation of c-Jun N-terminal kinases and p38-mitogen-activated protein kinases in human heart failure secondary to ischaemic heart disease. J Mol Cell Cardiol, 1999. 31(8): p. 1429-34.
88. Li, W. G., et al., Antioxidant therapy attenuates JNK activation and apoptosis in the remote noninfarcted myocardium after large myocardial infarction. Biochem Biophys Res Commun, 2001. 280(1): p. 353-7.
89. Dougherty, C. J., et al., Mitochondrial signals initiate the activation of c-Jun N-terminal kinase (JNK) by hypoxia-reoxygenation. Faseb J, 2004. 18(10): p. 1060-70.
90. Ferrandi, C., et al., Inhibition of c-Jun N-terminal kinase decreases cardiomyocyte apoptosis and infarct size after myocardial ischemia and reperfusion in anaesthetized rats. Br J Pharmacol, 2004. 142(6): p. 953-60.
91. Kaiser, R. A., et al., Genetic inhibition or activation of JNK1/2 protects the myocardium from ischemia-reperfusion-induced cell death in vivo. J Biol Chem, 2005. 280(38): p. 32602-8.
92. Petrich, B. G., J. D. Molkentin, and Y. Wang, Temporal activation of c-Jun N-terminal kinase in adult transgenic heart via cre-loxP-mediated DNA recombination. FASEB J, 2003. 17(6): p. 749-51.
93. Han, J. Y., et al., C-jun N-terminal kinase regulates the interaction between 14-3-3 and Bad in ethanol-induced cell death. J Neurosci Res, 2008. 86(14): p. 3221-9.
94. Zhang, J., et al., BAD Ser128 is not phosphorylated by c-Jun NH2-terminal kinase for promoting apoptosis. Cancer Res, 2005. 65(18): p. 8372-8.
95. Sunayama, J., et al., JNK antagonizes Akt-mediated survival signals by phosphorylating 14-3-3. J Cell Biol, 2005. 170(2): p. 295-304.
96. Donovan, N., et al., JNK phosphorylation and activation of BAD couples the stress-activated signaling pathway to the cell death machinery. J Biol Chem, 2002. 277(43): p. 40944-9.
97. Ono, K. and J. Han, The p38 signal transduction pathway: activation and function. Cell Signal, 2000. 12(1): p. 1-13.
98. Martindale, J. J., et al., Overexpression of mitogen-activated protein kinase kinase 6 in the heart improves functional recovery from ischemia in vitro and protects against myocardial infarction in vivo. J Biol Chem, 2005. 280(1): p. 669-76.
99. Wang, X. S., et al., MAPKKK6, a novel mitogen-activated protein kinase kinase kinase, that associates with MAPKKK5. Biochem Biophys Res Commun, 1998. 253(1): p. 33-7.
100. Steenbergen, C., The role of p38 mitogen-activated protein kinase in myocardial ischemia/reperfusion injury; relationship to ischemic preconditioning. Basic Res Cardiol, 2002. 97(4): p. 276-85.
101. Taniike, M., et al., Apoptosis signal-regulating kinase 1/p38 signaling pathway negatively regulates physiological hypertrophy. Circulation, 2008. 117(4): p. 545-52.
102. Bassi, R., et al., Targeting p38-MAPK in the ischaemic heart: kill or cure? Curr Opin Pharmacol, 2008. 8(2): p. 141-6.
103. Barancik, M., et al., Inhibition of the cardiac p38-MAPK pathway by SB203580 delays ischemic cell death. J Cardiovasc Pharmacol, 2000. 35(3): p. 474-83.
104. Kaiser, R. A., et al., Inhibition of p38 reduces myocardial infarction injury in the mouse but not pig after ischemia-reperfusion. Am J Physiol Heart Circ Physiol, 2005. 289(6): p. H2747-51.
105. Andrews, C., et al., The MKK6-p38 MAPK pathway prolongs the cardiac contractile calcium transient, downregulates SERCA2, and activates NF-AT. Cardiovasc Res, 2003. 59(1): p. 46-56.
106. Liao, P., et al., p38 Mitogen-activated protein kinase mediates a negative inotropic effect in cardiac myocytes. Circ Res, 2002. 90(2): p. 190-6.
107. Mackay, K. and D. Mochly-Rosen, Involvement of a p38 mitogen-activated protein kinase phosphatase in protecting neonatal rat cardiac myocytes from ischemia. J Mol Cell Cardiol, 2000. 32(8): p. 1585-8.
108. Jin, J. K., et al., Localization of phosphorylated alphaB-crystallin to heart mitochondria during ischemia-reperfusion. Am J Physiol Heart Circ Physiol, 2008. 294(1): p. H337-44.
109. Wall, J. A., et al., Alterations in oxidative phosphorylation complex proteins in the hearts of transgenic mice that overexpress the p38 MAP kinase activator, MAP kinase kinase 6. Am J Physiol Heart Circ Physiol, 2006. 291(5): p. H2462-72.
110. Moolman, J. A., et al., Inhibition of myocardial apoptosis by ischaemic and beta-adrenergic preconditioning is dependent on p38 MAPK. Cardiovasc Drugs Ther, 2006. 20(1): p. 13-25.
111. Hoover, H. E., et al., alpha B-crystallin gene induction and phosphorylation by MKK6-activated p38. A potential role for alpha B-crystallin as a target of the p38 branch of the cardiac stress response. J Biol Chem, 2000. 275(31): p. 23825-33.
112. Craig, R., et al., p38 MAPK and NF-kappa B collaborate to induce interleukin-6 gene expression and release. Evidence for a cytoprotective autocrine signaling pathway in a cardiac myocyte model system. J Biol Chem, 2000. 275(31): p. 23814-24.
113. Li, G., I. S. Ali, and R. W. Currie, Insulin-induced myocardial protection in isolated ischemic rat hearts requires p38 MAPK phosphorylation of Hsp27. Am J Physiol Heart Circ Physiol, 2008. 294(1): p. H74-87.
114. Peart, J. N., et al., Impaired p38 MAPK/HSP27 signaling underlies aging-related failure in opioid-mediated cardioprotection. J Mol Cell Cardiol, 2007. 42(5): p. 972-80.
115. Hartl, F. U. and M. Hayer-Hartl, Molecular chaperones in the cytosol: from nascent chain to folded protein. Science, 2002. 295(5561): p. 1852-8.
116. Delogu, G., et al., Heat shock proteins and their role in heart injury. Curr Opin Crit. Care, 2002. 8(5): p. 411-6.
117. Kim, Y. K., et al., Deletion of the inducible 70-kDa heat shock protein genes in mice impairs cardiac contractile function and calcium handling associated with hypertrophy. Circulation, 2006. 113(22): p. 2589-97.
118. Hayashi, M., et al., A crucial role of mitochondrial Hsp40 in preventing dilated cardiomyopathy. Nat Med, 2006. 12(1): p. 128-32.
119. Williamson, C. L., et al., Mitochondria protection from hypoxia/reoxygenation injury with mitochondria heat shock protein 70 overexpression. Am J Physiol Heart Circ Physiol, 2008. 294(1): p. H249-56.
120. Fu, H. Y., et al., Overexpression of endoplasmic reticulum-resident chaperone attenuates cardiomyocyte death induced by proteasome inhibition. Cardiovasc Res, 2008. 79(4): p. 600-10.
121. Yan, L. J., et al., Mouse heat shock transcription factor 1 deficiency alters cardiac redox homeostasis and increases mitochondrial oxidative damage. EMBO J, 2002. 21(19): p. 5164-72.
122. Taylor, R. P. and I. J. Benjamin, Small heat shock proteins: a new classification scheme in mammals. J Mol Cell Cardiol, 2005. 38(3): p. 433-44.
123. Fan, G. C., et al., Novel cardioprotective role of a small heat-shock protein, Hsp20, against ischemia/reperfusion injury. Circulation, 2005. 111(14): p. 1792-9.
124. Hollander, J. M., et al., Overexpression of wild-type heat shock protein 27 and a nonphosphorylatable heat shock protein 27 mutant protects against ischemia/reperfusion injury in a transgenic mouse model. Circulation, 2004. 110(23): p. 3544-52.
125. Woods, A. C., E. L. Burky, and M. B. Woodhall, The Organ Specific Properties and Antigenic Power in the Homologous Species of Alpha Crystallin. Trans Am Ophthalmol Soc, 1931. 29: p. 168-73.
126. Roy, D. and A. Spector, Human alpha-crystallin: characterization of the protein isolated from the periphery of cataractous lenses. Biochemistry, 1976. 15(5): p. 1180-8.
127. Quax-Jeuken, Y., et al., Complete structure of the alpha B-crystallin gene: conservation of the exon-intron distribution in the two nonlinked alpha-crystallin genes. Proc Natl Acad Sci USA, 1985. 82(17): p. 5819-23.
128. Dubin, R. A., E. F. Wawrousek, and J. Piatigorsky, Expression of the murine alpha B-crystallin gene is not restricted to the lens. Mol Cell Biol, 1989. 9(3): p. 1083-91.
129. Longoni, S., P. James, and M. Chiesi, Cardiac alpha-crystallin. I. Isolation and identification. Mol Cell Biochem, 1990. 99(1): p. 113-20.
130. NCBI, Entrez Nucleotide. 2008.
131. NCBI, Entrez Protein. 2008.
132. Frederikse, P. H., et al., Structure and alternate tissue preferred transcription initiation of the mouse alpha B-crystallin/small heat shock protein gene. Nucleic Acids Res, 1994. 22(25): p. 5686-94.
133. Gopal-Srivastava, R., J. I. Haynes, 2nd, and J. Piatigorsky, Regulation of the murine alpha B-crystallin/small heat shock protein gene in cardiac muscle. Mol Cell Biol, 1995. 15(12): p. 7081-90.
134. Aoyama, A., et al., Alpha B-crystallin expression in mouse NIH 3T3 fibroblasts: glucocorticoid responsiveness and involvement in thermal protection. Mol Cell Biol, 1993. 13(3): p. 1824-35.
135. Swamynathan, S. K. and J. Piatigorsky, Regulation of the mouse alphaB-crystallin and MKBP/HspB2 promoter activities by shared and gene specific intergenic elements: the importance of context dependency. Int J Dev Biol, 2007. 51(8): p. 689-700.
136. Duncan, B. and K. Zhao, HMGA1 mediates the activation of the CRYAB promoter by BRG1. DNA Cell Biol, 2007. 26(10): p. 745-52.
137. Pasta, S. Y., et al., Role of the conserved SRLFDQFFG region of alpha-crystallin, a small heat shock protein. Effect on oligomeric size, subunit exchange, and chaperone-like activity. J Biol Chem, 2003. 278(51): p. 51159-66.
138. Pasta, S. Y., et al., The IXI/V motif in the C-terminal extension of alpha-crystallins: alternative interactions and oligomeric assemblies. Mol V is, 2004. 10: p. 655-62.
139. Gupta, R. and O. P. Srivastava, Effect of deamidation of asparagine 146 on functional and structural properties of human lens alphaB-crystallin. Invest Ophthalmol V is Sci, 2004. 45(1): p. 206-14.
140. Mehlen, P., et al., Tumor necrosis factor-alpha induces changes in the phosphorylation, cellular localization, and oligomerization of human hsp27, a stress protein that confers cellular resistance to this cytokine. J Cell Biochem, 1995. 58(2): p. 248-59.
141. Ito, H., et al., Phosphorylation-induced change of the oligomerization state of alpha B-crystallin. J Biol Chem, 2001. 276(7): p. 5346-52.
142. Aquilina, J. A., et al., Phosphorylation of alphaB-crystallin alters chaperone function through loss of dimeric substructure. J Biol Chem, 2004. 279(27): p. 28675-80.
143. Ahmad, M. F., et al., Effect of phosphorylation on alpha B-crystallin: differences in stability, subunit exchange and chaperone activity of homo and mixed oligomers of alpha B-crystallin and its phosphorylation-mimicking mutant. J Mol Biol, 2008. 375(4): p. 1040-51.
144. Shroff, N. P., et al., Substituted hydrophobic and hydrophilic residues at methionine-68 influence the chaperone-like function of alphaB-crystallin. Mol Cell Biochem, 2001. 220(1-2): p. 127-33.
145. Muchowski, P. J., et al., Site-directed mutations within the core “alpha-crystallin” domain of the small heat-shock protein, human alphaB-crystallin, decrease molecular chaperone functions. J Mol Biol, 1999. 289(2): p. 397-411.
146. Kumar, L. V., T. Ramakrishna, and C. M. Rao, Structural and functional consequences of the mutation of a conserved arginine residue in alphaA and alphaB crystallins. J Biol Chem, 1999. 274(34): p. 24137-41.
147. Hayes, V. H., G. Devlin, and R. A. Quinlan, Truncation of alphaB-crystallin by the myopathy-causing Q151X mutation significantly destabilizes the protein leading to aggregate formation in transfected cells. J Biol Chem, 2008. 283(16): p. 10500-12.
148. Abraham, E. C., et al., Role of the specifically targeted lysine residues in the glycation dependent loss of chaperone activity of alpha A- and alpha B-crystallins. Mol Cell Biochem, 2008. 310(1-2): p. 235-9.
149. Ito, H., et al., AlphaB-crystallin in the rat lens is phosphorylated at an early post-natal age. FEBS Lett, 1999. 446(2-3): p. 269-72.
150. Ito, H., et al., Phosphorylation of alphaB-crystallin in response to various types of stress. J Biol Chem, 1997. 272(47): p. 29934-41.
151. Morrison, L. E., et al., Mimicking phosphorylation of alphaB-crystallin on serine-59 is necessary and sufficient to provide maximal protection of cardiac myocytes from apoptosis. Circ Res, 2003. 92(2): p. 203-11.
152. Ecroyd, H. and J. A. Carver, The effect of small molecules in modulating the chaperone activity of alphaB-crystallin against ordered and disordered protein aggregation. FEBS J, 2008. 275(5): p. 935-47.
153. Derham, B. K., et al., Chaperone function of mutant versions of alpha A- and alpha B-crystallin prepared to pinpoint chaperone binding sites. Eur J Biochem, 2001. 268(3): p. 713-21.
154. Koteiche, H. A. and H. S. McHaourab, Mechanism of chaperone function in small heat-shock proteins. Phosphorylation-induced activation of two-mode binding in alphaB-crystallin. J Biol Chem, 2003. 278(12): p. 10361-7.
155. Kato, K., et al., Phosphorylation of alphaB-crystallin in mitotic cells and identification of enzymatic activities responsible for phosphorylation. J Biol Chem, 1998. 273(43): p. 28346-54.
156. Hartog, J. W. L., et al., Advanced glycation end-products (AGEs) and heart failure: Pathophysiology and clinical implications. European Journal of Heart Failure, 2007. 9(12): p. 1146-1155.
157. Jandeleit-Dahm, K. and M. E. Cooper, The role of AGEs in cardiovascular disease. Curr Pharm Des, 2008. 14(10): p. 979-86.
158. Mafia, K., et al., UV-A-induced structural and functional changes in human lens deamidated alphaB-crystallin. Mol V is, 2008. 14: p. 234-48.
159. Roquemore, E. P., et al., Dynamic O-GlcNAcylation of the small heat shock protein alpha B-crystallin. Biochemistry, 1996. 35(11): p. 3578-86.
160. Ray, P. S., et al., Transgene overexpression of alphaB crystallin confers simultaneous protection against cardiomyocyte apoptosis and necrosis during myocardial ischemia and reperfusion. Faseb J, 2001. 15(2): p. 393-402.
161. Morrison, L. E., et al., Roles for alphaB-crystallin and HSPB2 in protecting the myocardium from ischemia-reperfusion-induced damage in a KO mouse model. Am J Physiol Heart Circ Physiol, 2004. 286(3): p. H847-55.
162. Brady, J. P., et al., {alpha}B-Crystallin in Lens Development and Muscle Integrity: A Gene Knockout Approach. Invest. Ophthalmol. Vis. Sci., 2001. 42(12): p. 2924-2934.
163. Bova, M. P., et al., Mutation R120G in alphaB-crystallin, which is linked to a desmin-related myopathy, results in an irregular structure and defective chaperone-like function. Proc Natl Acad Sci USA, 1999. 96(11): p. 6137-42.
164. Sanbe, A., et al., Desmin-related cardiomyopathy in transgenic mice: a cardiac amyloidosis. Proc Natl Acad Sci USA, 2004. 101(27): p. 10132-6.
165. Perng, M. D., et al., The cardiomyopathy and lens cataract mutation in alphaB-crystallin alters its protein structure, chaperone activity, and interaction with intermediate filaments in vitro. J Biol Chem, 1999. 274(47): p. 33235-43.
166. Chiesi, M. and F. Bennardini, Determination of alpha B crystallin aggregation: a new alternative method to assess ischemic damage of the heart. Basic Res Cardiol, 1992. 87(1): p. 38-46.
167. Verschuure, P., et al., Translocation of small heat shock proteins to the actin cytoskeleton upon proteasomal inhibition. J Mol Cell Cardiol, 2002. 34(2): p. 117-28.
168. Golenhofen, N., et al., Binding of the stress protein alpha B-crystallin to cardiac myofibrils correlates with the degree of myocardial damage during ischemia/reperfusion in vivo. J Mol Cell Cardiol, 1999. 31(3): p. 569-80.
169. Bullard, B., et al., Association of the chaperone alphaB-crystallin with titin in heart muscle. J Biol Chem, 2004. 279(9): p. 7917-24.
170. Bennardini, F., A. Wrzosek, and M. Chiesi, Alpha B-crystallin in cardiac tissue. Association with actin and desmin filaments. Circ Res, 1992. 71(2): p. 288-94.
171. Golenhofen, N., et al., Ischemia-induced association of the stress protein alpha B-crystallin with I-band portion of cardiac titin. J Mol Cell Cardiol, 2002. 34(3): p. 309-19.
172. Perng, M., et al., Intermediate filament interactions can be altered by HSP27 and alphaB-crystallin. J Cell Sci, 1999. 112(13): p. 2099-2112.
173. Melkani, G. C., A. Cammarato, and S. I. Bernstein, alphaB-crystallin maintains skeletal muscle myosin enzymatic activity and prevents its aggregation under heat-shock stress. J Mol Biol, 2006. 358(3): p. 635-45.
174. Golenhofen, N., et al., Ischemia-induced increase of stiffness of alphaB-crystallin/HSPB2-deficient myocardium. Pflugers Arch, 2006. 451(4): p. 518-25.
175. LeWinter, M. M., et al., Cardiac titin: structure, functions and role in disease. Clin Chim Acta, 2007. 375(1-2): p. 1-9.
176. Balsam, L. B., T. Kofidis, and R. C. Robbins, Caspase-3 inhibition preserves myocardial geometry and long-term function after infarction. Journal of Surgical Research, 2005. 124(2): p. 194-200.
177. Mao, Y. W., et al., Human alphaA- and alphaB-crystallins bind to Bax and Bcl-X(S) to sequester their translocation during staurosporine-induced apoptosis. Cell Death Differ, 2004. 11(5): p. 512-26.
178. Kamradt, M. C., et al., The Small Heat Shock Protein {alpha}B-crystallin Is a Novel Inhibitor of TRAIL-induced Apoptosis That Suppresses the Activation of Caspase-3 10.1074/jbc.M413382200. J. Biol. Chem., 2005. 280(12): p. 11059-11066.
179. Kamradt, M. C., et al., The small heat shock protein alpha B-crystallin negatively regulates apoptosis during myogenic differentiation by inhibiting caspase-3 activation. J Biol Chem, 2002. 277(41): p. 38731-6.
180. Kamradt, M. C., F. Chen, and V. L. Cryns, The Small Heat Shock Protein alpha B-Crystallin Negatively Regulates Cytochrome c- and Caspase-8-dependent Activation of Caspase-3 by Inhibiting Its Autoproteolytic Maturation 10.1074/jbc.C100107200. J. Biol. Chem., 2001. 276(19): p. 16059-16063.
181. Fischer, P. and D. Hilfiker-Kleiner, Role of gp130-mediated signalling pathways in the heart and its impact on potential therapeutic aspects. Br J Pharmacol, 2008. 153 Suppl 1: p. S414-27.
182. Hausenloy, D. J., et al., Ischemic preconditioning protects by activating prosurvival kinases at reperfusion. Am J Physiol Heart Circ Physiol, 2005. 288(2): p. H971-6.
183. Kaiser, R. A., et al., Targeted inhibition of p38 mitogen-activated protein kinase antagonizes cardiac injury and cell death following ischemia-reperfusion in vivo. J Biol Chem, 2004. 279(15): p. 15524-30.
184. Zechner, D., et al., MKK6 activates myocardial cell NF-kappaB and inhibits apoptosis in a p38 mitogen-activated protein kinase-dependent manner. J Biol Chem, 1998. 273(14): p. 8232-9.
185. Ecroyd, H., et al., Mimicking phosphorylation of alphaB-crystallin affects its chaperone activity. Biochem J, 2007. 401(1): p. 129-41.
186. Golenhofen, N., et al., Ischemia-induced phosphorylation and translocation of stress protein alpha B-crystallin to Z lines of myocardium. Am J Physiol, 1998. 274(5 Pt 2): p. H1457-64.
187. Shoshan-Barmatz, V., N. Keinan, and H. Zaid, Uncovering the role of VDAC in the regulation of cell life and death. J Bioenerg Biomembr, 2008. 40(3): p. 183-91.
188. Lemasters, J. J. and E. Holmuhamedov, Voltage-dependent anion channel (VDAC) as mitochondrial governator—thinking outside the box. Biochim Biophys Acta, 2006. 1762(2): p. 181-90.
189. Granville, D. J. and R. A. Gottlieb, The mitochondrial voltage-dependent anion channel (VDAC) as a therapeutic target for initiating cell death. Curr Med Chem, 2003. 10(16): p. 1527-33.
190. Singh, B. N., et al., Association of alphaB-crystallin, a small heat shock protein, with actin: role in modulating actin filament dynamics in vivo. J Mol Biol, 2007. 366(3): p. 756-67.
191. Bowers, M. and H. Ardehali, TOM20 and the heartbreakers: evidence for the role of mitochondrial transport proteins in cardioprotection. J Mol Cell Cardiol, 2006. 41(3): p. 406-9.
192. Rane, M. J., et al., p38 Kinase-dependent MAPKAPK-2 activation functions as 3-phosphoinositide-dependent kinase-2 for Akt in human neutrophils. J Biol Chem, 2001. 276(5): p. 3517-23.
193. Baines, C. P., et al., Voltage-dependent anion channels are dispensable for mitochondrial-dependent cell death. Nat Cell Biol, 2007. 9(5): p. 550-5.
194. Benjamin, I. J., et al., CRYAB and HSPB2 deficiency alters cardiac metabolism and paradoxically confers protection against myocardial ischemia in aging mice. Am J Physiol Heart Circ Physiol, 2007. 293(5): p. H3201-9.
195. White, H. D. and D. P. Chew, Acute myocardial infarction. The Lancet. 372(9638): p. 570-584.
196. Preville, X., et al., Mammalian small stress proteins protect against oxidative stress through their ability to increase glucose-6-phosphate dehydrogenase activity and by maintaining optimal cellular detoxifying machinery. Exp Cell Res, 1999. 247(1): p. 61-78.
197. Mehlen, P., et al., Human hsp27, Drosophila hsp27 and human alphaB-crystallin expression-mediated increase in glutathione is essential for the protective activity of these proteins against TNFalpha-induced cell death. EMBO J, 1996. 15(11): p. 2695-706.
198. Raab, W. and B. Gmeiner, The inhibition of glucose-6-phosphate dehydrogenase activity by dithranol (anthralin), zinc ions and/or salicylic acid. Arch Dermatol Forsch, 1974. 251(2): p. 87-94.
199. Baek, S. H., et al., Role of small heat shock protein HSP25 in radioresistance and glutathione-redox cycle. J Cell Physiol, 2000. 183(1): p. 100-7.
200. Kanai, A., et al., Differing roles of mitochondrial nitric oxide synthase in cardiomyocytes and urothelial cells. Am J Physiol Heart Circ Physiol, 2004. 286(1): p. H13-21.
201. Zhao, M. and R. Weissleder, Intracellular cargo delivery using tat peptide and derivatives. Med Res Rev, 2004. 24(1): p. 1-12.
202. Foerg, C. and H. P. Merkle, On the biomedical promise of cell penetrating peptides: limits versus prospects. J Pharm Sci, 2008. 97(1): p. 144-62.
203. Patel, L. N., J. L. Zaro, and W. C. Shen, Cell penetrating peptides: intracellular pathways and pharmaceutical perspectives. Pharm Res, 2007. 24(11): p. 1977-92.
204. Ho, A., et al., Synthetic protein transduction domains: enhanced transduction potential in vitro and in vivo. Cancer Res, 2001. 61(2): p. 474-7.
205. Kwon, J. H., et al., Protective effect of heat shock protein 27 using protein transduction domain-mediated delivery on ischemia/reperfusion heart injury. Biochemical and Biophysical Research Communications, 2007. 363(2): p. 399-404.
206. Gustafsson, A. B., et al., TAT Protein Transduction Into Isolated Perfused Hearts: TAT-Apoptosis Repressor With Caspase Recruitment Domain Is Cardioprotective 10.1161/01.CIR.0000023943.50821.F7. Circulation, 2002. 106(6): p. 735-739.
207. Schwarze, S. R., et al., In vivo protein transduction: delivery of a biologically active protein into the mouse. Science, 1999. 285(5433): p. 1569-72.
208. Horwitz, J., Alpha-crystallin can function as a molecular chaperone. Proc Natl Acad Sci USA, 1992. 89(21): p. 10449-53.
209. Hess, J. F. and P. G. FitzGerald, Protection of a restriction enzyme from heat inactivation by [alpha]-crystallin. Mol V is, 1998. 4: p. 29.
210. Marini, I., et al., Complete protection by alpha-crystallin of lens sorbitol dehydrogenase undergoing thermal stress. J Biol Chem, 2000. 275(42): p. 32559-65.
211. Go, Y. M. and D. P. Jones, Redox compartmentalization in eukaryotic cells. Biochim Biophys Acta, 2008. 1780(11): p. 1273-90.
212. Pinz, I., et al., Unmasking different mechanical and energetic roles for the small heat shock proteins CryAB and HSPB2 using genetically modified mouse hearts. Faseb J, 2008. 22(1): p. 84-92.

While the invention is susceptible to various modifications and alternative forms, specific examples thereof have been shown by way of example in the drawings and are herein described in detail. It should be understood, however, that the invention is not to be limited to the particular forms or methods disclosed, but to the contrary, the invention is to cover all modifications, equivalents, and alternatives falling within the spirit and scope of the appended claims.

1. An isolated, synthetic or recombinant alpha B-Crystallin (αBC) polypeptide comprising or consisting of:
(a) (i) an alpha B-Crystallin (αBC) amino acid sequence in which the serine at position 59, or equivalent, has been changed to glutamate to mimic phosphorylation, and the serine at positions 19 and 45 or equivalents, or the serine at position 19, or the serine at position 45, has/have been changed to glycine or to an amino acid that cannot be phosphorylated in vivo to prevent in vivo phosphorylation of those residues; or (ii)) an alpha B-Crystallin (αBC) amino acid sequence in which the serine at position 59, or equivalent, has been changed to glutamate to mimic phosphorylation, and the serine at positions 19 and 45, or the serine at position 19, or the serine at position 45, has/have been changed to alanine to prevent phosphorylation of those residues; or (iii) an alpha B-Crystallin (αBC) amino acid sequence wherein the serine at positions 19 and 45, or the serine at position 19, or the serine at position 45, has/have been changed to alanine or glycine to prevent phosphorylation of those residues; or
(b) a polypeptide comprising or consisting of
(SEQ ID NO: 1)
D IAIHHPWIRR PFFPFHAPSR LFDQFFGEHL LESDLFSTAT
SLAPFYLRPP SFLRAPEWID TGLSEMRMEK DRFSVNLDVK
HFSPEELKVK VLGDVIEVHG KHEERQDEHG FISREFHRKY
RIPADVDPLT ITSSLSSDGV LTVNGPRKQA SGPERTIPIT
REEKPAVTAA PKK;
or
(SEQ ID NO: 6)
D IAIHHPWIRR PFFPFHGPSR LFDQFFGEHL LESDLFSTAT
SLGPFYLRPP SFLRAPEWID TGLSEMRMEK DRFSVNLDVK
HFSPEELKVK VLGDVIEVHG KHEERQDEHG FISREFHRKY
RIPADVDPLT ITSSLSSDGV LTVNGPRKQA SGPERTIPIT
REEKPAVTAA PKK;
or
(SEQ ID NO: 7)
D IAIHHPWIRR PFFPFHAPSR LFDQFFGEHL LESDLFSTAT
SLGPFYLRPP SFLRAPEWID TGLSEMRMEK DRFSVNLDVK
HFSPEELKVK VLGDVIEVHG KHEERQDEHG FISREFHRKY
RIPADVDPLT ITSSLSSDGV LTVNGPRKQA SGPERTIPIT
REEKPAVTAA PKK;
or
(SEQ ID NO: 8)
D IAIHHPWIRR PFFPFHGPSR LFDQFFGEHL LESDLFSTAT
SLAPFYLRPP SFLRAPEWID TGLSEMRMEK DRFSVNLDVK
HFSPEELKVK VLGDVIEVHG KHEERQDEHG FISREFHRKY
RIPADVDPLT ITSSLSSDGV LTVNGPRKQA SGPERTIPIT
REEKPAVTAA PKK.

2. The alpha B-Crystallin (αBC) polypeptide of claim 1, further comprising (a) at least one protein transduction peptide domain to facilitate the delivery of the αBC protein across a cell membrane; (b) a peptide comprising a plurality of arginines; (c) a peptide comprising 5, 6, 7, 8, 9, or 10 or more arginines; or (d) a peptide consisting of 5, 6, 7, 8, 9, or 10 or more arginines.

3. The alpha B-Crystallin (αBC) polypeptide of claim 1, wherein the αBC polypeptide is reversibly coupled to the peptide.

4. The alpha B-Crystallin (αBC) polypeptide of claim 1, further comprising at least one a cysteine residue.

5. The alpha B-Crystallin (αBC) polypeptide of claim 4, wherein the alpha B-Crystallin (αBC) polypeptide is coupled to the peptide via a disulfide bond between cysteine residues.

6. A composition comprising the alpha B-Crystallin (αBC) polypeptide of claim 1.

7. The composition of claim 6, formulated as a liquid, gel or powder.

8. A pharmaceutical composition comprising the alpha B-Crystallin (αBC) polypeptide of claim 1.

9. The alpha B-Crystallin (αBC) polypeptide of claim 1 formulated as a pharmaceutical composition to protect cells from ischemia/reperfusion injury.

10. The alpha B-Crystallin (αBC) polypeptide of claim 9, wherein the pharmaceutical composition is formulated to protect heart or muscle cells from ischemia/reperfusion (FR) injury.

11. A method for ameliorating a cell from ischemia/reperfusion injury or protecting a cell from ischemia/reperfusion injury, comprising:
(a) providing the alpha B-Crystallin (αBC) polypeptide of claim 1; and
(b) administering (contacting) the alpha B-Crystallin (αBC) polypeptide of (a) to a cell, tissue, organ or individual in need thereof.

12. The method of claim 11, wherein the cell, tissue, organ is a heart, heart tissue or heart muscle or cardiac muscle.

13. An isolated, recombinant or synthetic nucleic acid encoding the alpha B-Crystallin (αBC) polypeptide of claim 1.

14. An expression vehicle or vector comprising the nucleic acid of claim 13.

15. A host cell comprising: the isolated, recombinant or synthetic nucleic acid of claim 13.

16. A non-human transgenic animal comprising the isolated, recombinant or synthetic nucleic acid of claim 13.

17. A host cell comprising a recombinant or synthetic alpha B-Crystallin (αBC) polypeptide of claim 1.

18. A host cell comprising an expression vehicle or vector of claim 14.

19. A method for ameliorating a cell from ischemia/reperfusion injury or protecting a cell from ischemia/reperfusion injury, comprising:
(a) providing the pharmaceutical composition of claim 8; and
(b) administering (contacting) the pharmaceutical composition to a cell, tissue, organ or individual in need thereof.

20. An isolated, synthetic or recombinant polypeptide comprising or consisting of:
(SEQ ID NO: 1)
D IAIHHPWIRR PFFPFHAPSR LFDQFFGEHL LESDLFSTAT
SLAPFYLRPP SFLRAPEWID TGLSEMRMEK DRFSVNLDVK
HFSPEELKVK VLGDVIEVHG KHEERQDEHG FISREFHRKY
RIPADVDPLT ITSSLSSDGV LTVNGPRKQA SGPERTIPIT
REEKPAVTAA PKK;
or
(SEQ ID NO: 6)
D IAIHHPWIRR PFFPFHGPSR LFDQFFGEHL LESDLFSTAT
SLGPFYLRPP SFLRAPEWID TGLSEMRMEK DRFSVNLDVK
HFSPEELKVK VLGDVIEVHG KHEERQDEHG FISREFHRKY
RIPADVDPLT ITSSLSSDGV LTVNGPRKQA SGPERTIPIT
REEKPAVTAA PKK;
or
(SEQ ID NO: 7)
D IAIHHPWIRR PFFPFHAPSR LFDQFFGEHL LESDLFSTAT
SLGPFYLRPP SFLRAPEWID TGLSEMRMEK DRFSVNLDVK
HFSPEELKVK VLGDVIEVHG KHEERQDEHG FISREFHRKY
RIPADVDPLT ITSSLSSDGV LTVNGPRKQA SGPERTIPIT
REEKPAVTAA PKK;
or
(SEQ ID NO: 8)
D IAIHHPWIRR PFFPFHGPSR LFDQFFGEHL LESDLFSTAT
SLAPFYLRPP SFLRAPEWID TGLSEMRMEK DRFSVNLDVK
HFSPEELKVK VLGDVIEVHG KHEERQDEHG FISREFHRKY
RIPADVDPLT ITSSLSSDGV LTVNGPRKQA SGPERTIPIT
REEKPAVTAA PKK.


SEQUENCE LISTING (8 sequences)

SEQ ID NO: 1 - protein, 174 aa, artificial sequence
  synthetic polypeptide
DIAIHHPWIRRPFFPFHAPSRLFDQFFGEHLLESDLFSTATSLAPFYLRPPSFLRAPEWIDTGLSEMRMEKDRFSVNLDVKHFSPEELKVKVLGDVIEVHGKHEERQDEHGFISREFHRKYRIPADVDPLTITSSLSSDGVLTVNGPRKQASGPERTIPITREEKPAVTAAPKK

SEQ ID NO: 2 - protein, 9 aa, artificial sequence
  synthetic peptide
KDRCGSELE

SEQ ID NO: 3 - protein, 30 aa, artificial sequence
  synthetic peptide
MRGSHHHHHHGMASMTGGQQMGRDLYDDDD

SEQ ID NO: 4 - protein, 10 aa, artificial sequence
  synthetic peptide
RRRRRRRRRC

SEQ ID NO: 5 - protein, 8 aa, artificial sequence
  synthetic peptide
DRCGSELE

SEQ ID NO: 6 - protein, 174 aa, artificial sequence
  synthetic polypeptide
DIAIHHPWIRRPFFPFHGPSRLFDQFFGEHLLESDLFSTATSLGPFYLRPPSFLRAPEWIDTGLSEMRMEKDRFSVNLDVKHFSPEELKVKVLGDVIEVHGKHEERQDEHGFISREFHRKYRIPADVDPLTITSSLSSDGVLTVNGPRKQASGPERTIPITREEKPAVTAAPKK

SEQ ID NO: 7 - protein, 174 aa, artificial sequence
  synthetic polypeptide
DIAIHHPWIRRPFFPFHAPSRLFDQFFGEHLLESDLFSTATSLGPFYLRPPSFLRAPEWIDTGLSEMRMEKDRFSVNLDVKHFSPEELKVKVLGDVIEVHGKHEERQDEHGFISREFHRKYRIPADVDPLTITSSLSSDGVLTVNGPRKQASGPERTIPITREEKPAVTAAPKK

SEQ ID NO: 8 - protein, 174 aa, artificial sequence
  synthetic polypeptide
DIAIHHPWIRRPFFPFHGPSRLFDQFFGEHLLESDLFSTATSLAPFYLRPPSFLRAPEWIDTGLSEMRMEKDRFSVNLDVKHFSPEELKVKVLGDVIEVHGKHEERQDEHGFISREFHRKYRIPADVDPLTITSSLSSDGVLTVNGPRKQASGPERTIPITREEKPAVTAAPKK